(19) United States
(12) Patent Application Publication
(10) Pub. No.: US 2013/0142792 A1
(43) Pub. Date: Jun. 6, 2013

(54) RAGE Fusion Protein Compositions And Methods Of Use

(71) Applicants: Jeffrey C. Webster, Jamestown, NC (US); Gregory T. Bleck, Cross Plains, WI (US); Madan Katragadda, Bedford, MA (US); Manoj Rajadhyaksha, Colchester, CT (US); Bernard N. Violand, Wildwood, MO (US); Jo-Ann Wentland, Pawcatuck, CT (US); Linda Marie Rothlein, Greensboro, NC (US)

(72) Inventors: Robert Rothlein, Greensboro, NC (US); Jeffrey C. Webster, Jamestown, NC (US); Gregory T. Bleck, Cross Plains, WI (US); Madan Katragadda, Bedford, MA (US); Manoj Rajadhyaksha, Colchester, CT (US); Bernard N. Violand, Wildwood, MO (US); Jo-Ann Wentland, Pawcatuck, CT (US)

(21) Appl. No.: 13/062,395

(22) PCT Filed: Oct. 19, 2010
(86) PCT No.: PCT/US10/53157

§ 371 Date: Oct. 3, 2012

Related U.S. Application Data
(60) Provisional application No. 61/305,706, filed on Feb. 18, 2010.

(51) Int. Cl. C07K 14/705; C07K 16/00

(52) U.S. Cl. CPC C07K 14/70503; C07K 16/00
USPC 424/134.1; 530/387.3; 536/23.4; 435/320.1; 435/325; 435/361; 435/369; 435/354; 435/367; 435/352; 435/365; 435/370; 435/371

(57) ABSTRACT

Disclosed are fusion proteins comprising a RAGE polypeptide, wherein the RAGE polypeptide comprises a fragment of a mammalian wild type RAGE peptide and at least one point mutation in the RAGE polypeptide portion of the fusion protein relative to the wild type RAGE peptide. The point mutation may remove and/or alter a glycosylation site or an enzyme cleavage site. Also disclosed are nucleic acids encoding such proteins as well as methods of using such proteins for treating RAGE-mediated pathologies.

### CROSS REFERENCE TO RELATED APPLICATION

This application claims priority under 35 U.S.C. §119(e) to U.S. Provisional Patent Application No. 61/305,706, filed on Feb. 18, 2010 and claims priority under 35 U.S.C. 365(c) to International Application No. PCT/US2010/032270, filed Apr. 23, 2010. International Application No. PCT/US2010/032270, filed Apr. 23, 2010 claims priority under 35 U.S.C. §119(e) to U.S. Provisional Patent Application No. 61/305,706, filed on Feb. 18, 2010. The disclosures of U.S. Provisional Patent Application No. 61/305,706, and International Application No. PCT/US2010/032270 are incorporated by reference in their entireties herein.

### FIELD OF THE INVENTION

The present invention relates to fusion proteins comprising the receptor for advanced glycation end products (“RAGE”), nucleic acids encoding RAGE fusion proteins, and methods of using such proteins.

### BACKGROUND

Incubation of proteins or lipids with aldose sugars results in nonenzymatic glycation and oxidation of amino groups on proteins to form Amadori adducts. Over time, the adducts undergo additional rearrangements, dehydrations, and cross-linking with other proteins to form complexes known as advanced glycation end products (“AGEs”). Factors which promote formation of AGEs include delayed protein turnover (e.g. as in amyloidoses), accumulation of macromolecules having high lysine content, and high blood glucose levels (e.g. as in diabetes) (Hori et al., J. Biol. Chem. 270: 25752-761, (1995)). AGEs have been implicated in a variety of disorders including complications associated with diabetes and normal aging.

AGEs display specific and saturable binding to cell surface receptors on monocytes, macrophages, endothelial cells of the microvasculature, smooth muscle cells, mesengial cells, and neurons. The receptor for advanced glycation end products (“RAGE”) is a member of the immunoglobulin supergene family of molecules. The extracellular (N-terminal) domain of RAGE includes three immunoglobulin-type regions: one V (variable) type domain followed by two C-type (constant) domains (Neeper et al., J. Biol. Chem., 267:14998-15004 (1992); Schmidt et al., Circ. (Suppl.) 96#194 (1997)). A single transmembrane spanning domain and a short, highly charged cytosolic tail follow the extracellular domain. The N-terminal, extracellular domain can be isolated by proteolysis of RAGE or by molecular biological approaches to generate soluble RAGE (sRAGE) comprised of the V and C domains.

RAGE is expressed on multiple cell types including leukocytes, neurons, microglial cells and vascular endothelium (e.g., Hori et al., J. Biol. Chem., 270:25752-761 (1995)). Increased levels of RAGE are also found in aging tissues (Schleicher et al., J. Clin. Invest., 99 (3): 457-468 (1997)), and the diabetic retina, vasculature and kidney (Schmidt et al., Nature Med., 1:1002-1004 (1995)).

In addition to AGEs, other compounds can bind to and modulate RAGE. RAGE binds to multiple functionally and structurally diverse ligands including amyloid beta (Aβ), serum amyloid A (SAA), Advanced Glycation End products (AGEs), S100 (a proinflammatory member of the Calgranulin family), carboxymethyl lysine (CML), amphoterin and CD11b/CD18 (Bucciarelli et al., Cell Mol. Life Sci., 59:1117-128 (2002); Chavakis et al., Microbes Infect., 6:1219-1225 (2004); Kokkola et al., Scand. J. Immunol., 61:1-9 (2005); Schmidt et al., J. Clin. Invest., 108:949-955 (2001); Rocken et al., Am. J. Pathol., 162:1213-1220 (2003)).

Binding of ligands such as AGEs, S100/calgranulin, β-amyloid, CML (N^{ε}-Carboxymethyl lysine), and amphoterin to RAGE has been shown to modify expression of a variety of genes. These interactions may then initiate signal transduction mechanisms including p38 activation, p21ras, MAP kinases, Erk1-2 phosphorylation, and the activation of the transcriptional mediator of inflammatory signaling, NF-κB (Yeh et al., Diabetes, 50:1495-1504 (2001)). For example, in many cell types, interaction between RAGE and its ligands can generate oxidative stress, which thereby results in activation of the free radical sensitive transcription factor NF-κB, and the activation of NF-κB regulated genes, such as the cytokines IL-1β and TNF-α. Furthermore, RAGE expression is upregulated via NF-κB and shows increased expression at sites of inflammation or oxidative stress (Tanaka et al., J. Biol. Chem., 275:25781-25790 (2000)). Thus, an ascending and often detrimental spiral may be fueled by a positive feedback loop initiated by ligand binding.

Activation of RAGE in different tissues and organs can lead to a number of pathophysiological consequences. RAGE has been implicated in a variety of conditions including: acute and chronic inflammation (Hofmann et al., Cell 97:889-901 (1999)), the development of diabetic late complications such as increased vascular permeability (Wautier et al., J. Clin. Invest., 97:238-243 (1995)), nephropathy (Teillet et al., J. Am. Soc. Nephrol., 11:1488-1497 (2000)), arteriosclerosis (Vlassara et. al., The Finnish Medical Society DUODECIM, Ann. Med., 28:419-426 (1996)), and retinopathy (Hammes et al., Diabetologia, 42:603-607 (1999)). RAGE has also been implicated in Alzheimer's disease (Yan et al., Nature, 382:685-691 (1996)), and in tumor invasion and metastasis (Taguchi et al., Nature, 405:354-357 (2000)).

Despite the broad expression of RAGE and its apparent pleiotropic role in multiple diverse disease models, RAGE does not appear to be essential to normal development. For example, RAGE knockout mice are without an overt abnormal phenotype, suggesting that while RAGE can play a role in disease pathology when stimulated chronically, inhibition of RAGE does not appear to contribute to any unwanted acute phenotype (Liliensiek et al., J. Clin. Invest. 113:1641-50 (2004)).

Antagonizing binding of physiological ligands to RAGE may down-regulate the pathophysiological changes brought about by excessive concentrations of AGEs and other RAGE ligands. By reducing binding of endogenous ligands to RAGE, symptoms associated with RAGE-mediated disorders may be reduced. Soluble RAGE (sRAGE) is able to effectively antagonize the binding of RAGE ligands to RAGE. However, sRAGE can have a half-life when administered in vivo that may be too short to be therapeutically useful for one or more disorders.

Fusion proteins comprising a RAGE ligand domain linked to a second polypeptide may provide the ability to antagonize natural RAGE ligands, while having a half-life that is long enough to be therapeutically useful. Still, in certain embodiments, such fusion proteins may be modified by protein processing (e.g., glycosylation and/or enzyme mediated cleavage) during expression of the protein in large scale bioreactors. Such processing may reduce the half-life of the fusion protein as compared to molecules that are not modified in any manner. Thus, the compounds and methods of the present invention address the need to develop compounds that antagonize the binding of AGEs and other physiological ligands to the RAGE receptor where the compound has a desirable pharmacokinetic profile.

### SUMMARY

Embodiments of the present invention relate to fusion proteins comprising the receptor for advanced glycation end products (“RAGE”). In certain embodiments, the RAGE fusion proteins comprise RAGE proteins having at least one point mutation relative to wild type RAGE, and nucleic acid sequences encoding same. Yet other embodiments of the invention comprise optimized nucleic acid sequences and contructs for making the fusion proteins of the present invention, as well as compositions and methods of use of such RAGE fusion proteins for the treatment of disorders.

In certain embodiments, the present invention comprises a fusion protein comprising a Receptor for Advanced Glycation Endproducts (RAGE) polypeptide linked to an immunoglobulin polypeptide, wherein the RAGE polypeptide comprises a fragment of a mammalian RAGE having a ligand binding domain, and wherein the fusion protein has at least one mutation relative to the wild-type sequence in at least one of the RAGE polypeptide or the immunoglobulin polypeptide, wherein the mutation removes and/or alters at least one of a glycosylation site or an enzyme cleavage site. In some embodiments, the enzyme cleavage site is a furin cleavage site. In certain embodiments, the immunoglobulin polypeptide comprises a C_{H}2 domain or a fragment of a C_{H}2 domain. Also, in certain embodiments, at least a portion of the hinge region is not included in the C_{H}2 domain fragment. Also, in certain embodiments, the C_{H}2 domain is linked via its C-terminus to the N-terminus of a C_{H}3 domain of an immunoglobulin polypeptide.

For example, in some embodiments, the present invention provides a fusion protein comprising a RAGE polypeptide and an immunoglobulin polypeptide, wherein: a) the RAGE polypeptide comprises a fragment of a mammalian wild type RAGE peptide comprising a RAGE ligand binding domain, and wherein the RAGE polypeptide comprises a point mutation or mutations at one or more of residues relative to the wild type RAGE peptide to remove and/or alter at least one glycosylation site; and b) the immunoglobulin polypeptide comprises at least a fragment of a C_{H}2 domain. In an embodiment, at least a portion of the hinge region not included as part of the fragment of a C_{H}2 domain. In an embodiment, the glycosylation site that is removed and/or altered by the mutation is in the ligand binding domain of the RAGE polypeptide.

In other embodiments, the present invention provides a fusion protein comprising a RAGE polypeptide and an immunoglobulin polypeptide, wherein: a) the RAGE polypeptide comprises a fragment of a mammalian wild type RAGE peptide comprising a RAGE ligand binding domain, and wherein the RAGE polypeptide comprises a point mutation or mutations at one or more of residues relative to the wild type RAGE peptide to remove and/or alter an enzyme cleavage site; and b) the immunoglobulin polypeptide comprises at least a fragment of a C_{H}2 domain and a C_{H}3 domain of an immunoglobulin. In an embodiment, the fragment of a C_{H}2 domain does not include at least a portion of the hinge region. In certain embodiments, the enzyme cleavage site is a furin cleavage site.

In other embodiments, the present invention provides a fusion protein comprising a RAGE polypeptide and an immunoglobulin polypeptide, wherein: a) the RAGE polypeptide comprises a fragment of a mammalian wild type RAGE peptide comprising a RAGE ligand binding domain, and wherein the RAGE polypeptide comprises: i) a point mutation or mutations at one or more of residues relative to the wild type RAGE peptide to remove and/or alter at least one glycosylation site, and ii) a point mutation or mutations at one or more of residues relative to the wild type RAGE peptide to remove and/or alter a furin cleavage site; and b) the immunoglobulin polypeptide comprises at least a fragment of a C_{H}2 domain, and a C_{H}3 domain of an immunoglobulin. In an embodiment, the fragment of a C_{H}2 domain does not include at least a portion of the hinge region.

In another embodiment, the present invention comprises nucleic acids encoding the RAGE fusion proteins as described herein. Expression vectors comprising these nucleic acids, as well as host cells transfected with such vectors are also provided.

In other embodiments, the present invention comprises methods and compositions for treating a RAGE-mediated disorder in a subject. The method may comprise administering a RAGE fusion protein of the present invention to the subject. The composition may comprise a RAGE fusion protein of the present invention and a pharmaceutically acceptable carrier.

Various advantages may be associated with particular embodiments of the fusion protein of the present invention. In one embodiment, the RAGE fusion proteins of the present invention may have increased metabolic stability relative to wild type RAGE fusion protein (i.e., a RAGE fusion protein without one or more point mutations to remove and/or alter either a glycosylation site or an enzyme cleavage site). Also, the RAGE fusion proteins of the present invention may exhibit high-affinity (i.e., physiologically relevant) binding for RAGE ligands. In certain embodiments, the RAGE fusion proteins of the present invention may bind to RAGE ligands (e.g., S100b, amyloid-beta, carboxymethyl-lysine) with affinities in the high nanomolar to low micromolar range and with greater affinity than a similar wild type RAGE fusion protein. By binding with high affinity to physiological RAGE ligands, the RAGE fusion proteins of the present invention may be used to inhibit binding of endogenous ligands to RAGE, thereby providing a means to ameliorate RAGE-mediated diseases.

There are additional features of the invention which will be described hereinafter. It is to be understood that the invention is not limited in its application to the details set forth in the following claims, description and figures. The invention is capable of other embodiments and of being practiced or carried out in various ways.

### BRIEF DESCRIPTION OF THE FIGURES

Various features, aspects and advantages of the present invention will become more apparent with reference to the following figures.

FIG. 1 shows various RAGE sequences and immunoglobulin sequences in accordance with alternate embodiments of the present invention: Panel A, SEQ ID NO: 1, the amino acid sequence for human RAGE; and SEQ ID NO: 2, the amino acid sequence for human RAGE without the signal sequence of amino acids 1-22; Panel B, SEQ ID NO: 3, the amino acid sequence for human RAGE without the signal sequence of amino acids 1-23; Panel C, SEQ ID NO: 4, the amino acid sequence of human sRAGE; SEQ ID NO: 5, the amino acid sequence of human sRAGE without the signal sequence of amino acids 1-22, and SEQ ID NO: 6, the amino acid sequence of human sRAGE without the signal sequence of amino acids 1-23; Panel D, SEQ ID NO: 7, an amino acid sequence comprising the V-domain of human RAGE; SEQ ID NO: 8, an alternate amino acid sequence comprising the V-domain of human RAGE; SEQ ID NO: 9, an N-terminal fragment of the V-domain of human RAGE; SEQ ID NO: 10, an alternate N-terminal fragment of the V-domain of human RAGE; SEQ ID NO: 11, the amino acid sequence for amino acids 124-221 of human RAGE; SEQ ID NO: 12, the amino acid sequence for amino acids 227-317 of human RAGE; SEQ ID NO: 13, the amino acid sequence for amino acids 23-123 of human RAGE; Panel E, SEQ ID NO: 14, the amino acid sequence for amino acids 24-123 of human RAGE; SEQ ID NO: 15, the amino acid sequence for amino acids 23-136 of human RAGE; SEQ ID NO: 16, the amino acid sequence for amino acids 24-136 of human RAGE; SEQ ID NO: 17, the amino acid sequence for amino acids 23-226 of human RAGE; SEQ ID NO: 18, the amino acid sequence for amino acids 24-226 of human RAGE; Panel F, SEQ ID NO: 19, the amino acid sequence for amino acids 23-251 of human RAGE; SEQ ID NO: 20, the amino acid sequence for amino acids 24-251 of human RAGE; SEQ ID NO: 21, a RAGE interdomain linker; SEQ ID NO: 22, a second RAGE interdomain linker; SEQ ID NO: 23, a third RAGE interdomain linker; SEQ ID NO: 24, a fourth RAGE interdomain linker; Panel G, SEQ ID NO: 25, DNA encoding human RAGE amino acids 1-118; SEQ ID NO: 26, DNA encoding human RAGE amino acids 1-123; and SEQ ID NO: 27, DNA encoding human RAGE amino acids 1-136; Panel H, SEQ ID NO: 28, DNA encoding human RAGE amino acids 1-230; and SEQ ID NO: 29, DNA encoding human RAGE amino acids 1-251; Panel I, SEQ ID NO: 38, a partial amino acid sequence for the C_{H}2 and C_{H}3 domains of human IgG; SEQ ID NO:39, DNA encoding a portion of the human C_{H}2 and C_{H}3 domains of human IgG; SEQ ID NO: 40, an amino acid sequence for the C_{H}2 and C_{H}3 domains of human IgG; Panel J, SEQ ID NO: 41, a DNA encoding the human C_{H}2 and C_{H}3 domains of human IgG; SEQ ID NO: 42, an amino acid sequence for the C_{H}2 domain of human IgG; SEQ ID NO: 43, an amino acid sequence for the C_{H}3 domain of human IgG; and SEQ ID NO: 44, a fifth RAGE interdomain linker; Panel K, SEQ ID NO: 45, the amino acid sequence of human sRAGE without the signal sequence of amino acids 1-23 where the glutamine residue at the N-terminus has cyclized to form pyroglutamic acid, SEQ ID NO: 46, an alternate amino acid sequence comprising the V-domain of human sRAGE where the glutamine residue at the N-terminus has cyclized to form pyroglutamic acid, SEQ ID NO: 47, an alternate N-terminal fragment of the V-domain of human RAGE where the glutamine residue at the N-terminus has cyclized to form pyroglutamic acid, SEQ ID NO: 48, the amino acid sequence for amino acids 24-123 of human RAGE where the glutamine residue at the N-terminus has cyclized to form pyroglutamic acid; Panel L, SEQ ID NO: 49, the amino acid sequence for amino acids 24-136 of human RAGE where the glutamine residue at the N-terminus has cyclized to form pyroglutamic acid, SEQ ID NO: 50, the amino acid sequence for amino acids 24-226 of human RAGE where the glutamine residue at the N-terminus has cyclized to form pyroglutamic acid, SEQ ID NO: 51, the amino acid sequence for amino acids 24-251 of human RAGE where the glutamine residue at the N-terminus has cyclized to form pyroglutamic acid; Panel M, SEQ ID NO: 52, an alternate DNA sequence encoding a portion of the human C_{H}2 and C_{H}3 domains of human IgG in SEQ ID NO: 38, and SEQ ID NO: 53, an alternate DNA sequence encoding the human C_{H}2 and C_{H}3 domains of human IgG in SEQ ID NO: 40.

FIG. 2 shows alternate DNA sequences SEQ ID NO: 30 (Panel A) and SEQ ID NO: 54 (Panel B) that encode a first RAGE fusion protein (TTP-4000) coding region in accordance with an embodiment of the present invention. Coding sequence 1-753 highlighted in bold encodes RAGE N-terminal protein sequence whereas sequence 754-1386 encodes human IgG Fc (γ1) protein sequence without the hinge region.

FIG. 3 shows alternate DNA sequences SEQ ID NO: 31 (Panel A) and SEQ ID NO: 55 (Panel B) that encode a second RAGE fusion protein (TTP-3000) coding region in accordance with an embodiment of the present invention. Coding sequence 1-408 highlighted in bold encodes RAGE N-terminal protein sequence, whereas sequence 409-1041 codes human IgG Fc (γ1) protein sequence without the hinge region.

FIG. 4 shows the amino acid sequences, SEQ ID NO: 32, SEQ ID NO: 33, SEQ ID NO: 34, and SEQ ID NO: 56 that each encode a four domain RAGE fusion protein in accordance with alternate embodiments of the present invention. RAGE sequence is highlighted with bold font.

FIG. 5 shows the amino acid sequences, SEQ ID NO: 35, SEQ ID NO: 36, SEQ ID NO: 37, and SEQ ID NO: 57 that each encode a three domain RAGE fusion protein in accordance with alternate embodiments of the present invention. RAGE sequence is highlighted with bold font.

FIG. 6 shows various RAGE fusion proteins comprising RAGE polypeptide sequences and immunoglobulin polypeptide sequences in accordance with alternate embodiments of the present invention. The RAGE fusion proteins correspond to the amino acid sequence of the RAGE fusion protein of SEQ ID NO: 34 (TTP4000), wherein the signal sequence of amino acids 1-23 has been removed and the RAGE fusion protein comprises a point mutation or mutations to remove a glycosylation site and/or a furin cleavage site based on number that begins with amino acid 24 relative to the full-length wild-type RAGE sequence (SEQ ID NO: 1). The sequences are provided as follows: Panel A, SEQ ID NO: 62, the amino acid sequence for the TTP4000 RAGE fusion protein with an N2Q (i.e., corresponding to N25Q in the full-length wild-type sequence) point mutation; SEQ ID NO: 63, the amino acid sequence for the TTP4000 RAGE fusion protein with an N58Q point mutation (i.e., corresponding to N81Q in the full-length wild-type sequence); SEQ ID NO: 64, the amino acid sequence for the TTP4000 RAGE fusion protein with N2Q and N58Q point mutations; SEQ ID NO: 65, the amino acid sequence for the TTP4000 RAGE fusion protein with an R195A point mutation; Panel B, SEQ ID NO: 66, the amino acid sequence for the TTP4000 RAGE fusion protein with an R195K point mutation; SEQ ID NO: 67, the amino acid sequence for the TTP4000 RAGE fusion protein with an R198A point mutation; SEQ ID NO: 68, the amino acid sequence for the TTP4000 RAGE fusion protein with an R198K point mutation; SEQ ID NO: 69, the amino acid sequence for the TTP4000 RAGE fusion protein with an R198H point mutation; Panel C, SEQ ID NO: 70, the amino acid sequence for the TTP4000 RAGE fusion protein with an R198T point mutation; SEQ ID NO: 71, the amino acid sequence for the TTP4000 RAGE fusion protein with an N2Q and an R198A point mutation; SEQ ID NO: 72, the amino acid sequence for the TTP4000 RAGE fusion protein with N58Q and R198A point mutations; SEQ ID NO: 73, the amino acid sequence for the TTP4000 RAGE fusion protein with N2Q, N58Q, and R198A point mutations; Panel D, SEQ ID NO: 74, the amino acid sequence for the TTP4000 RAGE fusion protein with an N288Q point mutation; SEQ ID NO: 75, the amino acid sequence for the TTP4000 RAGE fusion protein with N2Q and N288Q point mutations, SEQ ID NO: 76, the amino acid sequence for the TTP4000 RAGE fusion protein with N58Q and N288Q point mutations; SEQ ID NO: 77, the amino acid sequence for the TTP4000 RAGE fusion protein with N2Q, N58Q, and N288Q point mutations; Panel E, SEQ ID NO: 78, the amino acid sequence for the TTP4000 RAGE fusion protein with N2Q, R198A, and N288Q point mutations; SEQ ID NO: 79, the amino acid sequence for the TTP4000 RAGE fusion protein with N58Q, R198A, and N288Q point mutations; SEQ ID NO: 80, the amino acid sequence for the TTP4000 RAGE fusion protein with N2Q, N58Q, R198A, and N288Q point mutations; and SEQ ID NO: 81, the amino acid sequence for the TTP4000 RAGE fusion protein with G59S, and R198A point mutations; Panels F through J show SEQ ID NOs: 92 through 111, which correspond to SEQ ID NOs: 62 through 81, respectively, with the exception that the C-terminal lysine has been removed; Panels K through O show SEQ ID NOs: 112 through 131, which correspond to SEQ ID NOs: 62 through 81, respectively, with the exception that there is an N-terminal pyroglutamic acid residue in place of a N-terminal glutamine residue; Panels P through T show SEQ ID NOs: 132 through 151, which correspond to SEQ ID NOs: 62 through 81, respectively, with the exception that the N-terminal glutamine has been removed; Panels U through Y show SEQ ID NOs: 152 through 171, which correspond to SEQ ID NOs: 132 through 151, respectively, with the exception that the C-terminal lysine has been removed; Panels Z through DD show SEQ ID NOs: 172 through 190 and also SEQ ID NO: 200, which correspond to SEQ ID NOs: 62 through 81, respectively, with the exception that there is an N-terminal pyroglutamic acid residue in place of a N-terminal glutamine residue and the C-terminal lysine has been removed; Panels EE-FF shows SEQ ID NOs: 58, 59, 60, 61, 82, and 201 which correspond to the amino acid sequence for the TTP4000 RAGE fusion protein (i.e., SEQ ID NO: 34) with N2Q, G59S, and R198A point mutations (SEQ ID NO: 58), and SEQ ID NO: 58 where the C-terminal lysine has been removed (SEQ ID NO: 59), SEQ ID NO: 58 with the exception that there is an N-terminal pyroglutamic acid residue in place of a N-terminal glutamine residue (SEQ ID NO: 60), SEQ ID NO: 58 with the exception that the N-terminal glutamine has been removed (SEQ ID NO: 61), SEQ ID NO: 61 where the C-terminal lysine has been removed (SEQ ID NO: 82), SEQ ID NO: 58 with the exception that there is an N-terminal pyroglutamic acid residue in place of a N-terminal glutamine residue and the C-terminal lysine has been removed (SEQ ID NO: 201); and SEQ ID NO: 225 corresponding to a RAGE fusion protein with one RAGE immunoglobulin domain and mutations at 3 glycosylation sites.

FIG. 7 shows alternate embodiments of DNA sequences encoding for the TTP4000 RAGE fusion protein of SEQ ID NO: 34 (TTP4000), wherein the RAGE fusion protein comprises a point mutation or mutations to remove a glycosylation site and/or a furin cleavage site: Panel A, SEQ ID NO: 221, a nucleotide sequence encoding for the TTP4000 RAGE fusion protein; Panel B, SEQ ID NO: 222, an alternate nucleotide sequence encoding for the TTP4000 RAGE fusion protein.

FIG. 8A shows the native amino acid signal sequence for human RAGE (SEQ ID NO: 83), as well as alternate embodiments of DNA sequences encoding for the native amino acid signal sequence for human RAGE (the native DNA signal sequence is SEQ ID NO: 84, a modified DNA signal sequence is SEQ ID NO: 85).

FIG. 8B shows various amino acid sequences for the immunoglobulin portion of the fusion protein. SEQ ID NO: 86 corresponds to SEQ ID NO: 38 with the exception that the C-terminal lysine has been removed; SEQ ID NO: 87 corresponds to SEQ ID NO: 40 with the exception that the C-terminal lysine has been removed; SEQ ID NO: 88 corresponds to SEQ ID NO: 40 with the exception that the first eleven N-terminal amino acids and a C-terminal lysine have been removed; SEQ ID NO: 89 corresponds to SEQ ID NO: 40 with the exception that the first eleven N-terminal amino acids have been removed, and that there is a point mutation from N to Q (shown in bold font); SEQ ID NO: 90 corresponds to SEQ ID NO: 42 with the exception that the first ten N-terminal amino acids have been removed; SEQ ID NO: 91 corresponds to SEQ ID NO: 42 with the exception that the first eleven N-terminal amino acids have been removed.

FIG. 8C—SEQ ID NO: 191 corresponds to SEQ ID NO: 46 with the exception of a N2Q point mutation; SEQ ID NO: 192 corresponds to SEQ ID NO: 46 with the exception of N2Q and N58Q point mutations; SEQ ID NO: 193 corresponds to SEQ ID NO: 46 with the exception a N58Q point mutation; SEQ ID NO: 194 corresponds to SEQ ID NO: 47 with the exception of a N2Q point mutation; SEQ ID NO: 195 corresponds to SEQ ID NO: 47 with the exception of a N2Q point mutation and the N-terminal pyroglutamic acid residue; SEQ ID NO: 196 forward primer sequence encoding mutation to remove glycosylation site in immunoglobulin polypeptide; SEQ ID NO: 197 reverse primer sequence with overlapping regions with SEQ ID NO: 196; SEQ ID NO: 198 forward primer sequence encoding mutation to remove furin cleavage site in RAGE polypeptide; SEQ ID NO: 199 reverse primer sequence with overlapping regions with SEQ ID NO: 198; SEQ ID NO: 223 corresponding to an 11 amino acid peptide comprising the hinge region of IgG1; and SEQ ID NO: 224 corresponding to a 10 amino acid peptide comprising the hinge region of IgG1.

FIG. 8D shows a nucleic acid sequence SEQ ID NO: 202 that can encode for the fusion proteins of SEQ ID NOS: 62, 92, 112, 132, 152, and 172.

FIG. 8E shows a nucleic acid sequence SEQ ID NO: 203 that can encode for the fusion proteins of SEQ ID NOS: 63, 93, 113, 133, 153, and 173.

FIG. 8F shows a nucleic acid sequence SEQ ID NO: 204 that can encode for the fusion proteins of SEQ ID NOS: 64, 94, 114, 134, 154, and 174.

FIG. 8G shows a nucleic acid sequence SEQ ID NO: 205 that can encode for the fusion proteins of SEQ ID NOS: 65, 95, 115, 135, 155, and 175.

FIG. 8H shows a nucleic acid sequence SEQ ID NO: 206 that can encode for the fusion proteins of SEQ ID NOS: 66, 96, 116, 136, 156, and 176.

FIG. 8I shows a nucleic acid sequence SEQ ID NO: 207 that can encode for the fusion proteins of SEQ ID NOS: 67, 97, 117, 137, 157, and 177.

FIG. 8J shows a nucleic acid sequence SEQ ID NO: 208 that can encode for the fusion proteins of SEQ ID NOS: 68, 98, 118, 138, 158, and 178.

FIG. 8K shows a nucleic acid sequence SEQ ID NO: 209 that can encode for the fusion proteins of SEQ ID NOS: 69, 99, 119, 139, 159, and 179.

FIG. 8L shows a nucleic acid sequence SEQ ID NO: 210 that can encode for the fusion proteins of SEQ ID NOS: 70, 100, 120, 140, 160, and 180.

FIG. 8M shows a nucleic acid sequence SEQ ID NO: 211 that can encode for the fusion proteins of SEQ ID NOS: 71, 101, 121, 141, 161, and 181.

FIG. 8N shows a nucleic acid sequence SEQ ID NO: 212 that can encode for the fusion proteins of SEQ ID NOS: 72, 102, 122, 142, 162, and 182.

FIG. 8O shows a nucleic acid sequence SEQ ID NO: 213 that can encode for the fusion proteins of SEQ ID NOS: 73, 103, 123, 143, 163, and 183.

FIG. 8P shows a nucleic acid sequence SEQ ID NO: 214 that can encode for the fusion proteins of SEQ ID NOS: 74, 104, 124, 144, 164, and 184.

FIG. 8Q shows a nucleic acid sequence SEQ ID NO: 215 that can encode for the fusion proteins of SEQ ID NOS: 75, 105, 125, 145, 165, and 185.

FIG. 8R shows a nucleic acid sequence SEQ ID NO: 216 that can encode for the fusion proteins of SEQ ID NOS: 76, 106, 126, 146, 166, and 186.

FIG. 8S shows a nucleic acid sequence SEQ ID NO: 217 that can encode for the fusion proteins of SEQ ID NOS: 77, 107, 127, 147, 167, and 187.

FIG. 8T shows a nucleic acid sequence SEQ ID NO: 219 that can encode for the fusion proteins of SEQ ID NOS: 78, 108, 128, 148, 168, and 188.

FIG. 8U shows a nucleic acid sequence SEQ ID NO: 219 that can encode for the fusion proteins of SEQ ID NOS: 79, 109, 129, 149, 169, and 189.

FIG. 8V shows a nucleic acid sequence SEQ ID NO: 220 that can encode for the fusion proteins of SEQ ID NOS: 80, 110, 130, 150, 170, and 190.

FIG. 9 shows differences in pharmacokinetic profiles for the RAGE fusion proteins of Batches 1-5 described herein. Plasma concentrations were measured over time periods from 0-336 hours (FIG. 9A) and 0-48 hours (FIG. 9B).

FIG. 10 shows mean pharmacokinetic parameters for the RAGE fusion proteins of Batches 1-5 described herein following intravenous administration to mice at 10 mg/kg.

FIG. 11 shows in vitro stability data for the RAGE fusion proteins of Batches 1-5 described below. FIG. 11A provides this data in tabular form, while FIG. 11B provides this data in graphic form.

FIG. 12 shows relative binding affinities for various mutant RAGE fusion proteins.

### DETAILED DESCRIPTION

Unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Also, the terms “portion” and “fragment” are used interchangeably to refer to parts of a polypeptide, nucleic acid, or other molecular construct.

“Polypeptide” and “protein” are used interchangeably herein to describe protein molecules that may comprise either partial or full-length proteins.

As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein.

The peptides, polypeptides and protein sequences disclosed herein use either the conventional 1 letter code, or the conventional 3 letter code for amino acids. For example, as used herein, the amino acids include the following non-limiting listing: glycine is represented as Gly or G; alanine represented as Ala or A; isoleucine represented as Ile or I; asparaginine represented as Asn or N; glutamine represented as Gln or Q; serine represented as Ser or S; threonine represented as Thr or T; protein represented as Pro or P; arginine represented as Arg or R; glutamate represented as Glu or E; lysine is represented as Lys or K; histidine is represented as His or H; leucine is represented as Leu or L; and pyroglutamic acid represented as pE.

As used herein, the term “upstream” refers to a residue that is N-terminal to a second residue where the molecule is a protein, or 5′ to a second residue where the molecule is a nucleic acid. Also as used herein, the term “downstream” refers to a residue that is C-terminal to a second residue where the molecule is a protein, or 3′ to a second residue where the molecule is a nucleic acid.

Unless defined otherwise, all technical and scientific terms used herein have the same meaning as commonly understood by one of ordinary skill in the art. Practitioners are particularly directed to Current Protocols in Molecular Biology (see e.g. Ausubel, F. M. et al., Short Protocols in Molecular Biology, 4^{th} Ed., Chapter 2, John Wiley & Sons, N.Y.) for definitions and terms of the art. Abbreviations for amino acid residues are the standard 3-letter and/or 1-letter codes used in the art to refer to one of the 20 common L-amino acids.

A “nucleic acid” is a polynucleotide such as deoxyribonucleic acid (DNA) or ribonucleic acid (RNA). The term is used to include single-stranded nucleic acids, double-stranded nucleic acids, and RNA and DNA made from nucleotide or nucleoside analogues.

The term “vector” refers to a nucleic acid molecule that may be used to transport a second nucleic acid molecule into a cell. In one embodiment, the vector allows for replication of DNA sequences inserted into the vector. The vector may comprise a promoter to enhance expression of the nucleic acid molecule in at least some host cells. Vectors may replicate autonomously (extrachromasomal) or may be integrated into a host cell chromosome. In one embodiment, the vector may comprise an expression vector capable of producing a protein derived from at least part of a nucleic acid sequence inserted into the vector.

As is known in the art, conditions for hybridizing nucleic acid sequences to each other can be described as ranging from low to high stringency. Generally, highly stringent hybridization conditions refer to washing hybrids in low salt buffer at high temperatures. Hybridization may be to filter bound DNA using hybridization solutions standard in the art such as 0.5M NaHPO₄, 7% sodium dodecyl sulfate (SDS), at 65° C., and washing in 0.25 M NaHPO₄, 3.5% SDS followed by washing 0.1×SSC/0.1% SDS at a temperature ranging from room temperature to 68° C. depending on the length of the probe. For example, a high stringency wash comprises washing in 6×SSC/0.05% sodium pyrophosphate at 37° C. for a 14 base oligonucleotide probe, or at 48° C. for a 17 base oligonucleotide probe, or at 55° C. for a 20 base oligonucleotide probe, or at 60° C. for a 25 base oligonucleotide probe, or at 65° C. for a nucleotide probe about 250 nucleotides in length. Nucleic acid probes may be labeled with radionucleotides by end-labeling with, for example, [γ-³²P]ATP, or incorporation of radiolabeled nucleotides such as [α-³²P]dCTP by random primer labeling. Alternatively, probes may be labeled by incorporation of biotinylated or fluorescein labeled nucleotides, and the probe detected using Streptavidin or anti-fluorescein antibodies.

As used herein, “small organic molecules” are molecules of molecular weight less than 2,000 Daltons that contain at least one carbon atom.

The term “fusion protein” refers to a protein or polypeptide that has an amino acid sequence derived from two or more proteins. The fusion protein may also include linking regions of amino acids between amino acid portions derived from separate proteins.

As used herein, “immunoglobulin peptides” may comprise an immunoglobulin heavy chain or a portion thereof. In one embodiment, the portion of the heavy chain may be the Fc fragment or a portion thereof. As used herein, the Fc fragment comprises the heavy chain hinge polypeptide, and the C_{H}2 and C_{H}3 domains of the heavy chain of an immunoglobulin, in either monomeric or dimeric form. Or, the C_{H}1 and Fc fragment may be used as the immunoglobulin polypeptide. The heavy chain (or portion thereof) or immunoglobulin polypeptide may be derived from or be a fragment of any one of the known heavy chain isotypes: e.g., IgG (γ), IgM (μ), IgD (δ), IgE (ε), or IgA (α). In addition, the heavy chain (or portion thereof) or immunoglobulin polypeptide may be derived from or be a fragment of any one of the known heavy chain subtypes: e.g., IgG1 (γ1), IgG2 (γ2), IgG3 (γ3), IgG4 (γ4), IgA1 (α1), IgA2 (α2), or mutations of these isotypes or subtypes that alter the biological activity. An example of biological activity that may be altered includes reduction of an isotype's ability to bind to some Fc receptors as for example, by modification of the hinge region.

The terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. In one embodiment, the percent identity of two sequences may be determined using GCG with a gap weight of 1, such that each amino acid gap is weighted as if it were a single amino acid mismatch between the two sequences.

As used herein, the term “conserved residues” refers to amino acids that are the same among a plurality of proteins having the same structure and/or function. A region of conserved residues may be important for protein structure or function. Thus, contiguous conserved residues as identified in a three-dimensional protein may be important for protein structure or function. To find conserved residues, or conserved regions of 3-D structure, a comparison of sequences for the same or similar proteins from different species, or of individuals of the same species, may be made.

As used herein, the term “homologue” means a polypeptide having a degree of homology or identity with the wild-type amino acid sequence. Homology comparisons can be conducted by eye, or more usually, with the aid of readily available sequence comparison programs. These commercially available computer programs can calculate percent homology between two or more sequences (e.g. Wilbur, W. J. and Lipman, D. J., 1983, Proc. Natl. Acad. Sci. USA, 80:726-730). For example, homologous sequences may be taken to include an amino acid sequences which in alternate embodiments are at least 70% identical, 75% identical, 85% identical, 90% identical, 95% identical, 96% identical, 97% identical, 98% identical, or 99% identical to each other.

As used herein, the term at least 90% identical thereto includes sequences that range from 90 to 99.99% identity to the indicated sequences and includes all ranges in between. Thus, the term at least 90% identical thereto includes sequences that are 91, 91.5, 92, 92.5, 93, 93.5, 94, 94.5, 95, 95.5, 96, 96.5, 97, 97.5, 98, 98.5, 99, 99.5 percent identical to the indicated sequence. Similarly the term at least 70% identical includes sequences that range from 70 to 99.99% identical, with all ranges in between. The determination of percent identity may be determined using the algorithms such as, but not limited to, those described herein.

As used herein, a polypeptide or protein “domain” comprises a region of a polypeptide or protein that comprises an independent unit. Domains may be defined in terms of structure, sequence and/or biological activity. In one embodiment, a polypeptide domain may comprise a region of a protein that folds in a manner that is substantially independent from the rest of the protein. Domains may be identified using domain databases such as, but not limited to PFAM, PRODOM, PROSITE, BLOCKS, PRINTS, SBASE, ISREC PROFILES, SAMRT, and PROCLASS.

As used herein, “immunoglobulin domain” is a sequence of amino acids that is structurally homologous, or identical to, a domain of an immunoglobulin. The length of the sequence of amino acids of an immunoglobulin domain may be any length. In one embodiment, an immunoglobulin domain may be less than 250 amino acids. In an example embodiment, an immunoglobulin domain may be about 80-150 amino acids in length. For example, the variable region, and the C_{H}1, C_{H}2, and C_{H}3 regions of an IgG are each immunoglobulin domains. In another example, the variable, the C_{H}1, C_{H}2, C_{H}3 and C_{H}4 regions of an IgM are each immunoglobulin domains.

As used herein, a “RAGE immunoglobulin domain” is a sequence of amino acids from RAGE protein that is structurally homologous, or identical to, a domain of an immunoglobulin. For example, a RAGE immunoglobulin domain may comprise the RAGE V-domain, the RAGE Ig-like C1-type 1 domain (“C1 domain”), or the RAGE Ig-like C2-type 2 domain (“C2 domain”).

As used herein, an “interdomain linker” comprises a polypeptide that joins two domains together. A hinge region is an example of an interdomain linker in an IgG.

As used herein, “directly linked” identifies a covalent linkage between two different groups (e.g., nucleic acid sequences, polypeptides, polypeptide domains) that does not have any intervening atoms between the two groups that are being linked.

As used herein, a “ligand binding domain” or “ligand binding site” comprises residues in a protein that directly interact with a ligand, or residues involved in positioning the ligand in close proximity to those residues that directly interact with the ligand. The interaction of residues in the ligand binding domain may be defined by the spatial proximity of the residues to a ligand in the model or structure. The term ligand binding domain includes homologues of a ligand binding domain, or portions thereof. In this regard, deliberate amino acid substitutions may be made in the ligand binding domain on the basis of similarity in polarity, charge, solubility, hydrophobicity, or hydrophilicity of the residues, as long as the binding specificity of the ligand binding domain is retained. A ligand binding domain may exist in one or more ligand binding domains of a protein or polypeptide.

As used herein, the term “interact” refers to a condition of proximity between a ligand or compound, or portions or fragments thereof, and a portion of a second molecule of interest. The interaction may be non-covalent, for example, as a result of hydrogen-bonding, van der Waals interactions, or electrostatic or hydrophobic interactions, or it may be covalent.

As used herein, a “ligand” refers to a molecule or compound or entity that interacts with a ligand binding domain, including substrates or analogues or parts thereof. As described herein, the term “ligand” may refer to compounds that bind to the protein of interest. A ligand may be an agonist, an antagonist, or a modulator. Or, a ligand may not have a biological effect. Or, a ligand may block the binding of other ligands thereby inhibiting a biological effect. Ligands may include, but are not limited to, small molecule inhibitors. These small molecules may include peptides, peptidomimetics, organic compounds and the like. Ligands may also include polypeptides and/or proteins.

As used herein, a “modulator compound” refers to a molecule which changes or alters the biological activity of a molecule of interest. A modulator compound may increase or decrease activity, or change the physical or chemical characteristics, or functional or immunological properties, of the molecule of interest. For RAGE, a modulator compound may increase or decrease activity, or change the characteristics, or functional or immunological properties of the RAGE, or a portion thereof. A modulator compound may include natural and/or chemically synthesized or artificial peptides, modified peptides (e.g., phosphopeptides), antibodies, carbohydrates, monosaccharides, oligosaccharides, polysaccharides, glycolipids, heterocyclic compounds, nucleosides or nucleotides or parts thereof, and small organic or inorganic molecules. A modulator compound may be an endogenous physiological compound or it may be a natural or synthetic compound. Or, the modulator compound may be a small organic molecule. The term “modulator compound” also includes a chemically modified ligand or compound, and includes isomers and racemic forms.

An “agonist” comprises a compound that binds to a receptor to form a complex that elicits a pharmacological response specific to the receptor involved.

An “antagonist” comprises a compound that binds to a biomolecule to form a complex that does not give rise to a substantial pharmacological response and can inhibit the biological response induced by an agonist. In some cases the biomolecule may be a receptor (e.g., RAGE). Also, in some cases the biomolecule may be an agonist, such that the antagonist may bind to the agonist and prevent the agonist from interacting with its target.

RAGE agonists may therefore bind to RAGE and stimulate RAGE-mediated cellular processes, and RAGE antagonists may inhibit RAGE-mediated processes from being stimulated by a RAGE agonist. For example, in one embodiment, the cellular process stimulated by RAGE agonists comprises activation of TNF-αt gene transcription.

The term “peptide mimetics” refers to structures that serve as substitutes for peptides in interactions between molecules (Morgan et al., 1989, Ann. Reports Med. Chem., 24:243-252). Peptide mimetics may include synthetic structures that may or may not contain amino acids and/or peptide bonds but that retain the structural and functional features of a peptide, or agonist, or antagonist. Peptide mimetics also include peptoids, oligopeptoids (Simon et al., 1972, Proc. Natl. Acad, Sci., USA, 89:9367); and peptide libraries containing peptides of a designed length representing all possible sequences of amino acids corresponding to a peptide, or agonist or antagonist of the invention.

The term “treating” or “treat” refers to improving a symptom of a disease or disorder or improving the subject's condition. The term “treatment” as used herein, refers to the full spectrum of treatments for a given disorder from which the subject is suffering, including alleviation of one symptom or most of the symptoms resulting from that disorder.

The term “cure” refers to the restoration of health in a subject, and/or the alleviation of all or substantially all of the symptoms of a disease or disorder in a subject.

The term “preventing the onset of a disorder” refers to impeding the development of the disorder in a subject that would have otherwise developed the disorder.

As used herein, the term “EC50” is defined as the concentration of an agent that results in 50% of a measured biological effect. For example, the EC50 of a therapeutic agent having a measurable biological effect may comprise the value at which the agent displays 50% of the biological effect.

As used herein, the term “IC50” is defined as the concentration of an agent that results in 50% inhibition of a measured effect. For example, the IC50 of an antagonist of RAGE binding may comprise the value at which the antagonist reduces ligand binding to the ligand binding domain of RAGE by 50%.

As used herein, the phrase “therapeutically effective amount” shall mean that amount of a drug or pharmaceutical agent that will elicit the therapeutic response of a subject that is being sought.

In these methods, factors which may influence what constitutes a therapeutically effective amount include, but are not limited to, the size and weight of the subject, the biodegradability of the therapeutic agent, the activity of the therapeutic agent, the size of the effected area, as well as its bioavailability. The phrase includes amounts which, as compared to a corresponding subject who has not received such amount, results in improved treatment, healing, or amelioration of a side effect, or a decrease in the rate of advancement of a disease or disorder.

The term “pharmaceutically acceptable carrier” as used herein may refer to compounds and compositions that are suitable for use in human or animal subjects, as for example, for therapeutic compositions administered for the treatment of a RAGE-mediated disorder or disease.

The term “pharmaceutical composition” is used herein to denote a composition that may be administered to a mammalian host, e.g., orally, parenterally, topically, by inhalation spray, intracerebroventricularly, intrathecally, intranasally, rectally, or any other form of administration route as described herein, in unit dosage formulations containing conventional non-toxic carriers, diluents, adjuvants, vehicles and the like.

The term “parenteral” as used herein, includes subcutaneous injections, intravenous, intramuscular, intracisternal injection, or infusion techniques.

As used herein “rejection” refers to the immune or inflammatory response on tissue that leads to destruction of cells, tissues or organs, or that leads to damage to cells, tissues, or organs. The rejected cells, tissue, or organ may be derived from the same subject that is mounting the rejection response, or may be transplanted from a different subject into the subject that is displaying rejection.

As used herein, the term “cell” refers to the structural and functional units of a mammalian living system that each comprise an independent living system. As is known in the art, cells include a nucleus, cytoplasm, intracellular organelles, and a cell wall which encloses the cell and allows the cell to be independent of other cells.

As used herein, the term “tissue” refers to an aggregate of cells that have a similar structure and function, or that work together to perform a particular function. A tissue may include a collection of similar cells and the intercellular substances surrounding the cells. Tissues include, but are not limited to, muscle tissue, nerve tissue, and bone.

As used herein an “organ” refers to a fully differentiated structural and functional unit in an animal that is specialized for some specific function. An organ may comprise a group of tissues that perform a specific function or group of functions. Organs include, but are not limited to, the heart, lungs, brain, eye, stomach, spleen, pancreas, kidneys, liver, intestines, skin, uterus, bladder, and bone.

A “stable” formulation is one in which the RAGE fusion protein therein essentially retains its physical and chemical stability and biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 1 week to 1 month, at which time stability is measured. For example, the extent of aggregation following lyophilization and storage can be used as an indicator of RAGE fusion protein stability. For example, a “stable” formulation may be one wherein less than about 10% and preferably less than about 5% of the RAGE fusion protein is present as an aggregate in the formulation. In other embodiments, an increase in aggregate formation following lyophilization and storage of the lyophilized formulation can be determined. For example, a “stable” lyophilized formulation may be one wherein the increase in aggregate in the lyophilized formulation is less than about 5% or less than about 3%, when the lyophilized formulation is incubated at 40° C. for at least one week. In other embodiments, stability of the RAGE fusion protein formulation may be measured using a biological activity assay such as a binding assay as described herein.

A “reconstituted” formulation is one which has been prepared by dissolving a lyophilized RAGE fusion protein formulation in a diluent such that the RAGE fusion protein is dispersed and/or dissolved in the reconstituted formulation. The reconstituted formulation may be suitable for administration (e.g. parenteral administration) to a patient to be treated with the fusion protein and, in certain embodiments of the invention, may be one which is suitable for subcutaneous administration.

By “isotonic” it is meant that the formulation of interest has an osmotic pressure from about 240 to about 340 mOsm/kg. In an embodiment, an isotonic formulation is one having an osmotic pressure that is essentially the same as human blood (285-310 mOsm/kg). Isotonicity can be measured using a vapor pressure or a freezing point depression type osmometer.

A “lyoprotectant” is a molecule which, when combined with a RAGE fusion protein, significantly prevents or reduces chemical and/or physical instability of the protein upon lyophilization and subsequent storage. Exemplary lyoprotectants include sugars such as sucrose or trehalose; a polyol such as sugar alcohols, e.g. erythritol, arabitol, xylitol, sorbitol, and mannitol; or combinations thereof. In an embodiment, the lyoprotectant may comprise a sugar. In another embodiment, the lyoprotectant may comprise a non-reducing sugar. In a further embodiment, the lyoprotectant may comprise a non-reducing sugar such as sucrose. The lyoprotectant may be added to the pre-lyophilized formulation in a “lyoprotecting amount” which means that, following lyophilization of the protein in the presence of the lyoprotecting amount of the lyoprotectant, the RAGE fusion protein essentially retains its physical and chemical stability and biological activity upon lyophilization and storage.

The “diluent” for a lyophilized formulation herein is one which is pharmaceutically acceptable (safe and non-toxic for administration to a human) and is useful for the preparation of a reconstituted formulation. Exemplary diluents include sterile water, bacteriostatic water for injection (BWFI), a pH buffered solution (e.g. phosphate-buffered saline), sterile saline solution, Ringer's solution or dextrose solution. In an embodiment, the diluent provides a reconstituted formulation suitable for injection. In another embodiment, where the diluent provides a reconstituted formulation suitable for injection, the diluent may comprise water for injection (WFI).

A “preservative” for a reconstituted formulation is a compound which can be added to the diluent or to the reconstituted formulation to essentially reduce bacterial action in the reconstituted formulation. In an embodiment, the amount of preservative may be added in an amount useful to facilitate the production of a multi-use reconstituted formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, allyl parabens such as methyl or propyl paraben, catechol, resorcinol, cyclohexanol, 3-pentanol, and m-cresol.

A “bulking agent” for a lyophilized formulation is a compound which adds mass to the lyophilized mixture and contributes to the physical structure of the lyophilized cake (e.g. facilitates the production of an essentially uniform lyophilized cake which maintains an open pore structure). Exemplary bulking agents include, but are not limited to, mannitol, glycine, and xorbitol.

### RAGE Fusion Proteins

Embodiments of the present invention comprise fusion proteins, nucleic acids encoding for such fusion proteins, methods of making such fusion proteins, and methods of use of such fusion proteins.

For example, in certain embodiments, the fusion proteins of the present invention comprise a Receptor for Advanced Endproducts (RAGE) polypeptide, wherein the RAGE polypeptide comprises a fragment of a mammalian wild type RAGE peptide, and wherein there is at least one point mutation in the RAGE polypeptide portion of the fusion protein relative to the wild type RAGE peptide. In an embodiment, the fusion protein may comprise a RAGE polypeptide and an immunoglobulin polypeptide, wherein the RAGE polypeptide comprises a fragment of a mammalian wild type RAGE peptide and wherein there is at least one point mutation in the RAGE polypeptide portion of the fusion protein relative to the wild type RAGE peptide and/or the immunoglobulin portion of the RAGE fusion protein.

For example, in certain embodiments, the present invention comprises a fusion protein comprising a RAGE polypeptide linked to an immunoglobulin polypeptide, wherein the RAGE polypeptide comprises a fragment of a mammalian RAGE having a ligand binding domain, and wherein there is at least one mutation in at least one of the RAGE polypeptide or the immunoglobulin polypeptide relative to the wild-type sequence of the fusion protein, wherein the mutation removes and/or alters at least one of a glycosylation site or an enzyme cleavage site.

The RAGE polypeptide may be from any mammal. In certain embodiments, the RAGE polypeptide is a human RAGE polypeptide. Also, the immunoglobulin polypeptide may be from any mammal. In certain embodiments, the immuoglobulin polypeptide is a human immunoglobulin polypeptide.

The point mutation may be selected to provide a beneficial effect in the properties of the RAGE fusion protein. As described in more detail herein, for each of the various embodiments of the RAGE fusion proteins and/or methods of use or making such proteins, the RAGE fusion protein may be modified to improve the properties of the molecule. For example, the RAGE fusion protein of the invention may comprise improved ligand binding as compared to RAGE fusion proteins that do not have at least one mutation relative to the wild-type sequence in at least one of the RAGE polypeptide or the immunoglobulin polypeptide, wherein the mutation removes and/or alters at least one of a glycosylation site or an enzyme cleavage site. Additionally or alternatively, the RAGE fusion protein of the invention may comprise improved stability as compared to RAGE fusion proteins that do not have at least one mutation relative to the wild-type sequence in at least one of the RAGE polypeptide or the immunoglobulin polypeptide, wherein the mutation removes and/or alters at least one of a glycosylation site or an enzyme cleavage site. Additionally or alternatively, the RAGE fusion protein of the invention may comprise an improved pharmacokinetic profile as compared to RAGE fusion proteins that do not have at least one mutation relative to the wild-type sequence in at least one of the RAGE polypeptide or the immunoglobulin polypeptide, wherein the mutation removes and/or alters at least one of a glycosylation site or an enzyme cleavage site.

For example, in certain embodiments, the point mutation changes the sequence in the wild-type RAGE polypeptide present at a glycosylation site. In this way, the type and/or extent of glycosylation may be modified.

The glycosylation site may be one of various types of glycosylation sites that are found in proteins. For example, in certain embodiments, the glycosylation recognition site is NXS or NXT. In certain embodiments, X is not proline. Thus, in certain embodiments, the point mutation changes the sequence in the wild-type RAGE polypeptide from NIT to QIT and/or from NGS to QGS, and/or from NGS to NSS, and/or from NST to QST to remove and/or alter at least one glycosylation site. In some embodiments, the glycosylation site is located in the RAGE portion of the RAGE fusion protein. For example, the glycosylation site may, in certain embodiments, be located within the ligand binding site or the ligand binding domain. Additionally and/or alternatively, the glycosylation site is located in the immunoglobulin portion of the RAGE fusion protein. Or, other glycosylation sites that may exist in the RAGE fusion protein may be targeted.

In certain embodiments, the enzyme cleavage site is a furin cleavage site, such that the point mutation changes the sequence in the wild-type RAGE polypeptide present at a recognition site for furin cleavage of the RAGE polypeptide. For example, in alternate embodiments, the point mutation changes the sequence in the wild-type RAGE polypeptide from one of: (i) PRHRALR to PRHAALR; (ii) PRHRALR to PRHKALR; (iii) PRHRALR to PRHRALA; (iv) PRHRALR to PRHRALK; (v) PRHRALR to PRHRALH; (vi) PRHRALR to PRHRALT; (vii) PRHRALR to PRHHALR; or (viii) PRHRALR to PRHTALR to alter and/or remove a furin cleavage site.

In certain embodiments, the RAGE polypeptide portion of the fusion protein does not include an N-terminal leader peptide. For example, in certain embodiments, the RAGE polypeptide portion of the fusion protein does not include an N-terminal leader peptide consisting of amino acids 1-23 of the wild-type RAGE. Or, in certain embodiments, the RAGE polypeptide portion of the fusion protein does not include an N-terminal leader peptide consisting of amino acids 1-22 of the wild-type RAGE polypeptide. Or, in other embodiments, the RAGE polypeptide portion of the fusion protein does not include an N-terminal leader peptide consisting of amino acids 1-24 of the wild-type RAGE polypeptide.

In certain embodiments, the at least one mutation comprises at least one of following: (i) N2Q; (ii) N58Q; (iii) G59S; (iv) N2Q and N58Q; or (v) N2Q and G59S of a human RAGE polypeptide processed such that the RAGE polypeptide portion of the fusion protein does not include an N-terminal leader peptide consisting of amino acids 1-23 of the wild-type RAGE polypeptide. For example, in this numbering system, the RAGE fusion proteins correspond to the amino acid sequence of the RAGE fusion protein of SEQ ID NO: 34 (a RAGE fusion protein having two RAGE immunoglobulin-like domains as described in more detail below), wherein the signal sequence of amino acids 1-23 is not included and the RAGE fusion protein comprises a point mutation or mutations to remove and/or alter a glycosylation site and/or a furin cleavage site based on number that begins with amino acid 24 relative to the full-length wild-type RAGE sequence (SEQ ID NO: 1). Also, in this numbering system, the RAGE fusion proteins correspond to the amino acid sequence of the RAGE fusion protein of SEQ ID NO: 37 (a RAGE fusion protein having one RAGE immunoglobulin-like domains as described in more detail below), wherein the signal sequence of amino acids 1-23 is not included and the RAGE fusion protein comprises a point mutation or mutations to remove and/or alter a glycosylation site and/or a furin cleavage site based on number that begins with amino acid 24 relative to the full-length wild-type RAGE sequence (SEQ ID NO: 1). For example, SEQ ID NO: 62 provides an amino acid sequence for a RAGE fusion protein having two RAGE domains, wherein the first RAGE domain includes an N2Q point mutation which would correspond to N25Q in the full-length RAGE wild-type sequence of SEQ ID NO: 1. Similarly, SEQ ID NO: 63 provides an amino acid sequence for a RAGE fusion protein having two RAGE domains with an N58Q point mutation which would correspond to N81Q in the full-length wild-type sequence. Similar sequences may be generated with RAGE fusion proteins having a single RAGE domain (e.g., SEQ ID NO: 57, 37, 36, and 35) or three domains. For example, as described in more detail herein, SEQ ID NO: 225 is a fusion protein having a single RAGE immunoglobulin-like domain corresponding to amino acids 24 to 136 of RAGE, with mutations in positions 2 (i.e., corresponding to amino acid 25 of the full-length RAGE protein), 58 and 173 of the fusion protein.

As described in detail herein, the RAGE polypeptide may be a fragment of full-length RAGE. SEQ ID NO: 1 provides an amino acid sequence for full-length RAGE.

In certain embodiments, the human RAGE polypeptide is processed such that the RAGE polypeptide portion of the fusion protein does not include an N-terminal leader peptide consisting of amino acids 1-23 of the wild-type RAGE polypeptide. In this embodiment, the RAGE polypeptide comprising at least one mutation at a glycosylation site may comprise the sequence as set forth in at least one of: (i) SEQ ID NO: 8, SEQ ID NO: 16, or SEQ ID NO: 20; or (ii) SEQ ID NO: 8, SEQ ID NO: 16, or SEQ ID NO: 20 having the N-terminal glutamine cyclized to form pyroglutamic acid.

Similarly, in certain embodiments, the mutation to remove the furin cleavage site comprises at least one of R195A, R195K, R195T, R195H, R198A, R198K, R198H, R198T of a human RAGE polypeptide that does not include an N-terminal leader peptide consisting of amino acids 1-23 of the wild-type RAGE polypeptide. In this embodiment, the RAGE polypeptide comprising at least one mutation at a furin cleavage site may comprise the sequence as set forth in SEQ ID NO: 20 or SEQ ID NO: 20 having the N-terminal glutamine cyclized to form pyroglutamic acid. This site is not present in the shorter RAGE fusion proteins of SEQ ID NO: 57, 37, 36, and 35.

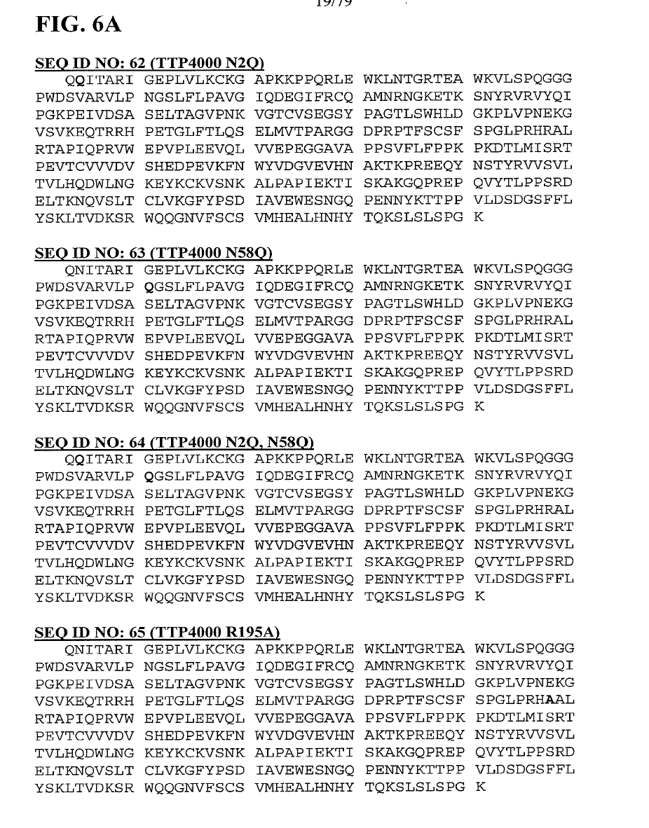

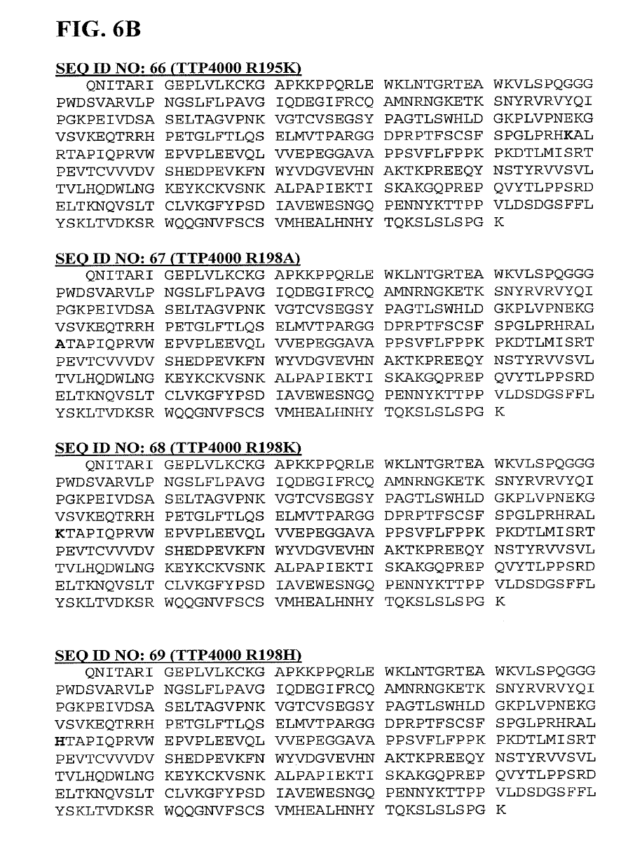

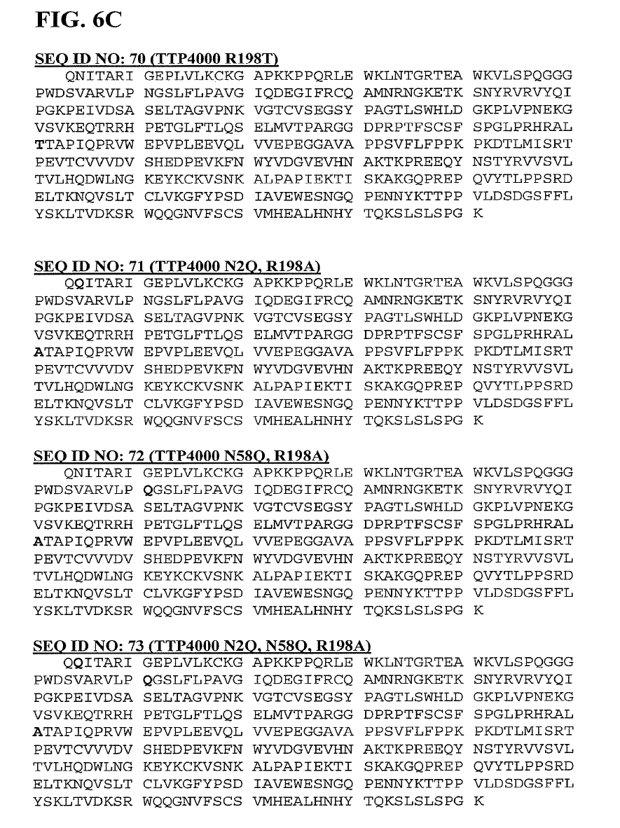

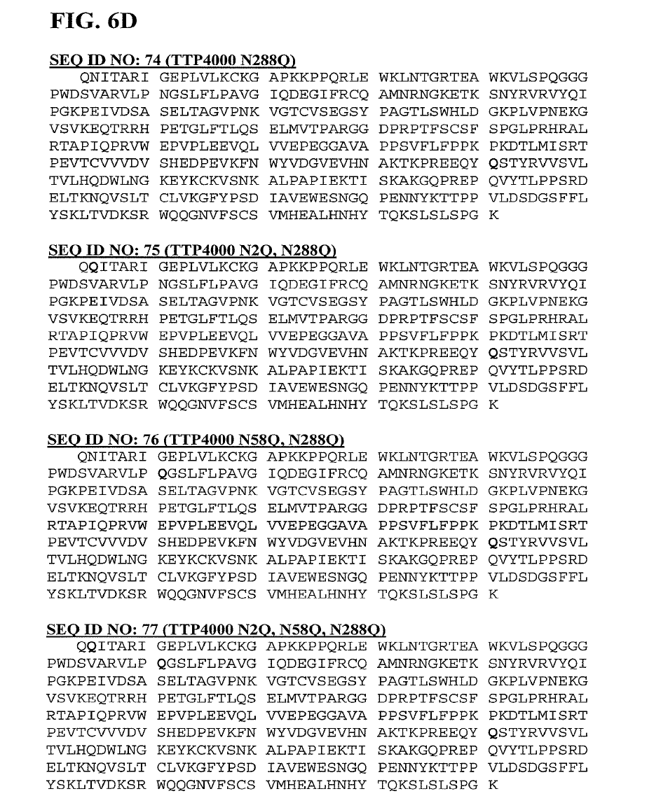

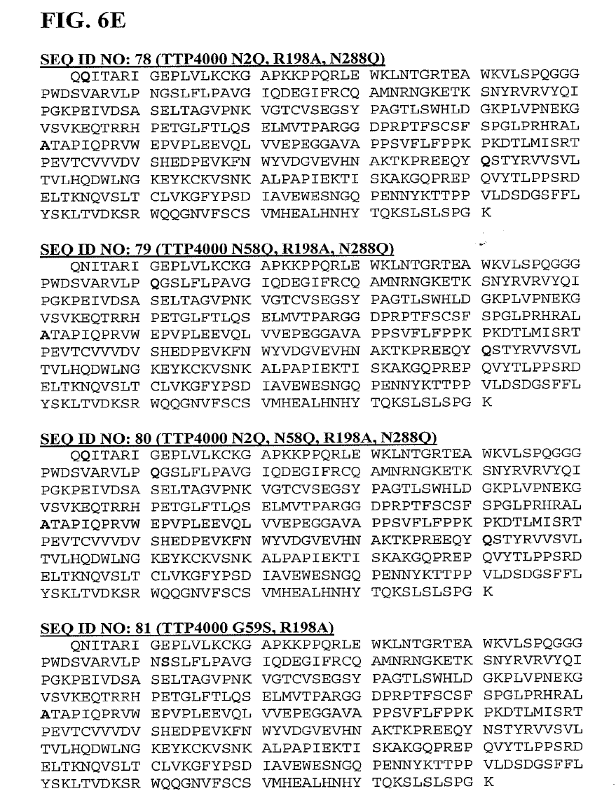

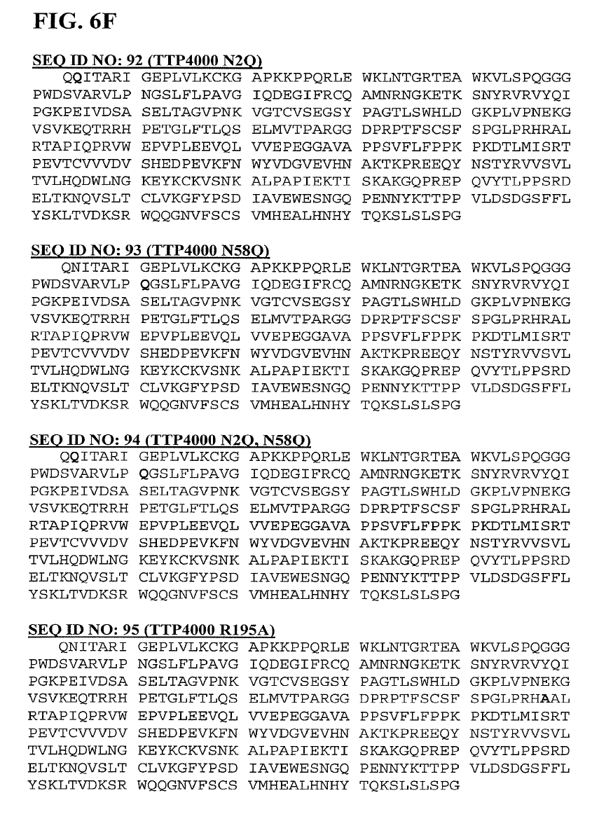

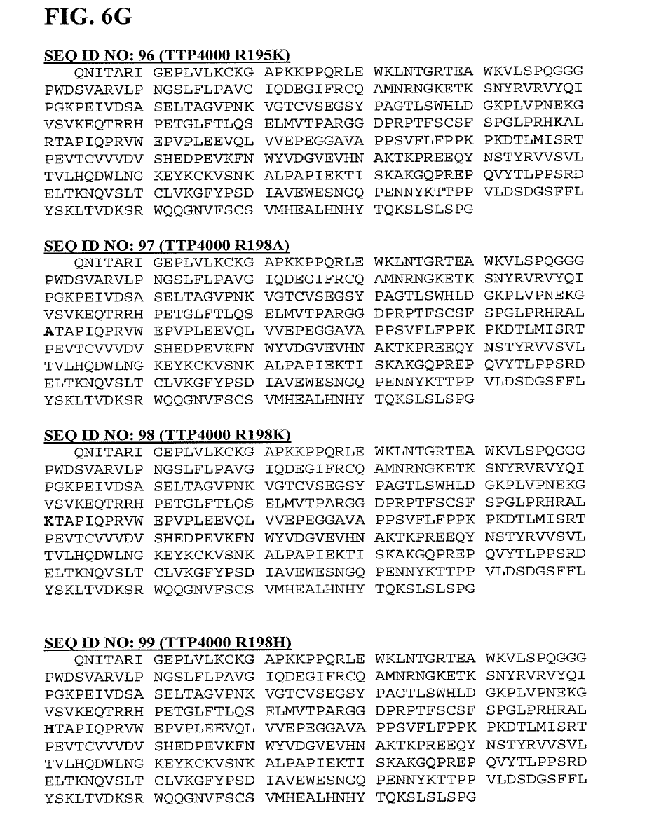

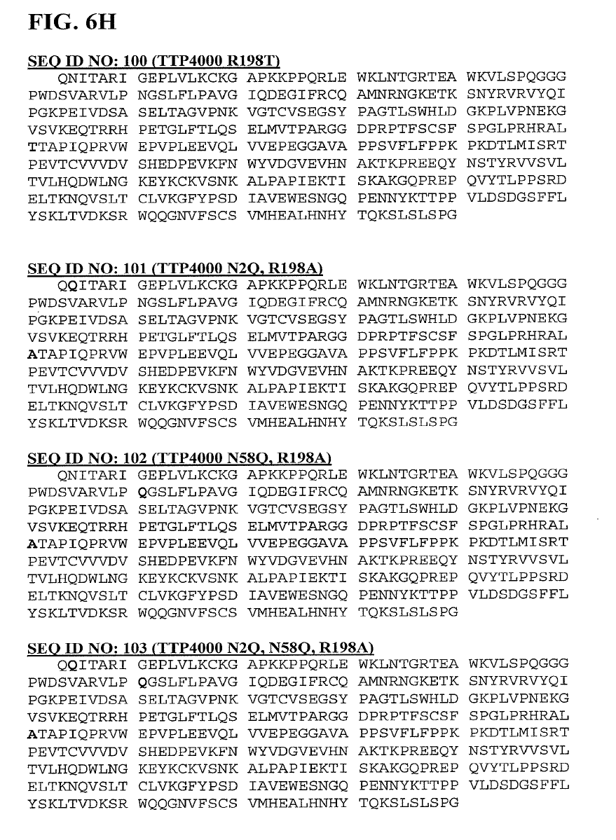

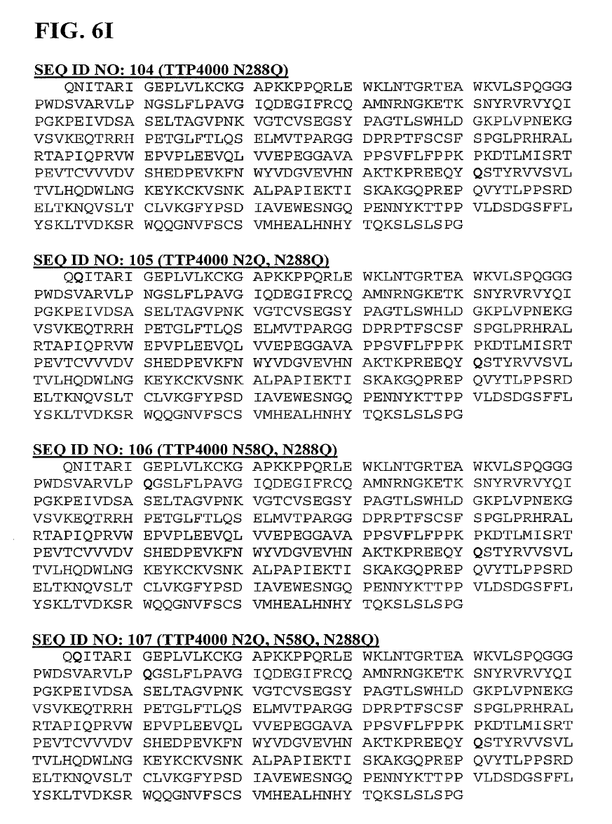

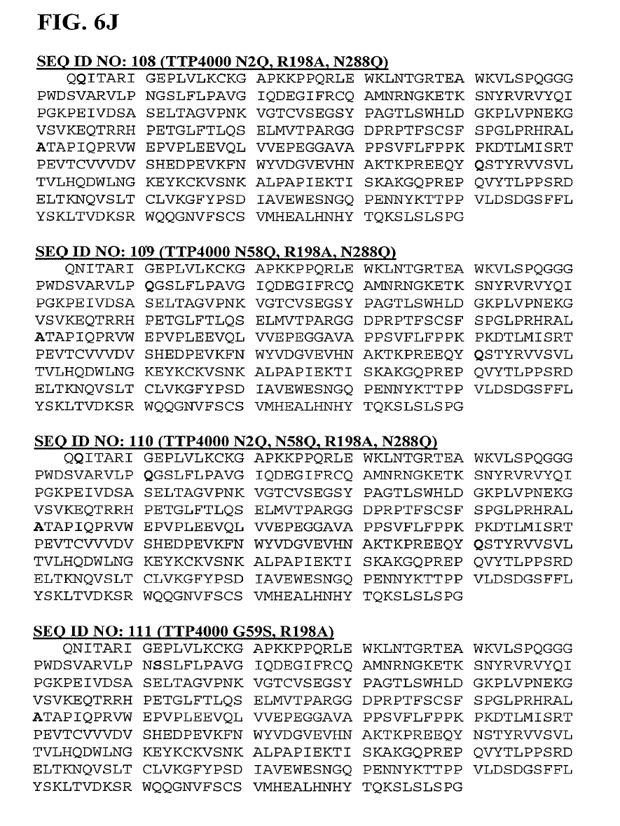

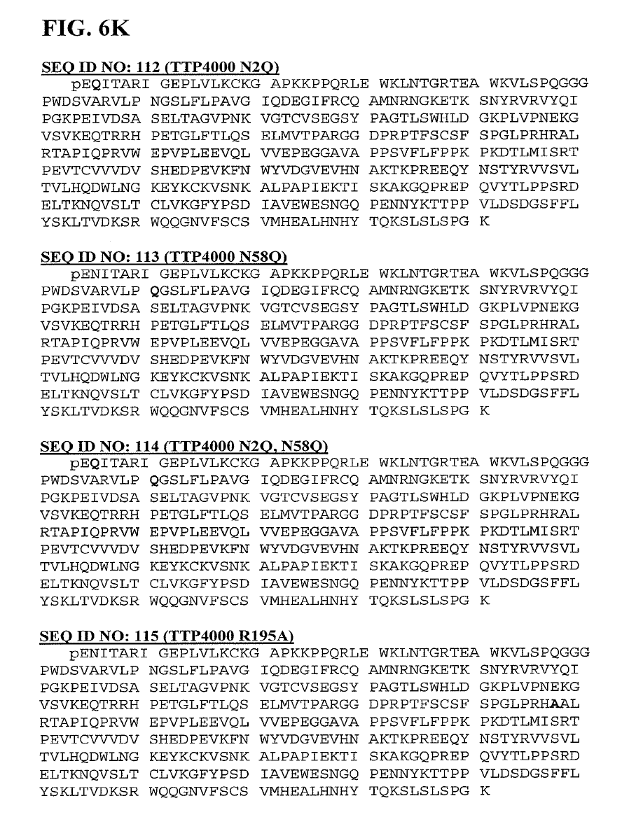

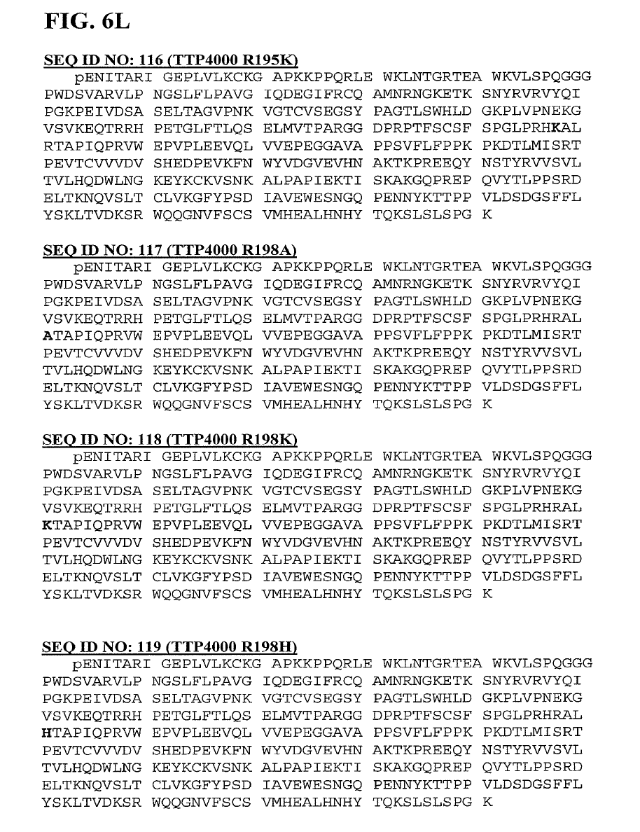

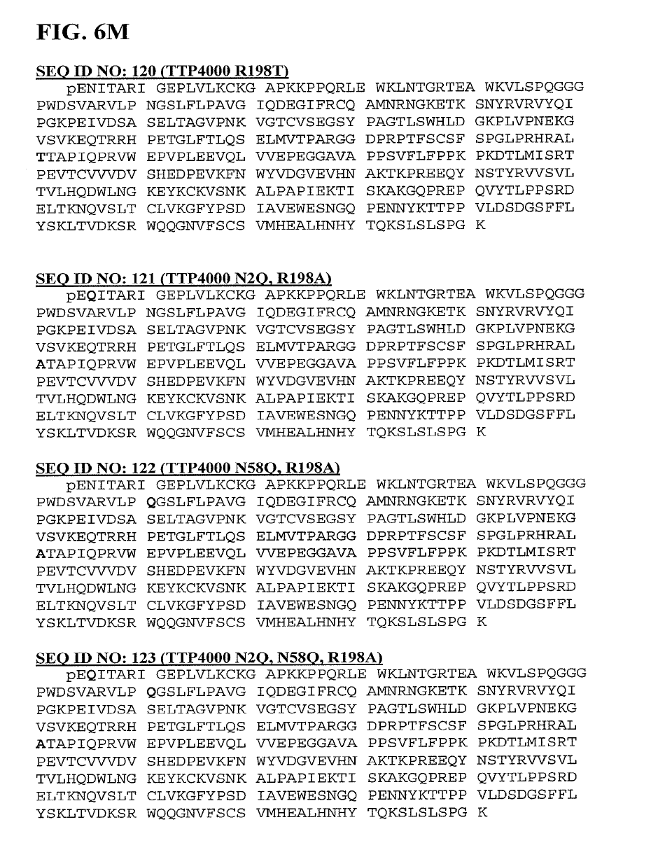

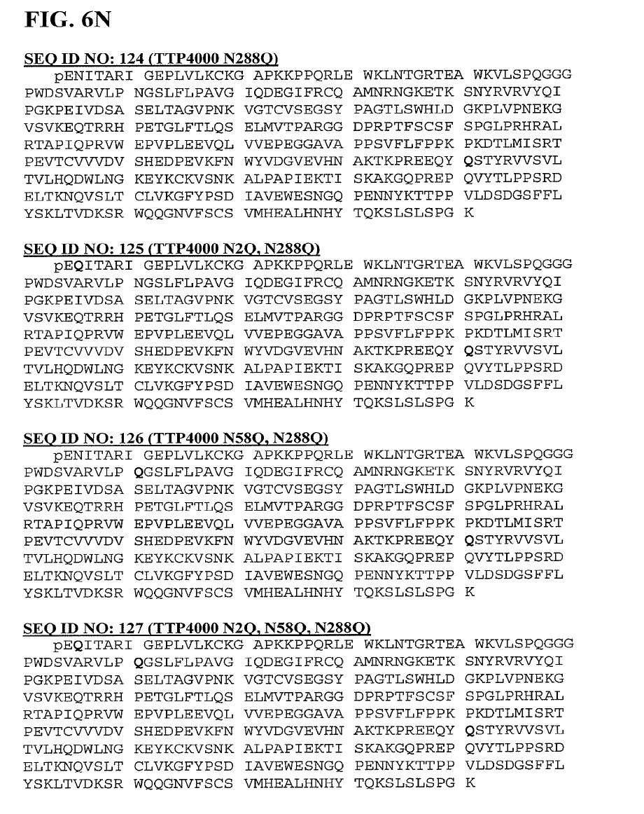

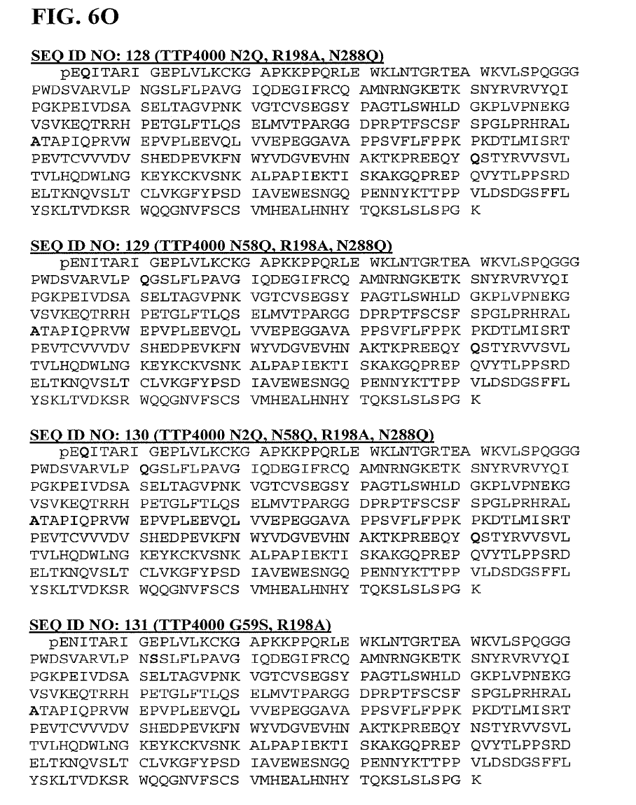

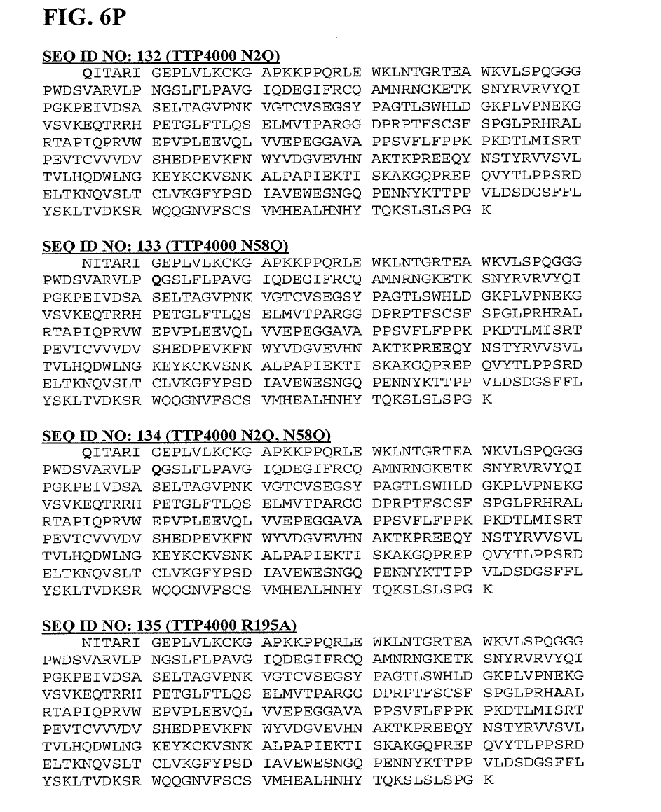

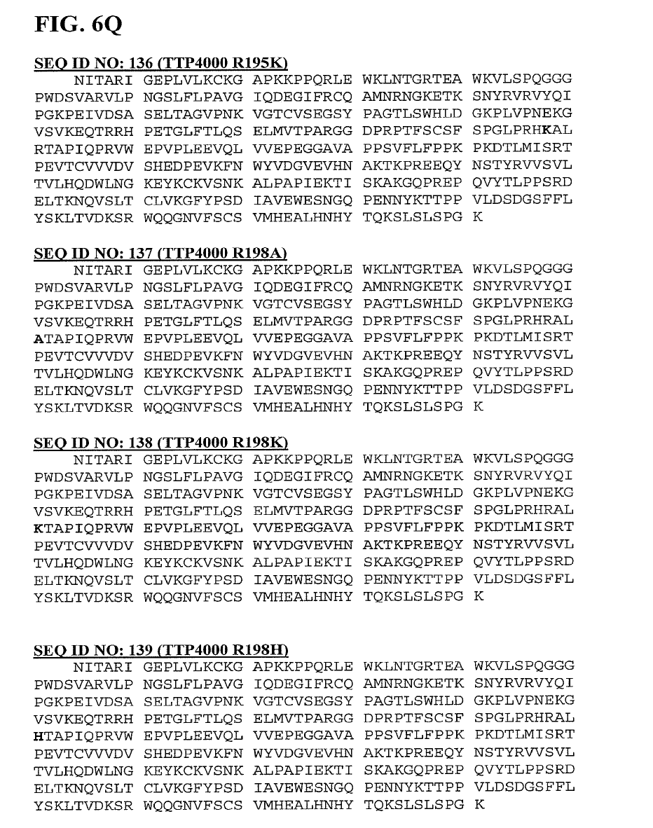

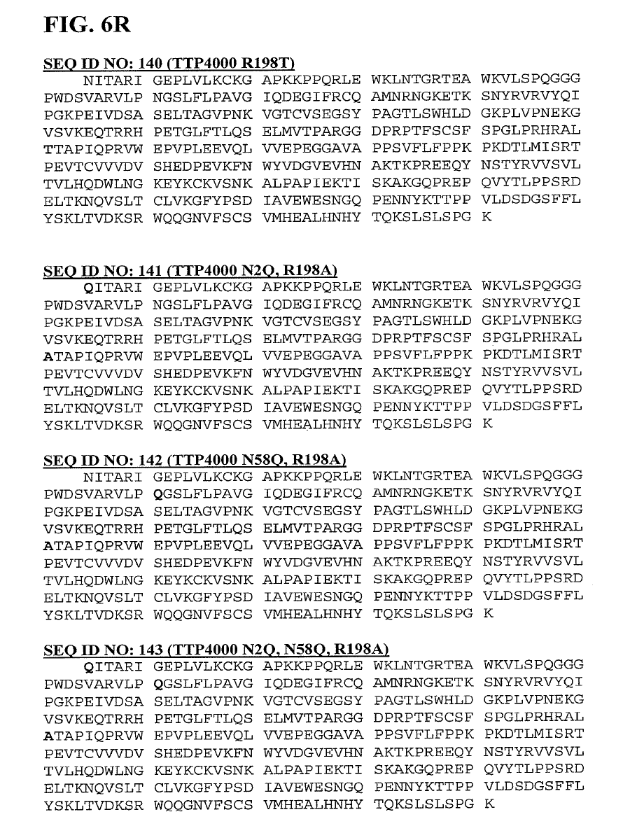

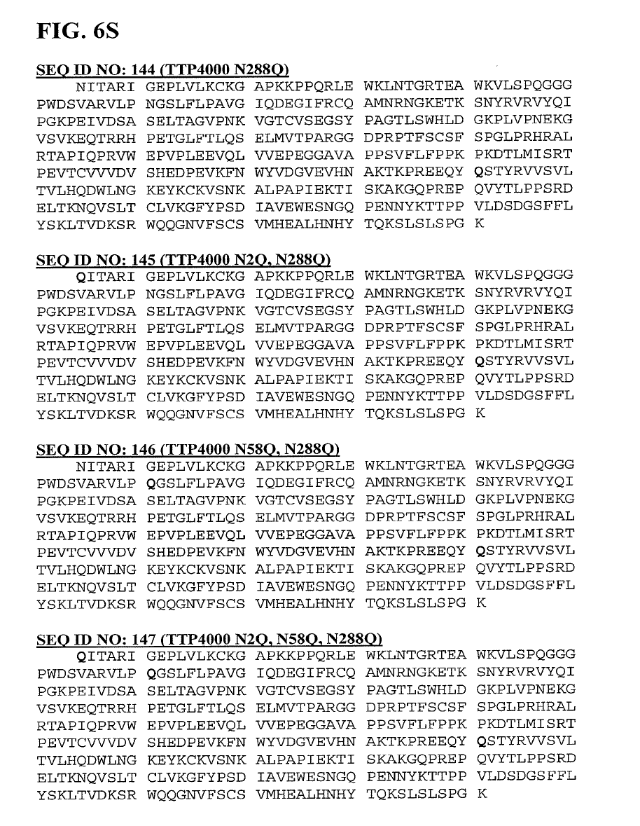

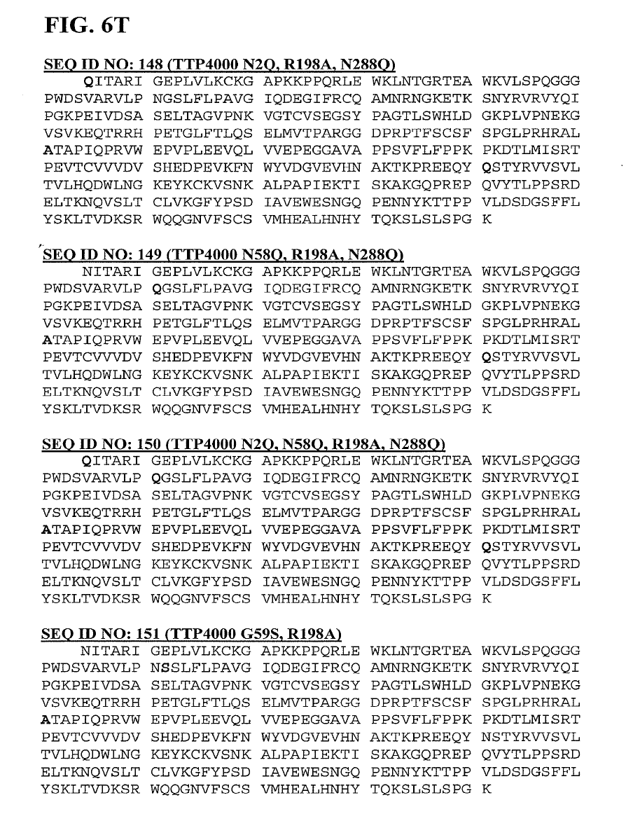

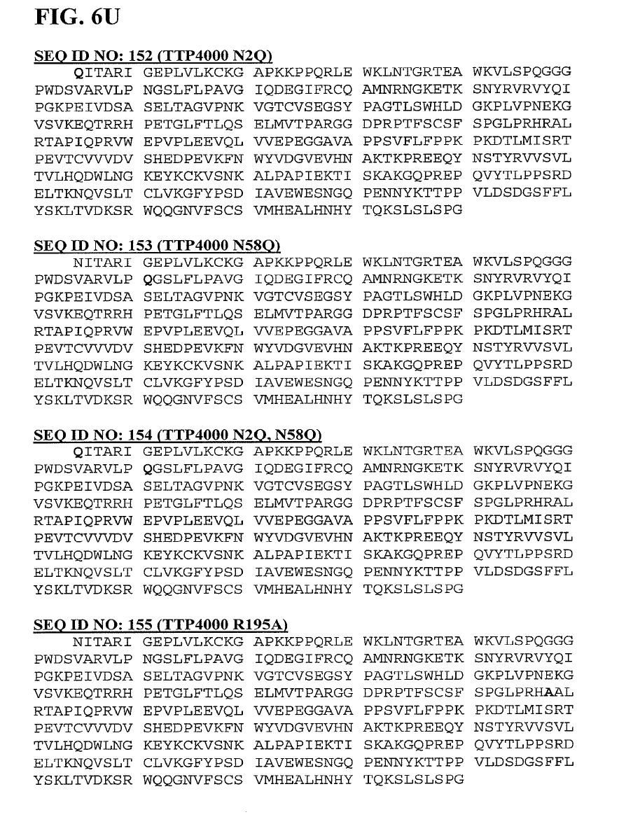

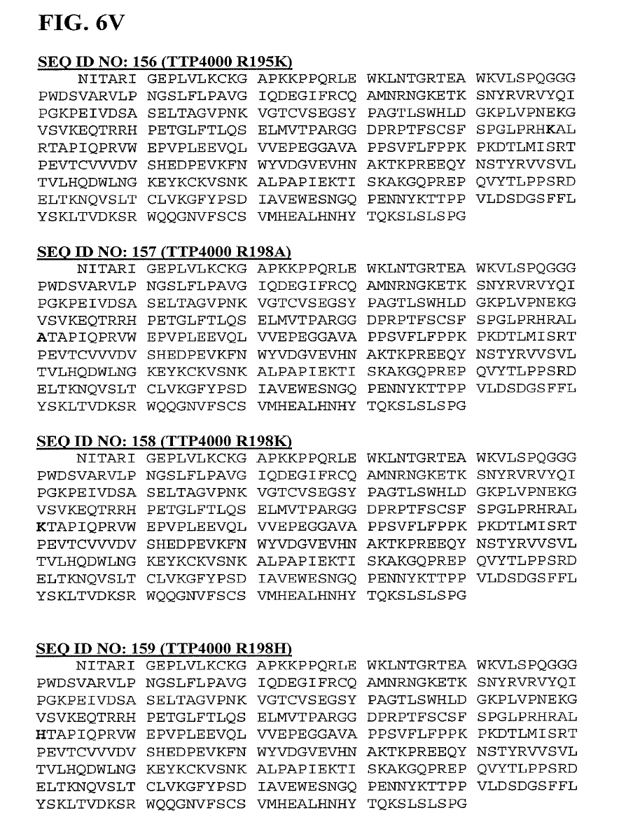

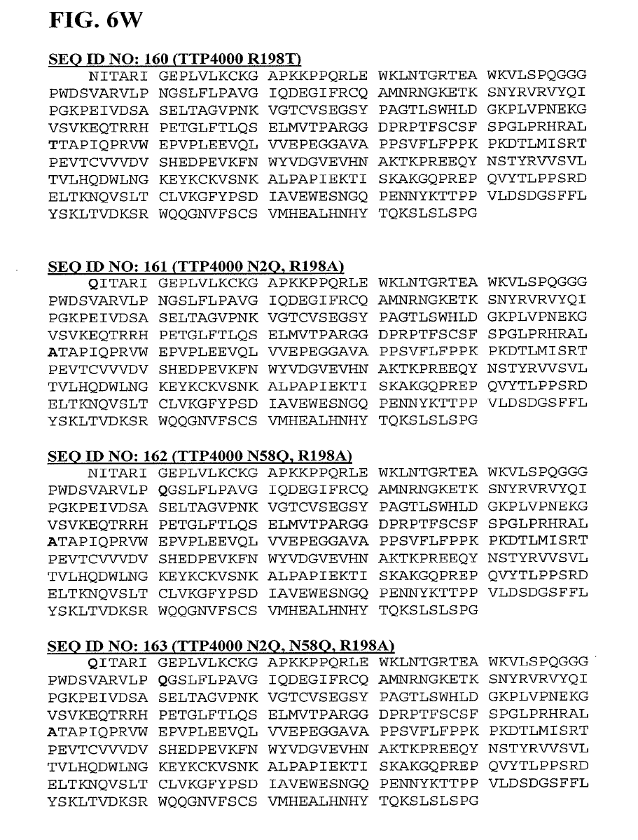

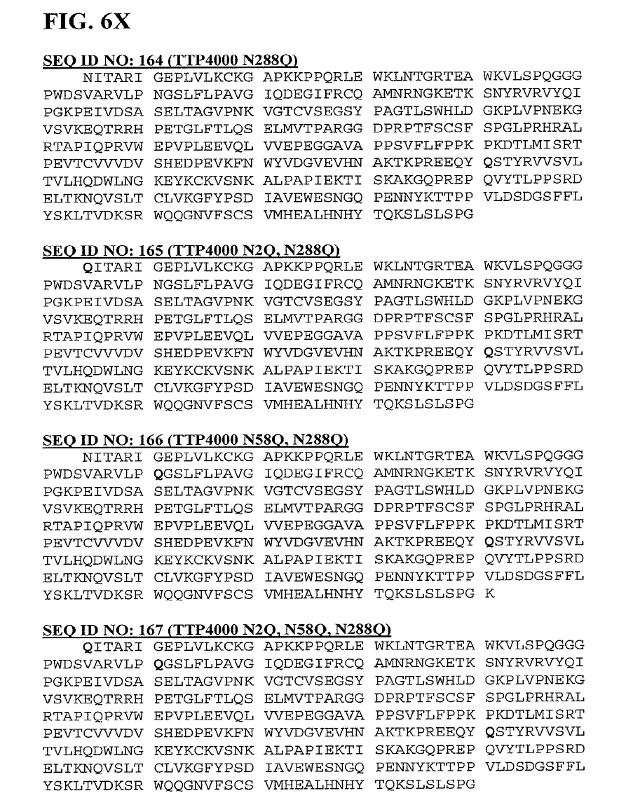

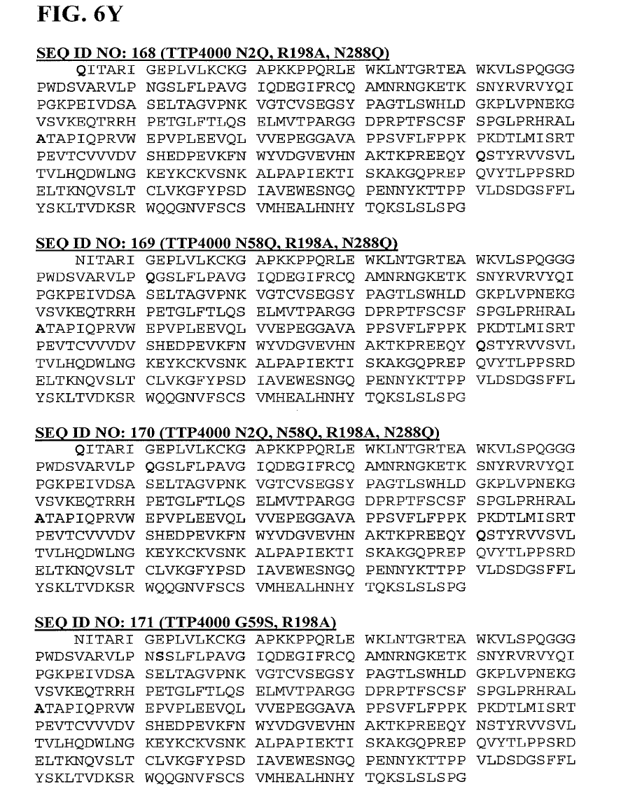

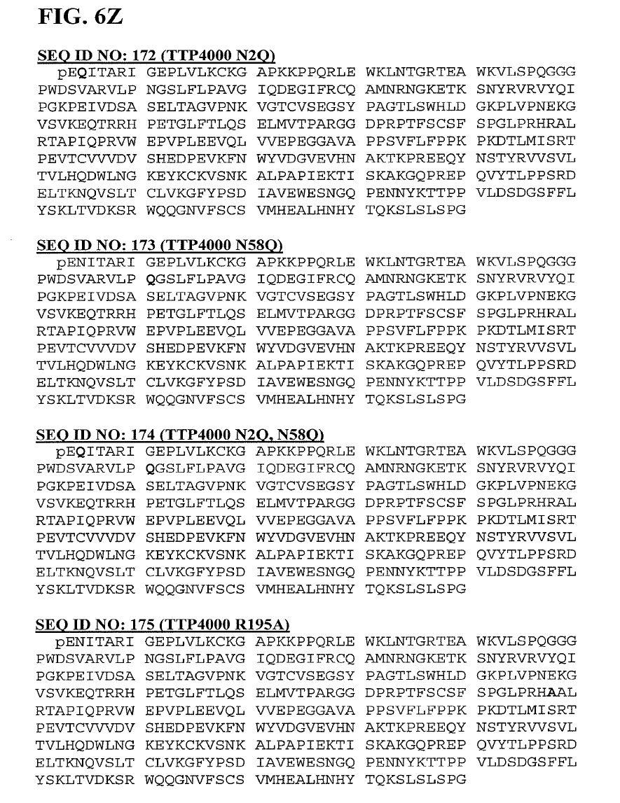

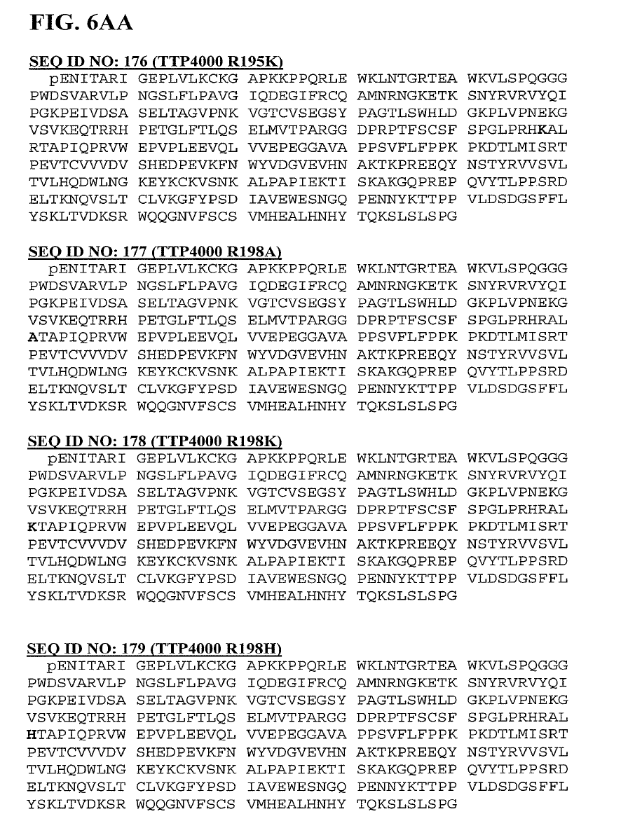

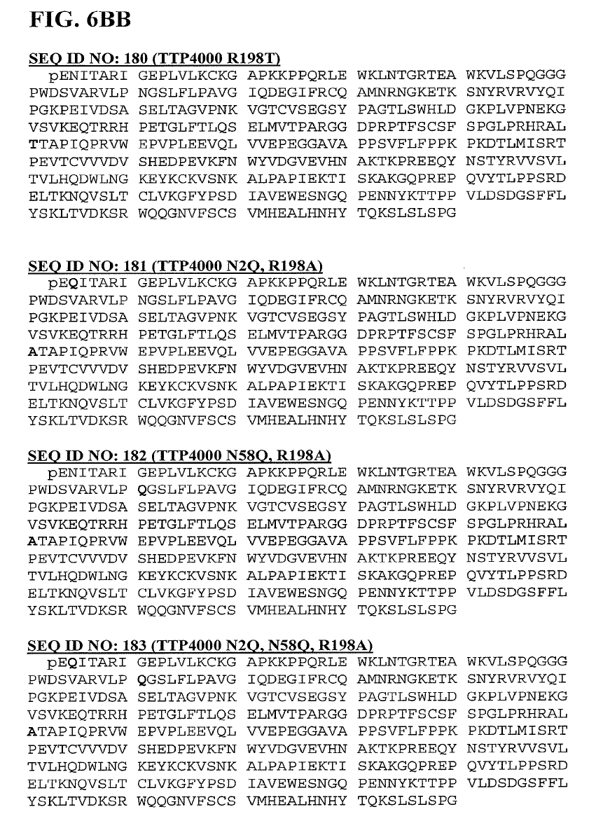

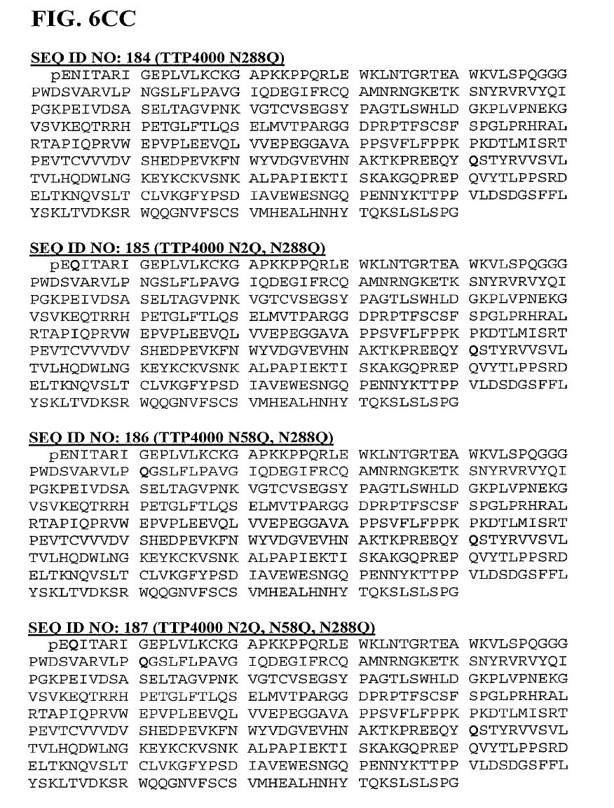

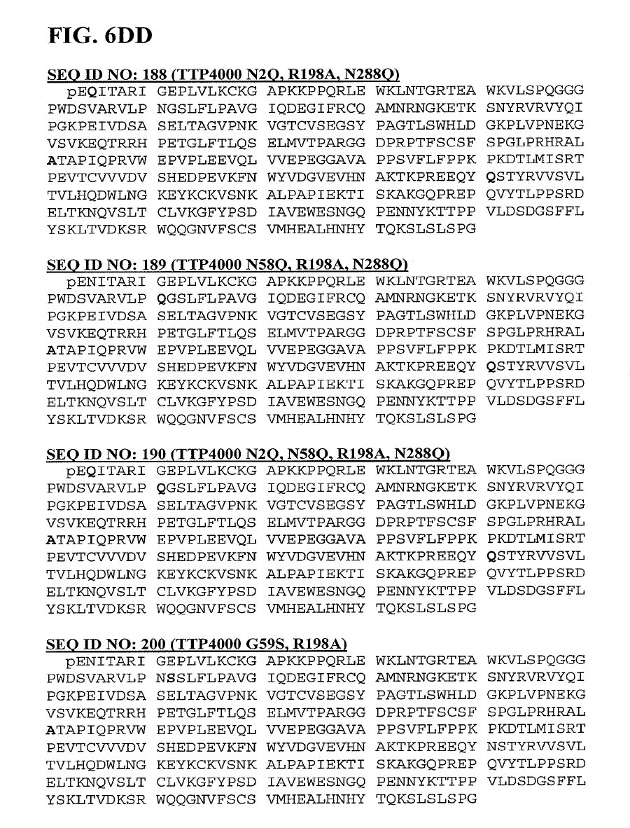

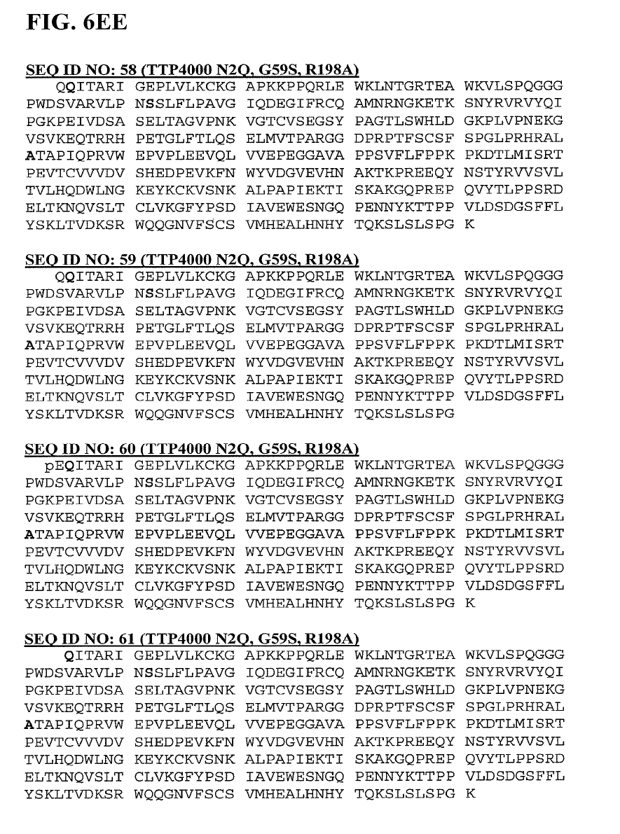

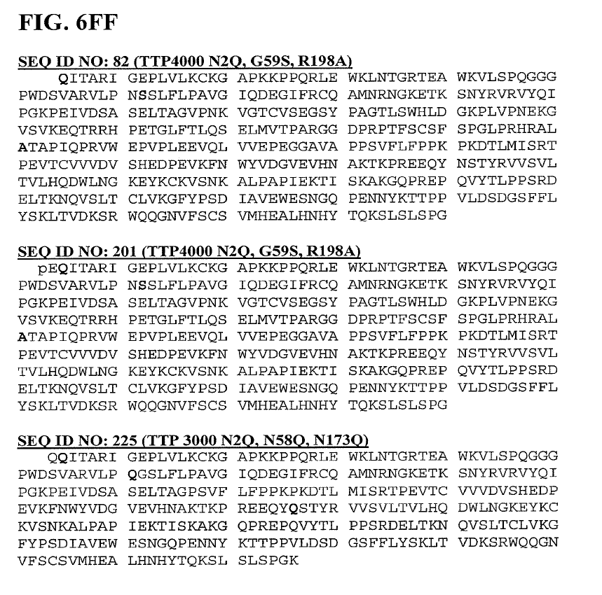

As described in more detail herein, for certain embodiments, the immunoglobulin portion of the RAGE fusion protein is modified to improve the properties of the molecule. For example, in certain embodiments the RAGE fusion protein comprises a mutation at a glycosylation site at position 288 of a human RAGE fusion protein having two RAGE immunoglobulin-like domains (e.g., SEQ ID NO: 34) wherein the RAGE polypeptide portion of the fusion protein does not include an N-terminal leader peptide consisting of amino acids 1-23 of the wild-type RAGE polypeptide. In certain embodiments, the mutation changes a glycosylation site having the amino acid sequence of NST to QST. In an embodiment, the mutation comprises N288Q relative to the RAGE fusion sequence presented as SEQ ID NO: 34. A similar protein may be made in a RAGE fusion protein having only one RAGE domain (TTP3000) (e.g., SEQ ID NO: 37). In this case, the immunoglobulin mutation may be found at position 173. An example of a RAGE fusion protein having a single RAGE domain and with mutations at position 2, 58 and 173 is provided as SEQ ID NO: 225 (FIG. 6FF). It will be understood by those in the art that the mutations described for a fusion protein having two RAGE domains may be made in analogous positions in a RAGE fusion protein having only one RAGE domain or, in proteins having three RAGE domains. The furin cleavage site is not present in the shorter RAGE fusion proteins having only one domain as illustrated in SEQ ID NOs: 57, 37, 36, and 35, but would be present in a wild-type RAGE fusion protein having three RAGE immunoglobulin-like domains.

Also, as described herein, for each of the embodiments of the RAGE fusion proteins, nucleic acid constructs, and/or other compositions, methods and systems of the invention, the immunoglobulin polypeptide may comprise a C_{H}2 domain or a fragment of a C_{H}2 domain. In an embodiment, the C_{H}2 domain or fragment of a C_{H}2 domain does not include at least a portion of the hinge region. In certain embodiments, the C_{H}2 domain or fragment of a C_{H}2 domain is linked via its C-terminus to the N-terminus of a C_{H}3 domain or a fragment of a C_{H}3 domain of an immunoglobulin polypeptide.

The immunoglobulin domain may be derived from various immunoglobulins as is known in the art. In certain embodiments, the immunoglobulin comprises a human IgG. Thus, in certain embodiments, the C_{H}2 and C_{H}3 domains of the immunoglobulin comprises SEQ ID NO: 38, or SEQ ID NO: 38 without the C-terminal lysine (i.e., SEQ ID NO: 86), or SEQ ID NO: 38 without the N-terminal proline, or SEQ ID NO: 38 without the N-terminal proline and the terminal lysine (SEQ ID NO: 88). In an embodiment, the C_{H}2 domain comprises SEQ ID NO: 90 or 91. In an embodiment, the portion of the hinge region that is not included in the C_{H}2 domain (or fragment thereof) has the sequence as set forth in SEQ ID NO: 223 or SEQ ID NO: 224. SEQ ID NO: 89 provides an illustrative example of the C_{H}2 and C_{H}3 domains of a human IgG immunoglobulin mutated at a position that corresponds to position 288 of SEQ ID NO: 34.

Thus, in certain embodiments, the present invention may comprise a RAGE fusion protein comprising a RAGE polypeptide comprising an amino acid sequence as set forth in any one of SEQ ID NOs: 62 to 81, SEQ ID NOs: 92 to 190, or SEQ ID NOs: 58-61, 82, 200 or 201. Also, in certain embodiments, the present invention may comprise a RAGE fusion protein comprising a RAGE polypeptide having an amino acid sequence as set forth in any one of SEQ ID NOs: 62-81, SEQ ID NOs: 92-190, or SEQ ID NOs: 58-61, 82, 200 or 201.

The RAGE fusion proteins of the present invention may form non-covalent associations with each other. Thus, in certain embodiments, the fusion protein of the invention is in the form of a monomer, a dimer, a trimer, a tetramer, or a mixture thereof.

For example, in some embodiments, the present invention provides a fusion protein comprising a RAGE polypeptide and an immunoglobulin polypeptide, wherein: (a) the RAGE polypeptide comprises a fragment of a mammalian wild type RAGE polypeptide comprising a RAGE ligand binding domain, and wherein the RAGE polypeptide comprises a point mutation or mutations at one or more of residues relative to the wild type RAGE polypeptide to remove and/or alter a glycosylation site; and (b) the immunoglobulin polypeptide comprises at least a fragment of a C_{H}2 domain and a C_{H}3 domain of an immunoglobulin. In an embodiment, the C_{H}2 domain or fragment thereof is operably linked to the C_{H}3 domain. In other embodiments, the present invention provides a fusion protein comprising a RAGE polypeptide and an immunoglobulin polypeptide, wherein: (a) the RAGE polypeptide comprises a fragment of a mammalian wild type RAGE polypeptide comprising a RAGE ligand binding domain, and wherein the RAGE polypeptide comprises a point mutation or mutations at one or more of residues relative to the wild type RAGE polypeptide to remove and/or alter a furin cleavage site; and (b) the immunoglobulin polypeptide comprises at least a fragment of a C_{H}2 domain and a C_{H}3 domain of an immunoglobulin. In an embodiment, the C_{H}2 domain or fragment thereof is operably linked to the C_{H}3 domain.

In yet other embodiments, the present invention provides a fusion protein comprising a RAGE polypeptide and an immunoglobulin polypeptide, wherein: (a) the RAGE polypeptide comprises a fragment of a mammalian wild type RAGE polypeptide comprising a RAGE ligand binding domain, and wherein the RAGE fusion protein comprises: (i) a point mutation or mutations at one or more of residues relative to the wild type RAGE polypeptide and/or the immunoglobulin to remove and/or alter a glycosylation site, and/or (ii) a point mutation or mutations at one or more of residues relative to the wild type RAGE peptide to remove and/or alter a furin cleavage site; and (b) the immunoglobulin polypeptide comprises at least a fragment of a C_{H}2 domain and a C_{H}3 domain of an immunoglobulin. In an embodiment, the C_{H}2 domain or fragment thereof is operably linked to the C_{H}3 domain. As noted herein, in certain embodiments, and as described in more detail below, the immunoglobulin polypeptide comprises a fragment of a C_{H}2 domain which does not include at least a portion of the hinge region. Also, in certain embodiments, the hinge region, or portion thereof that is not included as part of the C_{H}2 domain has or comprises the sequence as set forth in SEQ ID NO: 223 or SEQ ID NO: 224.

Mutation of the wild type RAGE peptide in a fusion protein of the present invention may increase metabolic stability, pharmacokinetic half life relative, and/or the binding affinity relative to wild type RAGE fusion protein (i.e., a RAGE fusion protein without one or more point mutations to remove either a glycosylation site or furin cleavage site). In certain embodiments, the RAGE fusion proteins of the present invention may bind to RAGE ligands (such as S100b) with affinities in the high nanomolar to low micromolar range. Also, in some embodiments, the RAGE fusion proteins of the present invention may have greater affinity for a RAGE ligand than a corresponding wild type RAGE fusion protein. For example, in certain embodiments, the fusion proteins of the present invention having at least one point mutation in the RAGE polypeptide portion to remove a glycosylation site at N2 and/or N58 may have increased the binding affinity for s100b than a similar wild type RAGE fusion protein and may have at least double the binding affinity.

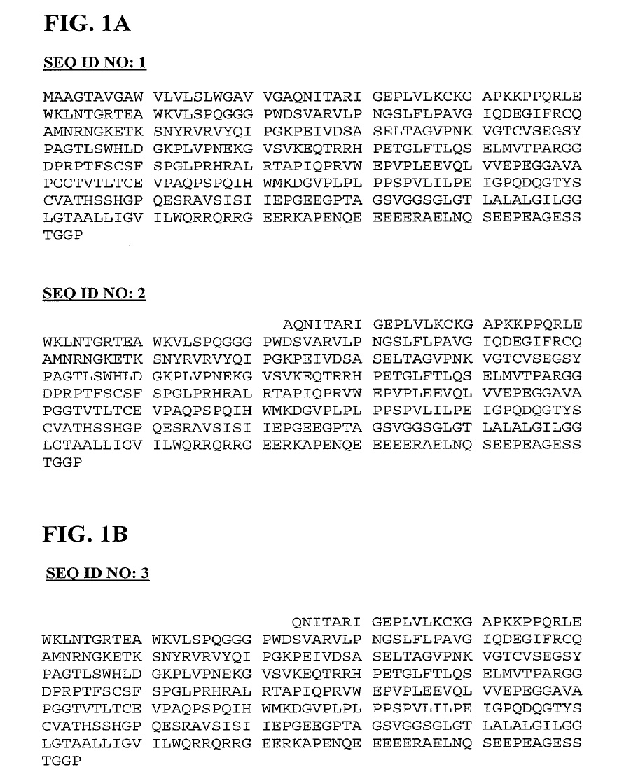

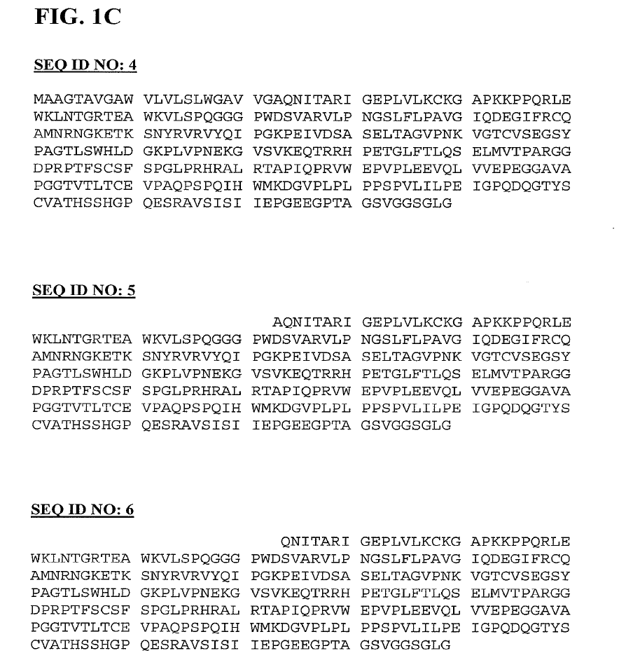

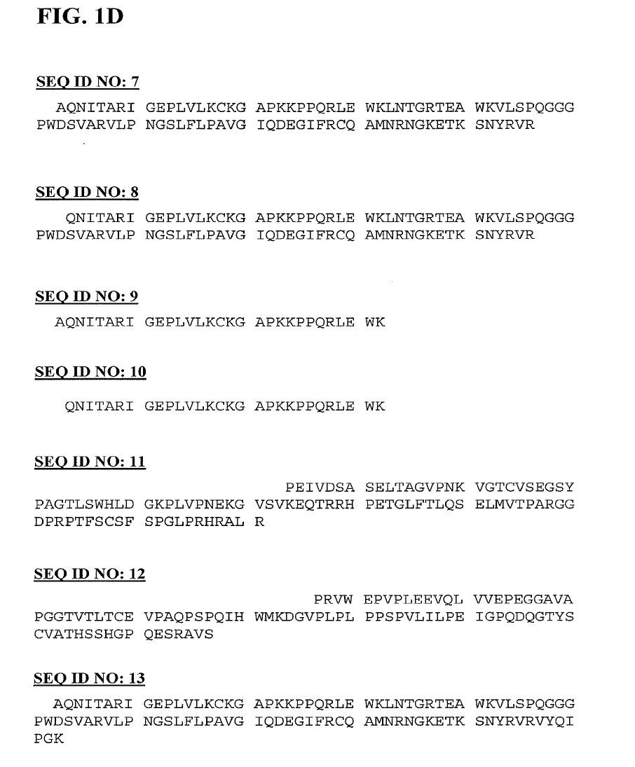

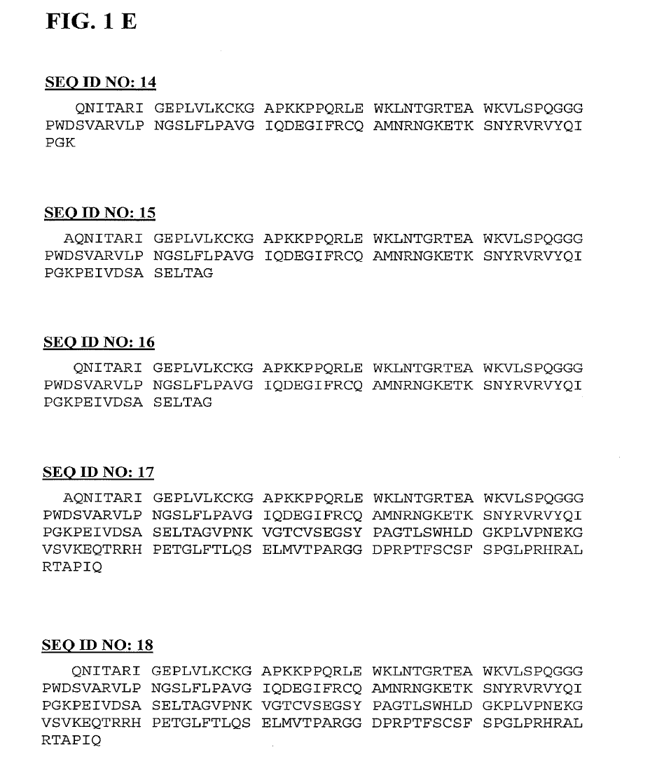

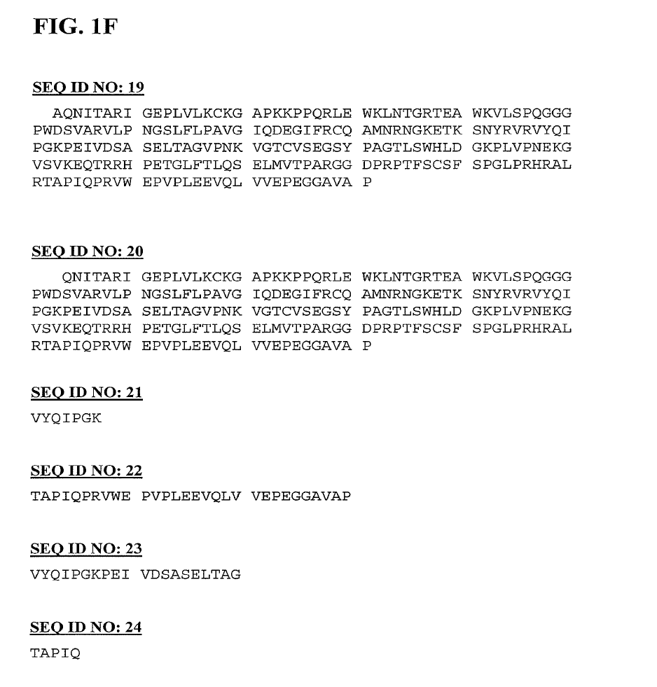

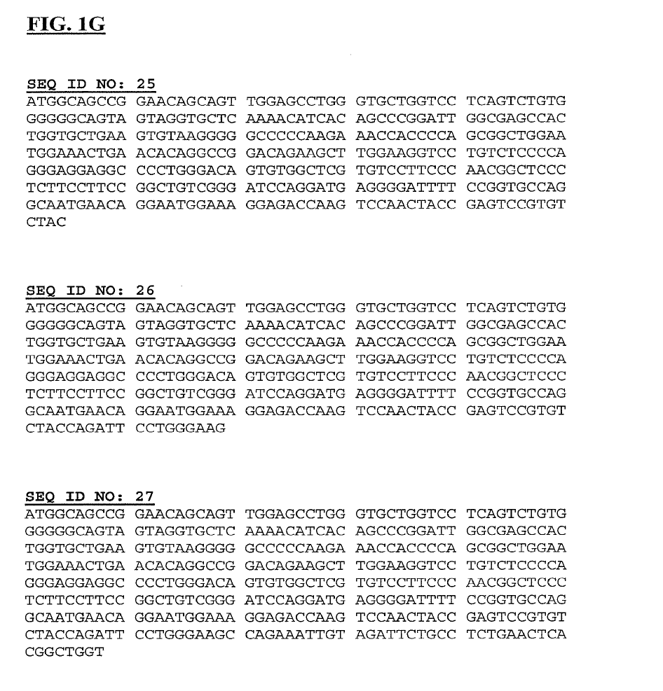

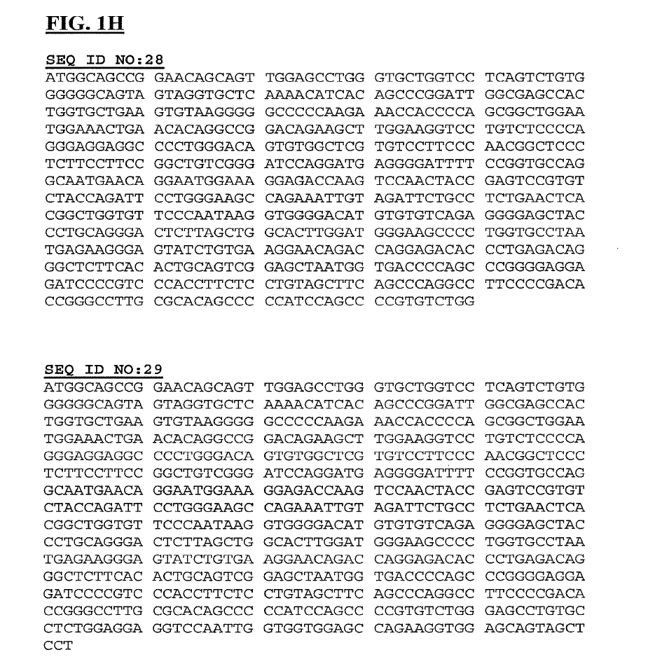

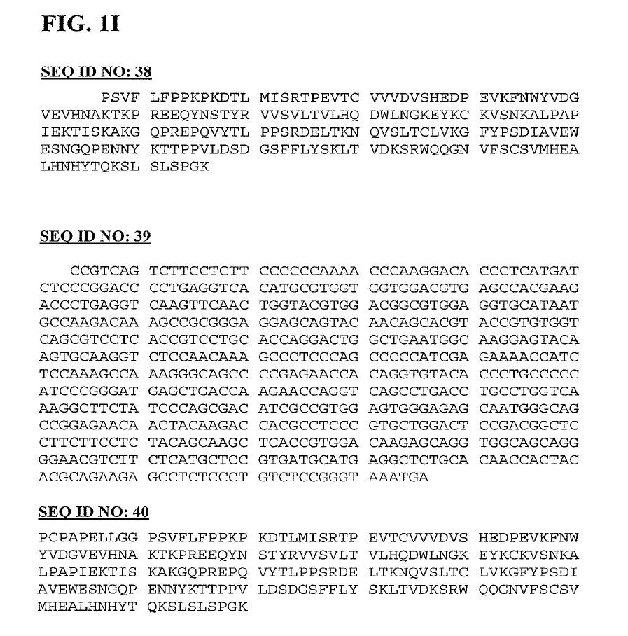

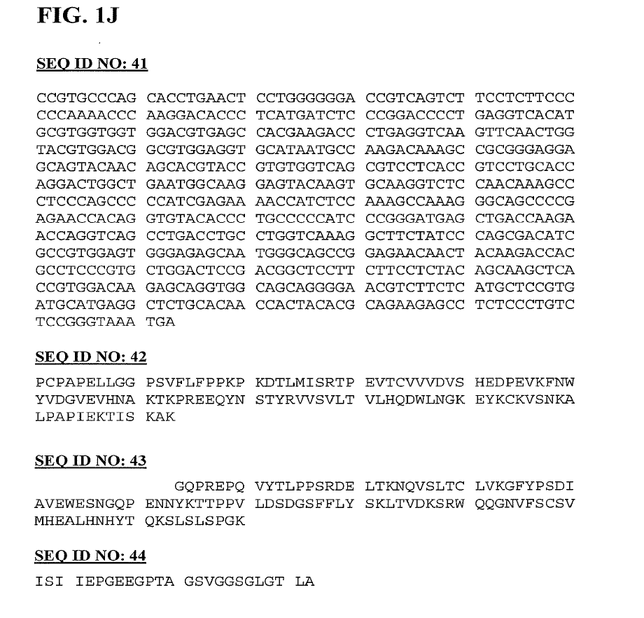

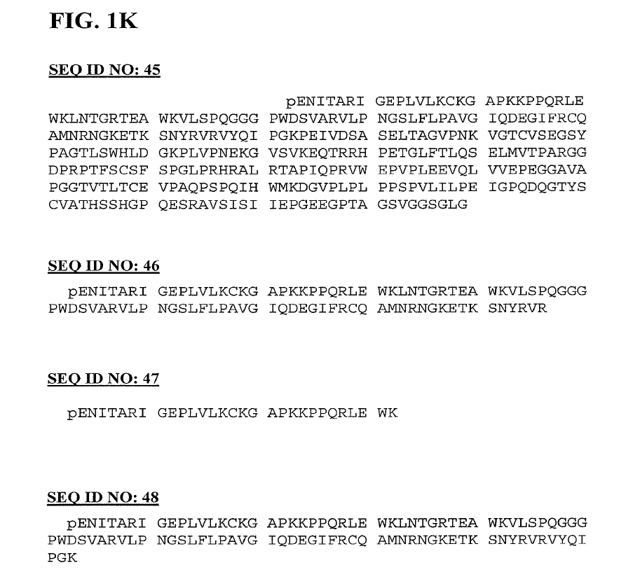

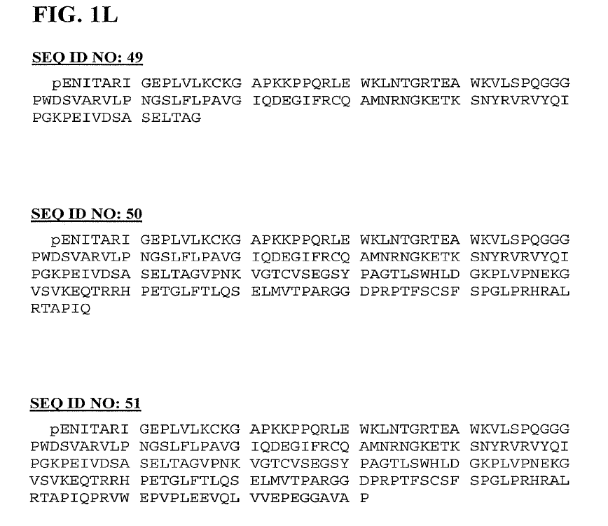

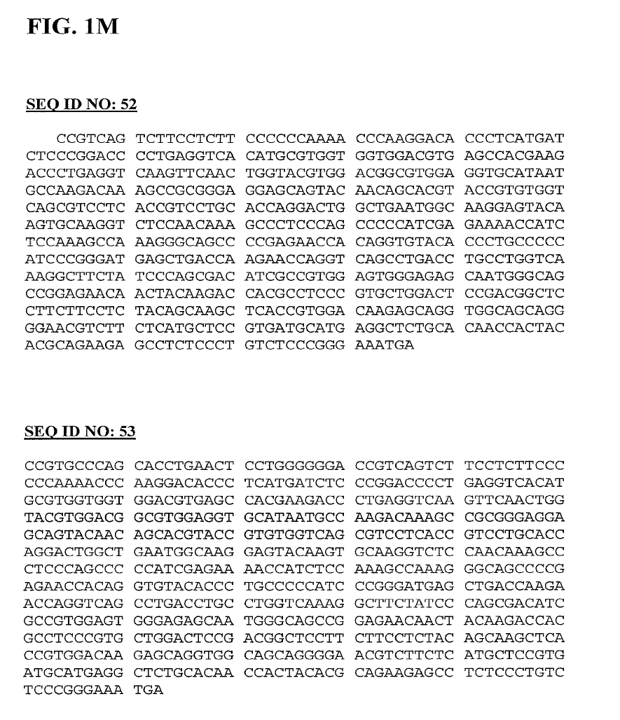

FIGS. 1-8 provide examples of various amino acid and nucleic acid constructs of the present invention.

For example, FIG. 1 provides the amino acid sequences for full length human RAGE (SEQ ID NO: 1), full-length human RAGE with signal sequences corresponding to amino acids 1-22 removed (SEQ ID NO: 2) and 1-23 removed (SEQ ID NO: 3) (FIG. 1A). Also provided in FIG. 1 are various fragments of human RAGE that may be used in the RAGE fusion proteins of the present invention (SEQ ID NOs: 4-24 and 44) (FIG. 1C-1F, and FIG. 1J), as well as DNA sequences that encode for these RAGE polypeptide sequences and fragments of RAGE polypeptide sequences (SEQ ID NOs: 25-29) (FIG. 1G-1H). Also shown in FIG. 1 (FIG. 1I and FIG. 1J) are amino acid sequences for a human immunoglobulin polypeptide comprising a C_{H}2 and a C_{H}3 domain, wherein the C_{H}2 does not include at least part of the hinge region (SEQ ID NO: 38), an amino acid sequence for a human immunoglobulin polypeptide comprising a C_{H}2 and a C_{H}3 domain, wherein the C_{H}2 does include at least part of the hinge region (SEQ ID NO: 40), an amino acid sequence for a human immunoglobulin polypeptide comprising a C_{H}2 domain including at least part of the hinge (SEQ ID NO: 42), an amino acid sequence for a human immunoglobulin polypeptide comprising a C_{H}3 domain, as well as DNA sequences encoding a human immunoglobulin polypeptide comprising a C_{H}2 and a C_{H}3 domain, where the C_{H}2 does not include at least part of the hinge region (SEQ ID NO: 41) and where the C_{H}2 does include at least part of the hinge region (SEQ ID NO: 39). Also provided in FIG. 1 are sequences for fragments of RAGE wherein the N-terminal glutamine (Q) is cyclized to pyroglutamic acid (FIGS. 1K and 1L). FIG. 1M provides SEQ ID NOs: 52 and 53 which are alternate nucleic acid sequences that encode the immunoglobulin polypeptides of SEQ ID NOs: 38 and 40, respectively.

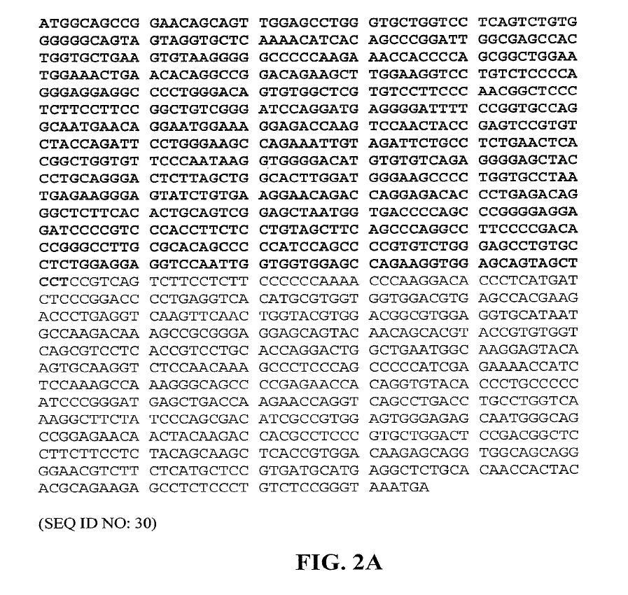

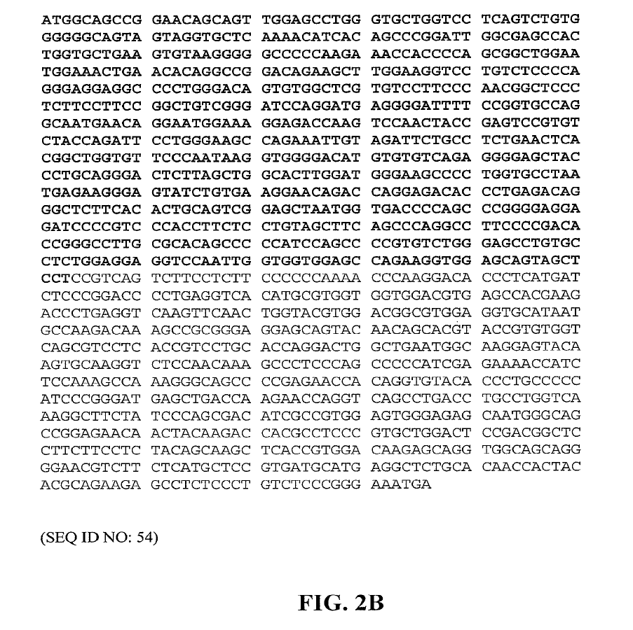

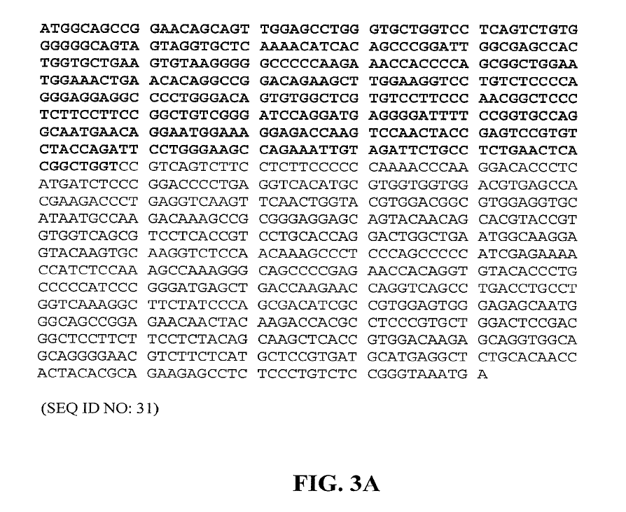

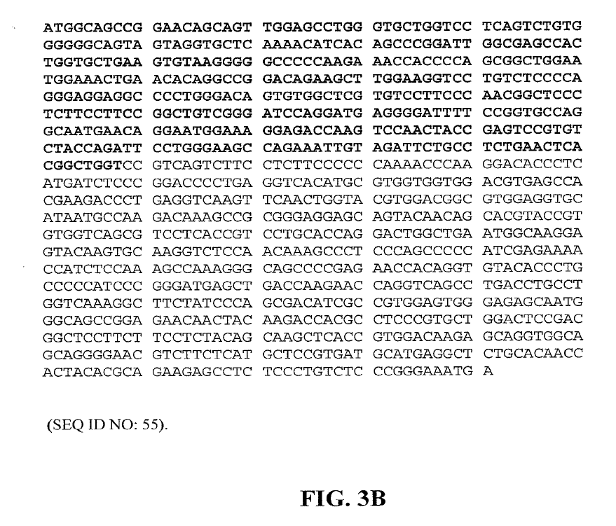

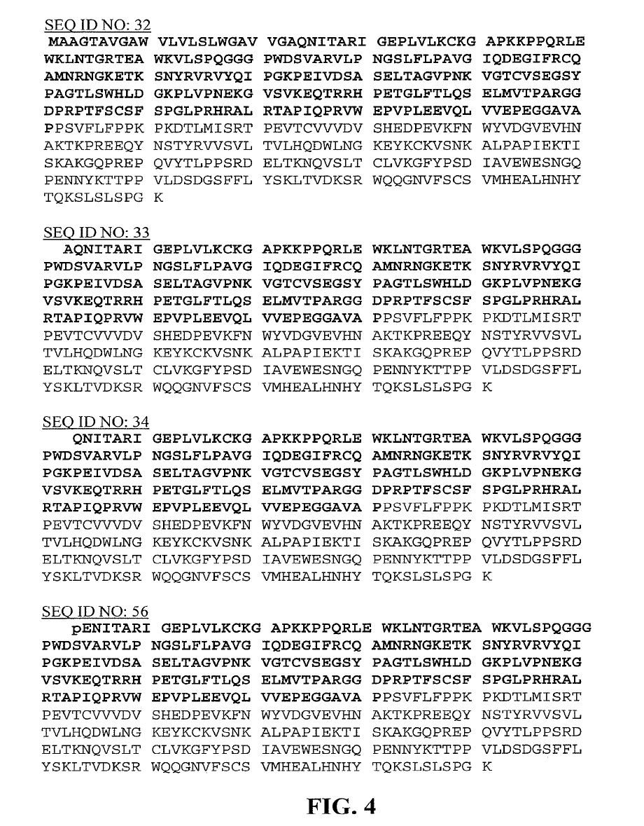

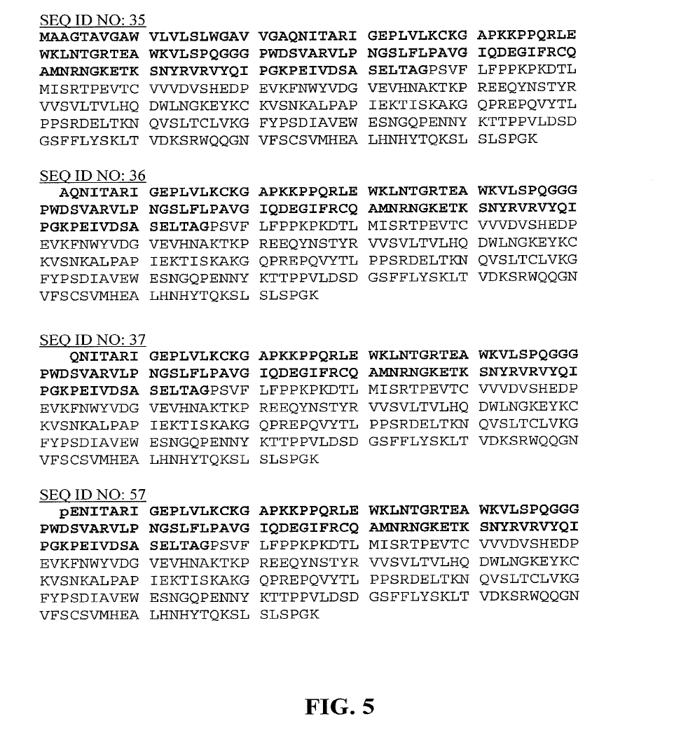

As described in more detail below, FIGS. 2 and 3 provide the sequences of various nucleic acid constructs that encode for RAGE fusion proteins of the present invention. FIGS. 4 and 5 provide amino acid sequences for various RAGE fusion proteins of the present invention.

The location of mutant residues is delineated herein with respect to mammalian RAGE having the N-terminal sequence, corresponding to amino acids 1-23 of the full length protein, removed. For example, N2 corresponds to a glutamine residue (N) at position 2 of the RAGE polypeptide without the signal sequence (SEQ ID NO: 3); this would correspond to glutamine at position 25 of the full-length RAGE (SEQ ID NO: 1) (see FIG. 1). Thus, the fusion protein of any of the previous embodiments may comprise a RAGE polypeptide comprising a fragment of the human RAGE peptide of SEQ ID NO: 3. Thus, as noted above, in the description of specific point mutations used herein, the location of the RAGE and/or immunoglobulin polypeptide mutations is based on residue numbers of the amino acid sequence of SEQ ID NO: 3 or a RAGE fusion protein having two RAGE domains and as shown in SEQ ID NO: 34. The amino acid sequence of SEQ ID NO: 3 corresponds to the full-length sequence of human RAGE without the signal sequence (i.e., SEQ ID NO: 1 without residues 1-23). The amino acid sequence of SEQ ID NO: 34 corresponds to the sequence of a human RAGE fusion protein having two RAGE domains without the signal sequence (i.e., SEQ ID NO: 1 without residues 1-23). It will be understood by those in the art that the mutations described for a fusion protein having two RAGE domains (e.g., SEQ ID NO: 34) may be made in analogous positions in a RAGE fusion protein having only one RAGE domain or in RAGE fusion proteins having three RAGE domains. This furin cleavage site is not present in the shorter RAGE fusion proteins having only one domain as illustrated in SEQ ID NOs: 57, 37, 36, and 35.

In certain embodiments of the fusion protein where the RAGE polypeptide comprises a point mutation to remove a glycosylation site, the point mutation may be in the RAGE ligand binding domain. In such an embodiment, the point mutation to remove a glycosylation site may be, alternatively, within the first 100, 90, 80, 70, 60, 50, 40, or 30 amino acids of the N-terminus of the fusion protein that does not include the signal sequence.

In another embodiment, the present invention provides a fusion protein of any of the previous embodiments wherein: (a) the RAGE polypeptide comprises a fragment of human wild type RAGE peptide (SEQ ID NO:3) comprising a RAGE ligand binding domain and at least the first 200 amino acids of SEQ ID NO:3, and wherein the RAGE polypeptide comprises: (i) a point mutation or mutations at one or both of residues 2 or 58 to remove and/or alter a glycosylation site; and/or (ii) a point mutation at one or more of residues 193 to 198 to remove and/or alter a furin cleavage site; and (b) the immunoglobulin polypeptide comprises at least a fragment of a C_{H}2 domain and a C_{H}3 domain of an immunoglobulin. In some embodiments, the present invention provides a fusion protein of any of the previous embodiments wherein the RAGE ligand binding domain comprises the most N-terminal domain of the fusion protein.

In some embodiments, the RAGE polypeptide comprises a point mutation selected from the group consisting of: (a) N2Q; (b) N58Q; (c) N2Q and N58Q, wherein the position of the mutation is relative to SEQ ID NO: 3, and wherein RAGE polypeptide comprises SEQ ID NO: 13, 14, 15, 16, 46, 48, or 49. In other embodiments, the RAGE polypeptide comprises a point mutation selected from the group consisting of: (a) N2Q; (b) N58Q; (c) N2Q and N58Q, wherein the position of the mutation is relative to SEQ ID NO: 3, and wherein RAGE polypeptide is SEQ ID NO: 13, 14, 15, 16, 46, 48, or 49.

In yet other embodiments, the point mutation corresponds to a point mutation selected from the group consisting of: (a) R195A; (b) R195K; (c) R198A; (d) R198K; (e) R198H; and (f) R198T, wherein the position of the mutation is relative to SEQ ID NO: 3, and wherein RAGE polypeptide comprises SEQ ID NO: 5, 6, 17, 18, 19, 20, 45, 50, or 51. Or, the point mutation corresponds to a point mutation selected from the group consisting of: (a) R195A; (b) R195K; (c) R198A; (d) R198K; (e) R198H; and (f) R198T, wherein the position of the mutation is relative to SEQ ID NO: 3, and wherein RAGE polypeptide is SEQ ID NO: 5, 6, 17, 18, 19, 20, 45, 50, or 51.

In yet other embodiments, the point mutation corresponds to a point mutation selected from the group consisting of: (a) N2Q; (b) N58Q; (c) N2Q and N58Q; (d) R195A; (e) R195K; (f) R198A; (g) R198K; (h) R198H; (i) R198T; (j) N2Q and R198A; (k) N58Q and R198A; and (l) N2Q, N58Q, and R198A wherein the position of the mutation is relative to SEQ ID NO: 3, and wherein RAGE polypeptide comprises SEQ ID NO: 5, 6, 17, 18, 19, 20, 45, 50, or 51. In yet other embodiments, the point mutation corresponds to a point mutation selected from the group consisting of: (a) N2Q; (b) N58Q; (c) N2Q and N58Q; (d) R195A; (e) R195K; (f) R198A; (g) R198K; (h) R198H; (i) R198T; (j) N2Q and R198A; (k) N58Q and R198A; and (l) N2Q, N58Q, and R198A wherein the position of the mutation is relative to SEQ ID NO: 3, and wherein RAGE polypeptide is SEQ ID NO: 5, 6, 17, 18, 19, 20, 45, 50, or 51.

In yet other embodiments, the point mutation corresponds to a point mutation selected from the group consisting of R195H, R195T, and/or G59S either alone or in any possible combination, and/or the mutations described above in combination with at least one of R195H, R195T, and/or G59S, wherein the position of the mutation is relative to SEQ ID NO: 3, and wherein RAGE polypeptide comprises SEQ ID NO: 5, 6, 17, 18, 19, 20, 45, 50, or 51. In yet other embodiments, the point mutation corresponds to a point mutation selected from the group consisting of R195H, R195T, and/or G59S either alone or in any possible combination, and/or mutations described above in wherein the position of the mutation is relative to SEQ ID NO: 3, and wherein RAGE polypeptide is SEQ ID NO: 5, 6, 17, 18, 19, 20, 45, 50, or 51.

Table 1 below provides a non-limiting compilation of example mutations of the present invention where the position of the mutation is provided relative to the RAGE fusion protein having the amino acid sequence as set forth in SEQ ID NO: 34. The actual amino acid sequences of Table 1 (i.e., SEQ ID NOs: 62 to 81, SEQ ID NOs: 92 to 190 and SEQ ID NOs: 58-61, 82, 200 and 201) are shown in FIG. 6A-6EE.

TABLE 1

| | Processing Variant of TTP4000 (SEQ ID NO:) | | | | | |
|---|---|---|---|---|---|---|
| | | | | | | (−) 1-23 |
| | | (−) 1-23 | | | (−) 1-24 | Signal |
| | | Signal | | | Signal | (−) C- |
| | | (−) C- | (−) 1-23 | | (−) C- | terminal |
| | (−) 1-23 | terminal | Signal | (−) 1-24 | terminal | K |
| Mutation | Signal | K | (+) pE | Signal | K | (+) pE |
| N2Q | 62 | 92 | 112 | 132 | 152 | 172 |
| N58Q | 63 | 93 | 113 | 133 | 153 | 173 |
| N2Q, N58Q | 64 | 94 | 114 | 134 | 154 | 174 |
| R195A | 65 | 95 | 115 | 135 | 155 | 175 |
| R195K | 66 | 96 | 116 | 136 | 156 | 176 |
| R198A | 67 | 97 | 117 | 137 | 157 | 177 |
| R198K | 68 | 98 | 118 | 138 | 158 | 178 |
| R198H | 69 | 99 | 119 | 139 | 159 | 179 |
| R198T | 70 | 100 | 120 | 140 | 160 | 180 |
| N2Q, R198A | 71 | 101 | 121 | 141 | 161 | 181 |
| N58Q, R198A | 72 | 102 | 122 | 142 | 162 | 182 |
| N2Q, N58Q, | 73 | 103 | 123 | 143 | 163 | 183 |
| R198A | | | | | | |
| N288Q | 74 | 104 | 124 | 144 | 164 | 184 |
| N2Q, N288Q | 75 | 105 | 125 | 145 | 165 | 185 |
| N58Q, N288Q | 76 | 106 | 126 | 146 | 166 | 186 |
| N2Q, N58Q, | 77 | 107 | 127 | 147 | 167 | 187 |
| N288Q | | | | | | |
| N2Q, R198A, | 78 | 108 | 128 | 148 | 168 | 188 |
| N288Q | | | | | | |
| N58Q, R198A, | 79 | 109 | 129 | 149 | 169 | 189 |
| N288Q | | | | | | |
| N2Q, N58Q, | 80 | 110 | 130 | 150 | 170 | 190 |
| R198A, | | | | | | |
| N288Q | | | | | | |
| G59S, R198A | 81 | 111 | 131 | 151 | 171 | 200 |
| N2Q, G59S, | 58 | 59 | 60 | 61 | 82 | 201 |
| R198A | | | | | | |

In other embodiments, the fusion protein of the present invention comprises an amino acid sequence at least 70%, 75%, 80%, 85%, 90%, 95%, 96%, 97%, 98%, or 99% identical to one of the sequences described above. In other embodiments, the fusion protein of the present invention comprises an amino acid sequence at least 70%, 75%, 80%, 85%, 90%, 95%, 96%, 97%, 98%, or 99% identical to SEQ ID NOs: 62 to 81, SEQ ID NOs: 92 to 190, or SEQ ID NOs: 58-61, 82, 200 or 201 wherein the RAGE polypeptide portion of the RAGE fusion protein comprises: (i) a point mutation or mutations at one or more of residues to remove and/or alter a glycosylation site; and/or (ii) a point mutation at one or more of residues to remove and/or alter a furin cleavage site.

In another embodiment, the fusion protein of the present invention comprises an amino acid sequence as set forth in any one of SEQ ID NOs: 62 to 81, SEQ ID NOs: 92 to 190 or SEQ ID NOs: 58-61, 82, 200 or 201. In another embodiment, the fusion protein of the present invention is the amino acid sequence of any one of SEQ ID NOs: 62 to 81, SEQ ID NOs: 92 to 190, or SEQ ID NOs: 58-61, 82, 200 or 201.

A RAGE polypeptide of the fusion proteins of the invention may comprise a fragment of a mammalian wild type RAGE peptide comprising a RAGE ligand binding domain. In an embodiment, a fragment of a RAGE peptide may comprise at least an uninterrupted 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 amino acid sequence from the wild-type RAGE peptide. In an embodiment, the RAGE polypeptide comprises a fragment of the human wild type RAGE peptide of SEQ ID NO: 6 (FIG. 1C).

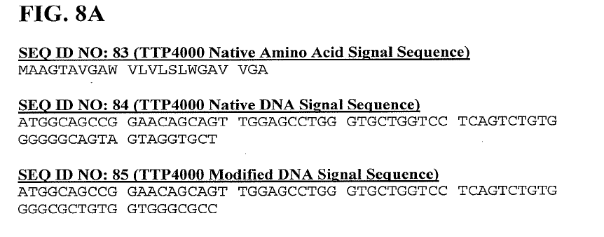

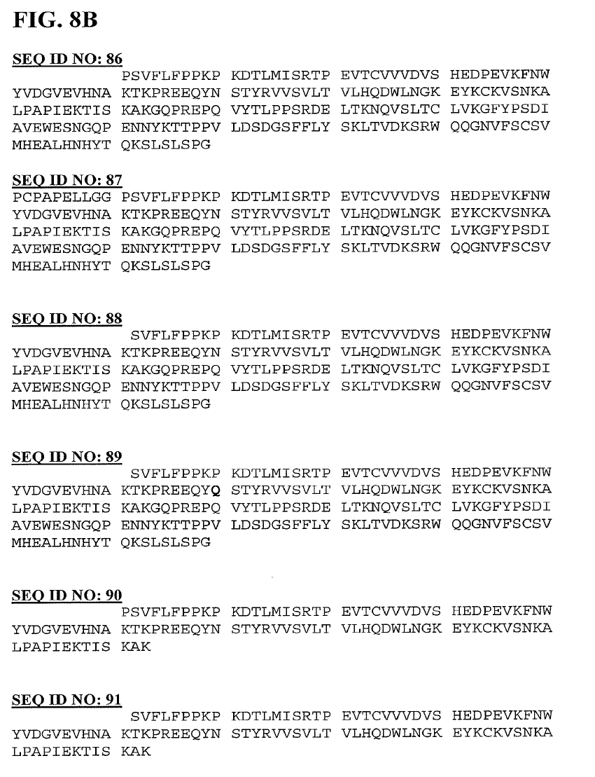

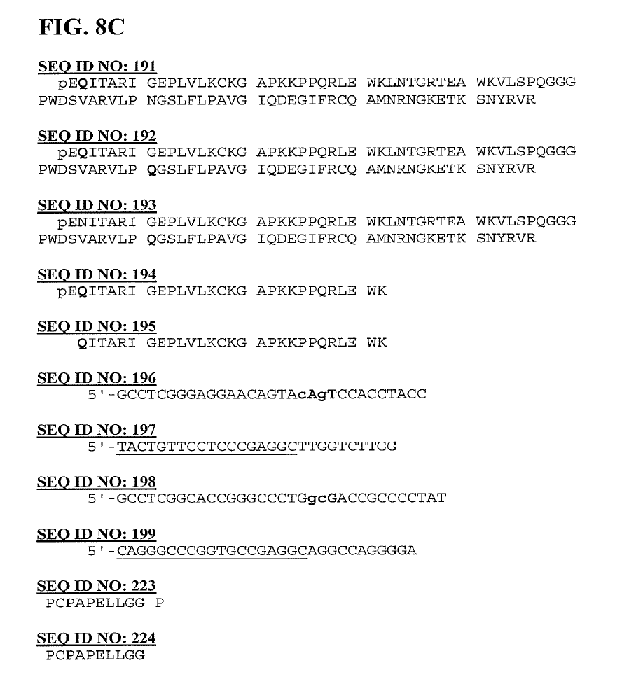

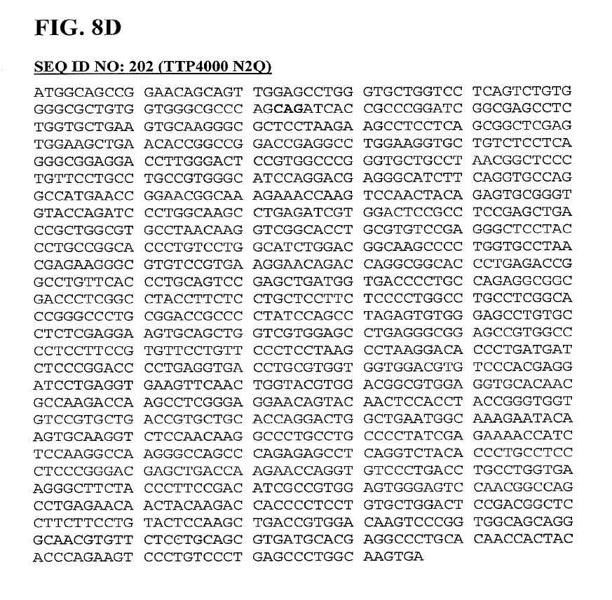

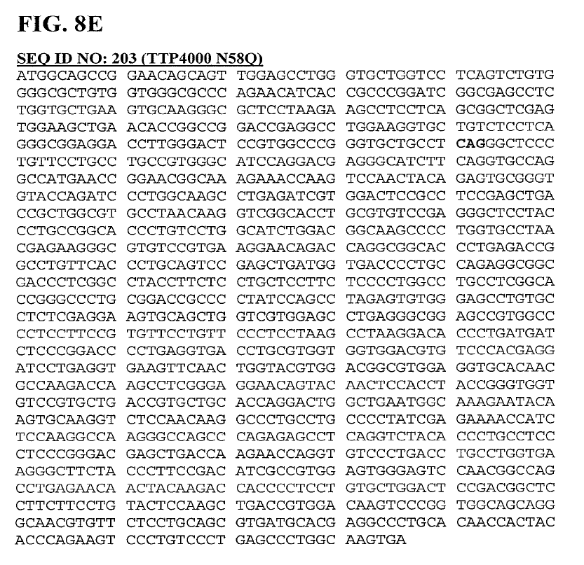

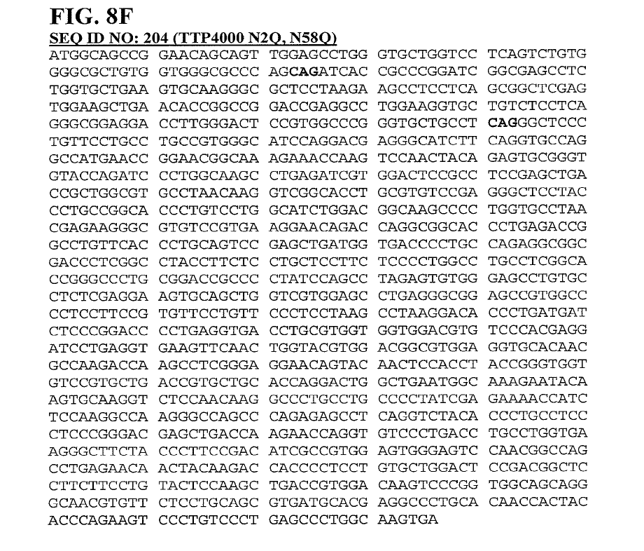

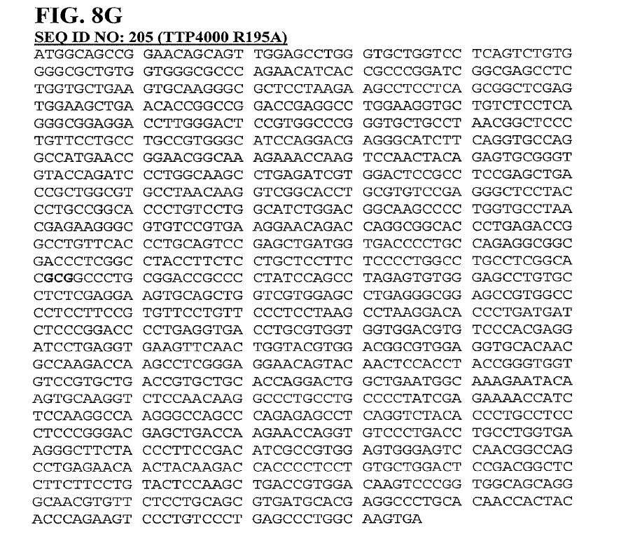

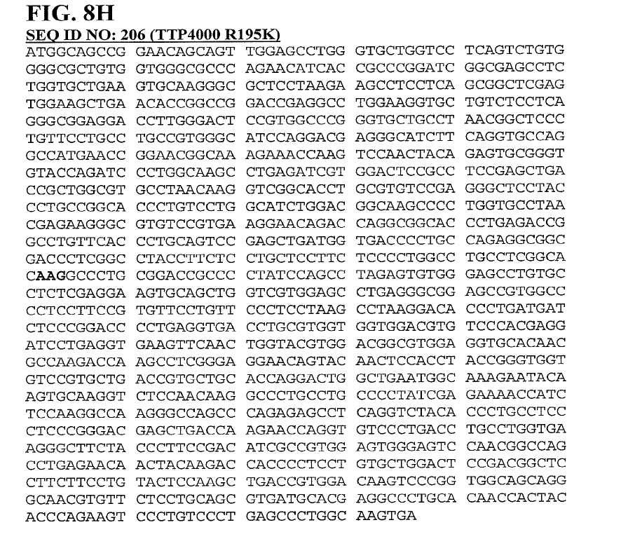

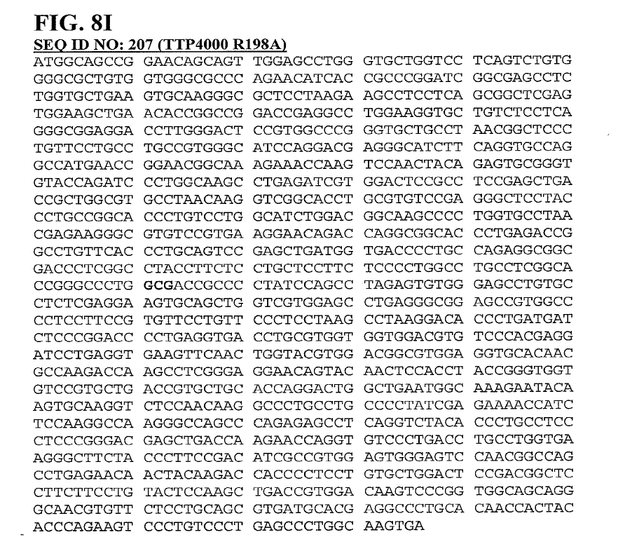

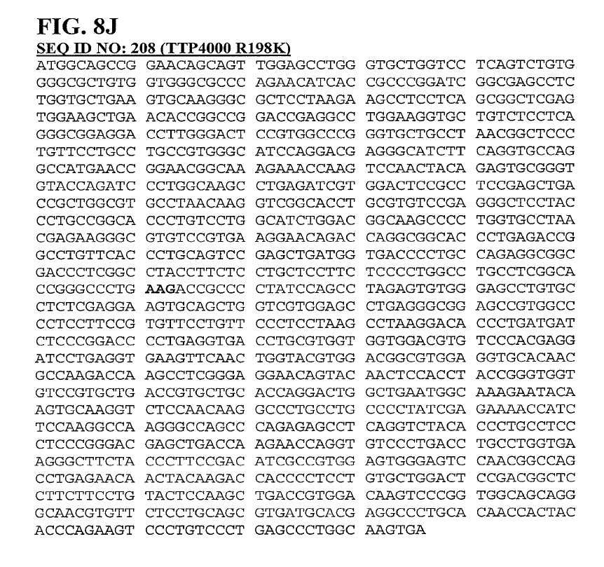

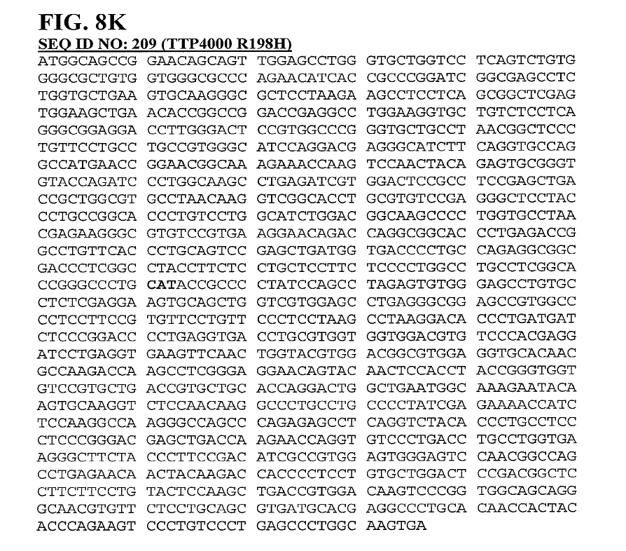

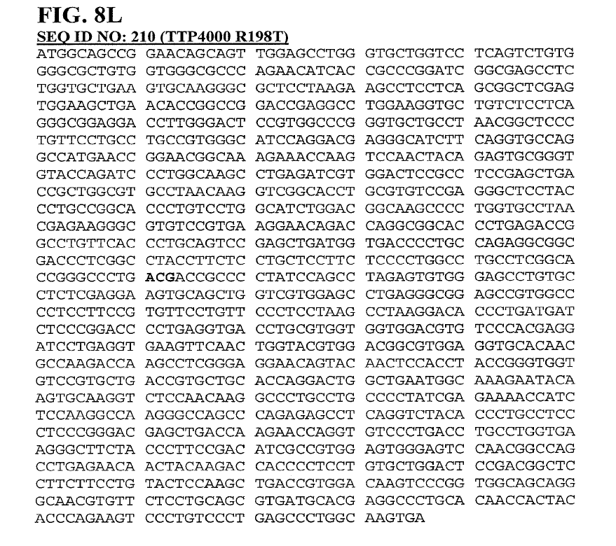

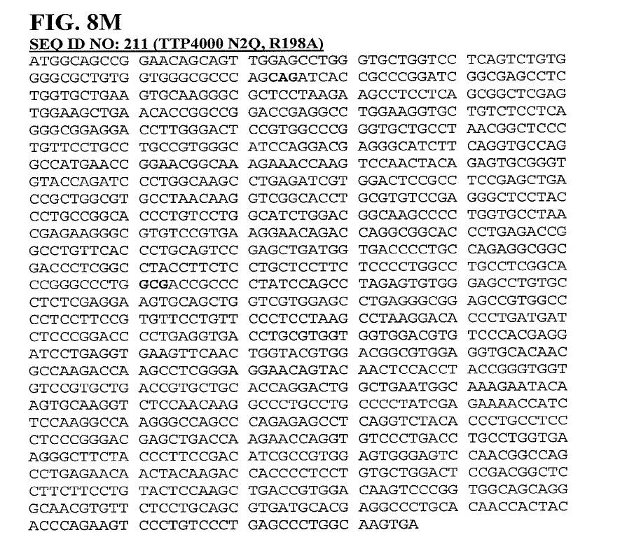

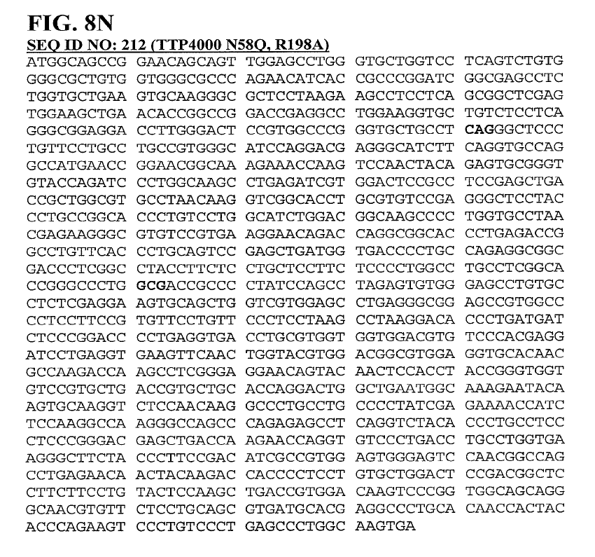

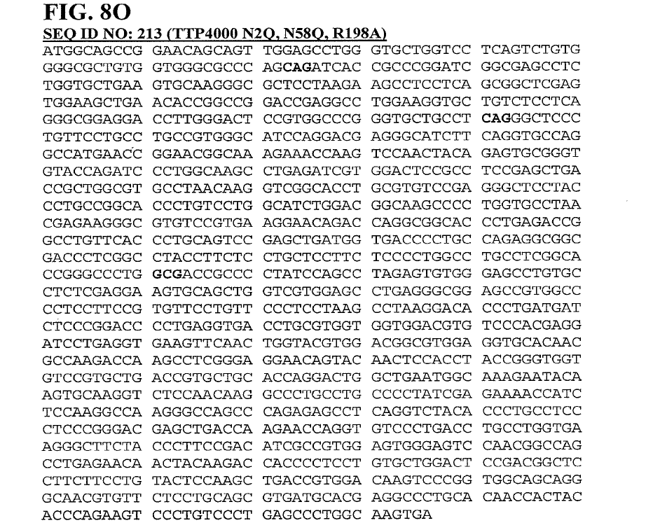

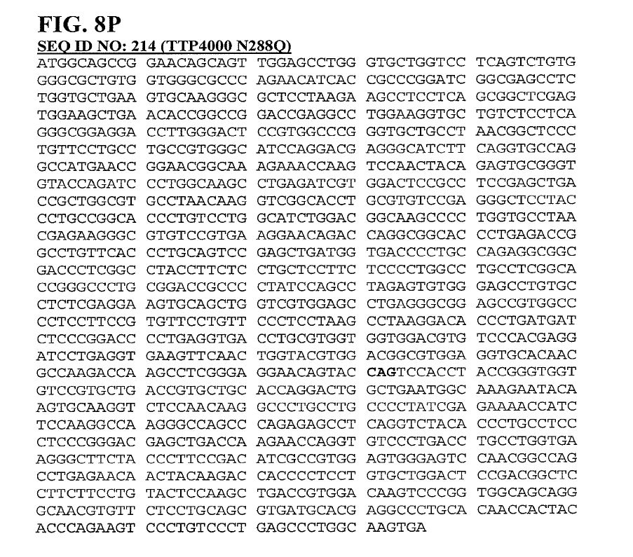

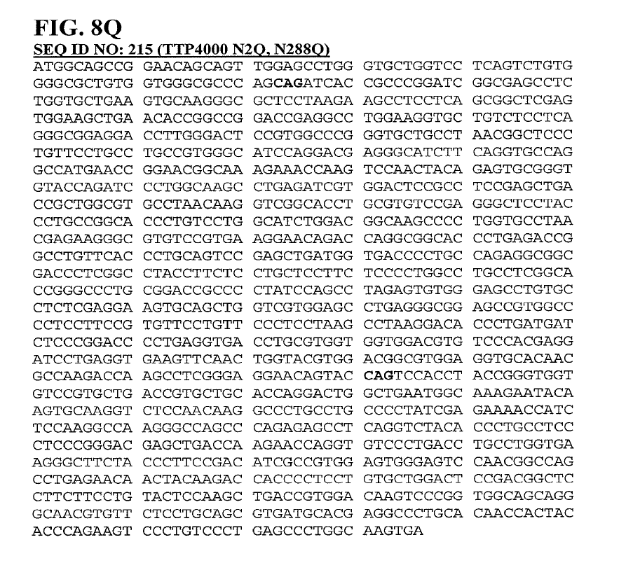

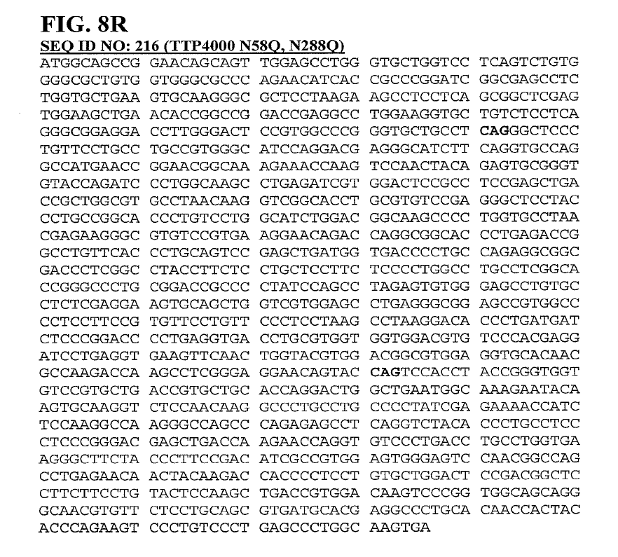

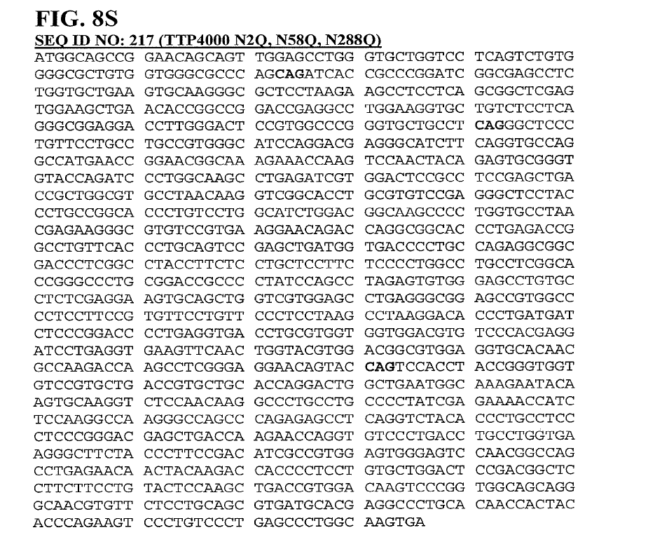

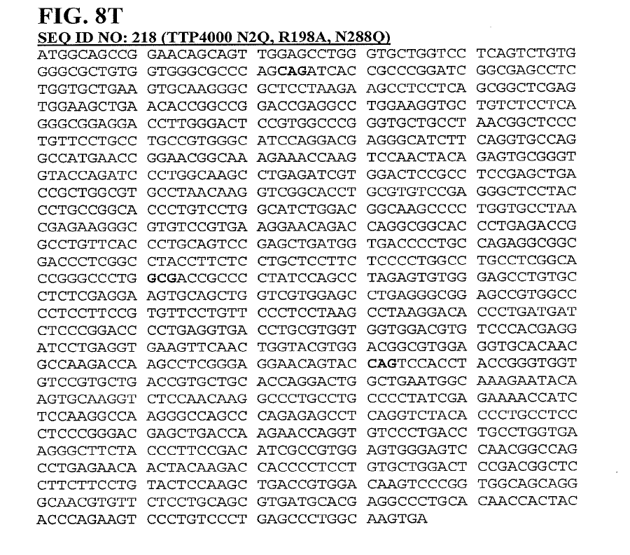

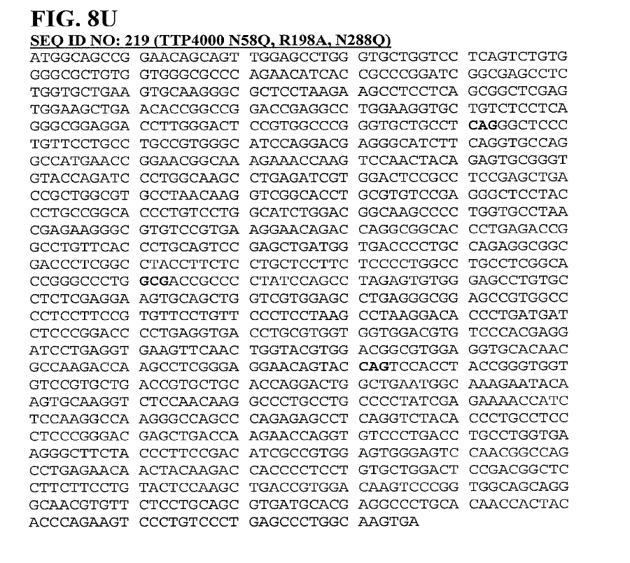

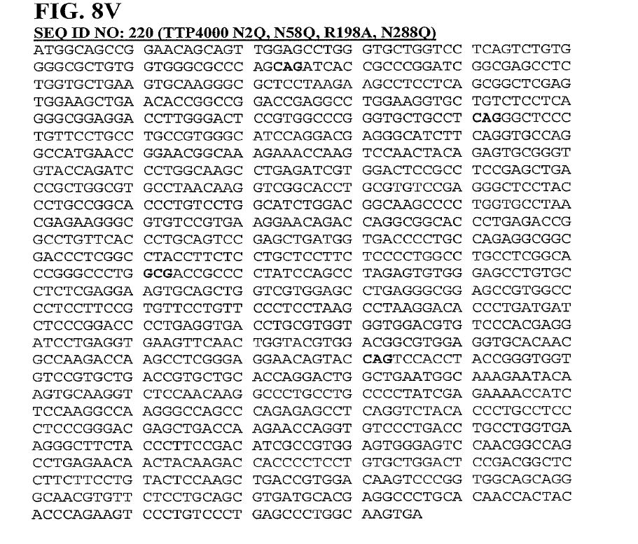

The RAGE ligand binding domain of the RAGE polypeptide of any of the previous embodiments may comprise a RAGE V domain, see e.g., SEQ ID NO: 7, SEQ ID NO: 8, SEQ ID NO: 46 (FIGS. 1D and 1K) and SEQ ID NO: 191, SEQ ID NO: 192, or SEQ ID NO: 193 in FIG. 8C). Or, a sequence at least 95% identical to the RAGE V domain or a fragment thereof may be used.

In another embodiment, the RAGE ligand binding domain of any of the previous embodiments may comprise SEQ ID NO: 9, or a sequence at least 95% identical thereto, or SEQ ID NO: 10, or a sequence at least 95% identical thereto (FIG. 1D), or SEQ ID NO: 47, or a sequence at least 95% identical thereto (FIG. 1K), or SEQ ID NO: 194, or a sequence at least 95% identical thereto, or SEQ ID NO: 195, or a sequence at least 95% identical thereto (FIG. 8C).

In another embodiment, the ligand binding domain may comprise amino acids 23-53 of SEQ ID NO. 1 (FIG. 1). In another embodiment, the ligand binding domain may comprise amino acids 24-52 of SEQ ID NO: 1. In another embodiment, the ligand binding domain may comprise amino acids 31-52 of SEQ ID NO: 1. In another embodiment, the ligand binding domain may comprise amino acids 31-116 of SEQ ID NO: 1. In another embodiment, the ligand binding domain may comprise amino acids 19-52 of SEQ ID NO: 1. For example, the ligand binding domain may comprise, a RAGE V domain or a portion thereof such as the RAGE ligand binding domain (e.g., amino acids 1-118, 23-118, 24-118, 31-118, 1-116, 23-116, 24-116, 31-116, 1-54, 23-54, 24-54, 31-54, 1-53, 23-53, 24-53, or 31-53 of SEQ ID NO: 1, or fragments thereof). Or fragments of the polypeptides that functionally bind a RAGE ligand may be used. Or, a sequence at least 70%, 75%, 80%, 85%, 90%, 95%, 97%, 98% or 99% identical to the RAGE V domain or a fragment thereof (e.g., as described above) may be used.

Further, as is known in the art, in embodiments where the N-terminus of the fusion protein is glutamine, as for example upon removal of the signal sequence comprising residues 1-23 of SEQ ID NO: 1, the glutamine (e.g., Q24 for a polypeptide comprise amino acids 24-118 or SEQ ID NO: 1) may cyclize to form pyroglutamic acid (pE). As described herein, in each of these embodiments having an N-terminal glutamine cyclize to pE, the sequences comprising a RAGE ligand binding domain may contain at least one point mutation to alter a glycosylation site, or contain one point mutation to remove a glycosylation site, or may contain two or more point mutations to remove and/or alter two glycosylation sites. Also, the RAGE sequence may comprise a mutation to alter and/or remove an enzyme (e.g., furin) cleavage site.

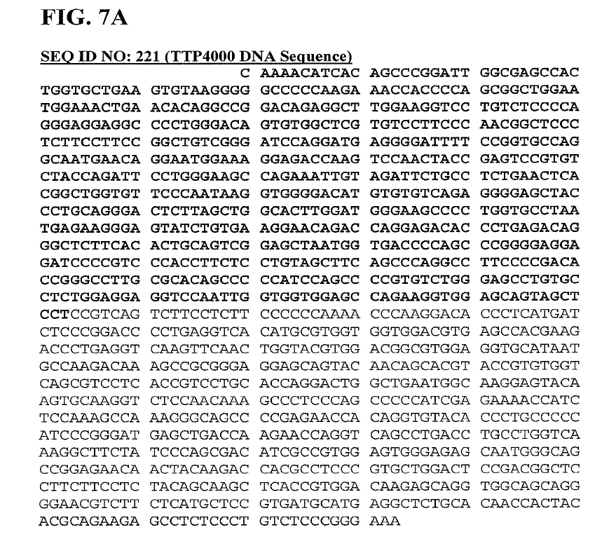

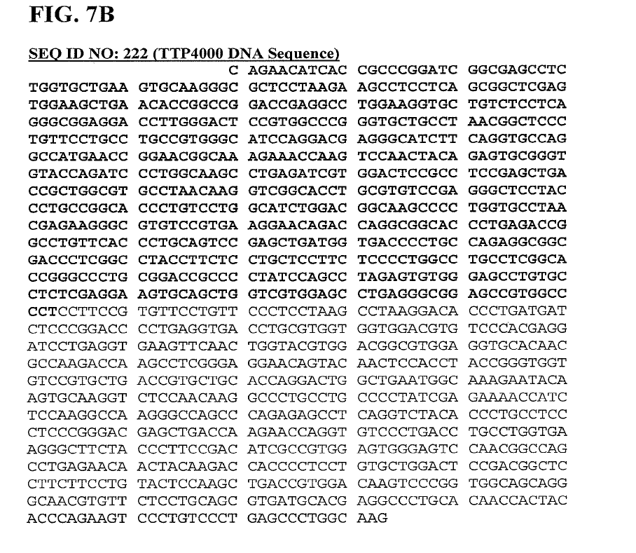

As described in more detail herein, the RAGE fusion proteins of the present invention may be expressed in cells. FIG. 7 provides nucleic acid sequences that may be used to encode a RAGE fusion protein of the present invention, wherein SEQ ID NO: 221 comprises a wild-type nucleic acid sequence (i.e., wild type for human RAGE and human immunoglobulin) and a nucleic acid sequence (SEQ ID NO: 222) optimized for expression. Also, FIG. 8A provides wild-type and modified nucleic acid sequences (SEQ ID NOs: 84 and 85) corresponding to the signal sequence of human RAGE.

Thus, in certain embodiments, the RAGE polypeptide may further comprise signal sequence residues. For example, the signal sequence of a fusion protein may comprise the native human RAGE signal sequence (SEQ ID NO: 83) or signal sequence that has been modified from the native sequence (e.g., SEQ ID NO: 84), or a signal sequence where the leader sequence has a glycine as a first residue rather than methionine (see e.g., Neeper et al., 1992).

As recognized in the art, the C_{H}3 region of the RAGE fusion proteins of the present invention may have the C-terminal amino acid cleaved off through a post-translational modification when expressed in certain recombinant systems. (See e.g., Li, et al., BioProcessing J., 4:23-30 (2005)). In an embodiment, the C-terminal amino acid cleaved off is lysine (K). Thus, in alternate embodiments of each of the proteins and protein compositions of the invention, the RAGE fusion protein of the present invention may comprise a polypeptide having the amino acid sequence without the C-terminal lysine (K). Non-limiting examples of such fusion proteins include SEQ ID NOs: 92-111, 152-171, 59 and 82.

As discussed herein, the fusion protein may also comprise a polypeptide derived from an immunoglobulin. In various embodiments, and as illustrated in the example embodiments disclosed herein, the RAGE fusion protein comprises a point mutation at residue to remove a glycosylation site within the immunoglobulin polypeptide of the RAGE fusion protein. For example, in one embodiment, the RAGE fusion protein may comprise a mutation at position 288 (e.g., N288Q) relative to the sequence as set forth in SEQ ID NO: 34 and/or position 173 (e.g., N173Q) relative to the sequence as set forth in SEQ ID NO: 37.

In certain embodiments, the immunoglobulin polypeptide comprises a C_{H}2 domain or a fragment of a C_{H}2 domain that does not include at least a portion of the hinge region. For example, in alternate embodiments, the hinge region that is not included as part of the C_{H}2 domain has the sequence as set forth in SEQ ID NOs: 223 or 224. In certain embodiments, the C_{H}2 domain, or fragment thereof, is linked via its C-terminus to the N-terminus of a C_{H}3 domain, or fragment thereof, of an immunoglobulin polypeptide.

Thus, in certain embodiments, the immunoglobulin polypeptide comprises at least a fragment of a C_{H}2 domain and at least a fragment of a C_{H}3 domain of an immunoglobulin. In one embodiment, the immunoglobulin polypeptide may comprise an immunoglobulin heavy chain or a portion (i.e., fragment) thereof. For example, the heavy chain fragment may comprise a polypeptide derived from the Fc fragment of an immunoglobulin, wherein the Fc fragment comprises the heavy chain hinge polypeptide, and C_{H}2 and C_{H}3 domains of the immunoglobulin heavy chain as a monomer. The heavy chain (or portion thereof) may be derived from any one of the known heavy chain isotypes: IgG (γ), IgM (μ), IgD (δ), IgE (ε), or IgA (α). In addition, the heavy chain (or portion thereof) may be derived from any one of the known heavy chain subtypes: IgG1 (γ1), IgG2 (γ2), IgG3 (γ3), IgG4 (γ4), IgA1 (α1), IgA2 (α2), or mutations of these isotypes or subtypes that alter the biological activity. The immunoglobulin may comprise the C_{H}2 and C_{H}3 domains of a human IgG1 or portions of either, or both, of these domains. As an example embodiment, the polypeptide comprising the C_{H}2 and C_{H}3 domains of a human IgG1 or a portion thereof may comprise SEQ ID NO: 40 (FIG. 1) or a portion thereof. In one embodiment, the polypeptide comprising the C_{H}2 and C_{H}3 domains of a human IgG1, or a portion thereof, may comprise SEQ ID NO: 38 or a fragment thereof. The immunoglobulin peptide may be encoded by the nucleic acid sequence of SEQ ID NO: 39 or SEQ ID NO: 41 (FIG. 1). The immunoglobulin sequence in SEQ ID NO: 38 or SEQ ID NO: 40 may also be encoded by SEQ ID NO: 52 or SEQ ID NO: 53 (FIG. 1), where silent base changes for the codons that encode for proline (CCG to CCC) and glycine (GGT to GGG) at the C-terminus of the sequence remove a cryptic RNA splice site near the terminal codon (i.e., nucleotides 622-627 of SEQ ID NO: 39 are modified to generate SEQ ID NO: 52 or nucleotides 652-657 of SEQ ID NO: 41 are modified to generate SEQ ID NO: 53). In another embodiment, the C_{H}2 domain, or a fragment thereof comprises SEQ ID NO: 42 (FIG. 1). In another embodiment, the fragment of SEQ ID NO: 42 comprises SEQ ID NO: 42 with the first ten or eleven amino acids comprising at least a portion of the Fc hinge region removed (see e.g., SEQ ID NO: 90 or 91).

The hinge region of the Fc portion of the immunoglobulin chain may be proinflammatory in vivo. Thus, in one embodiment, the RAGE fusion protein of the present invention comprises an interdomain linker derived from RAGE rather than an interdomain hinge polypeptide derived from an immunoglobulin.

Thus in certain embodiments, the RAGE fusion protein may comprise a RAGE polypeptide directly linked to an immunoglobulin polypeptide, wherein the immunoglobulin polypeptide comprises at least a fragment of a C_{H}2 domain and at least a fragment of a C_{H}3 domain of an immunoglobulin. In one embodiment, the C_{H}2 domain, or a fragment thereof, comprises SEQ ID NO: 42 (FIG. 1). In an embodiment, the fragment of SEQ ID NO: 42 comprises SEQ ID NO: 42 with the first ten or eleven amino acids comprising at least a portion of the Fc hinge region removed (See SEQ ID NO: 90 or 91) (FIG. 8B). For example, as noted above, in certain embodiments, this 11 or 10 amino acid hinge region that has been removed from the C_{H}2 domain has the sequence as set forth in SEQ ID NOs: 223 or 224, respectively (FIG. 8C).

As noted above, in certain embodiments, the C_{H}2 domain is linked via its C-terminus to the N-terminus of a C_{H}3 domain of an immunoglobulin polypeptide. For example, in certain embodiments, the immunoglobulin polypeptide of any one of the previous embodiments of the fusion protein may comprise any one of SEQ ID NOS: 86, 87, 88, or 89. As shown in FIG. 8B, SEQ ID NO: 89 comprises a mutation corresponding to N288Q as set forth in the RAGE fusion protein of SEQ ID NO: 34 so as to remove a glycosylation site in the immunoglobulin portion of the RAGE fusion protein.

The RAGE polypeptide used in the RAGE fusion proteins of the present invention may comprise a RAGE immunoglobulin domain or multiple RAGE immunoglobulin domains. Additionally or alternatively, the fragment of RAGE may comprise an interdomain linker. For example, the RAGE polypeptide may comprise a RAGE immunoglobulin domain linked to an upstream (i.e., closer to the N-terminus) or downstream (i.e., closer to the C-terminus) interdomain linker.

In some embodiments, the RAGE polypeptide may comprise two (or more) RAGE immunoglobulin domains each linked to each other by an interdomain linker. In such embodiments, the RAGE polypeptide may comprise multiple RAGE immunoglobulin domains linked to each other by one or more interdomain linkers. The RAGE polypeptide may further comprise a terminal interdomain linker attached to the N-terminal RAGE immunoglobulin domain and/or the C-terminal immunoglobulin domain. Additional combinations of RAGE immunoglobulin domains and interdomain linkers are within the scope of the present invention.

In one embodiment, the RAGE polypeptide comprises a RAGE interdomain linker linked to a RAGE immunoglobulin domain such that the C-terminal amino acid of the RAGE immunoglobulin domain is linked to the N-terminal amino acid of the RAGE interdomain linker, and the C-terminal amino acid of the RAGE interdomain linker is directly linked to the N-terminal amino acid of an immunoglobulin polypeptide. The polypeptide comprising a C_{H}2 domain of an immunoglobulin may, as described above, comprise at least a fragment of a C_{H}2 domain and a C_{H}3 domain of an immunoglobulin, for example at least a fragment of the C_{H}2 and C_{H}3 domains of a human IgG1. For example, the immunoglobulin polypeptide may comprise a fragment of a C_{H}2 domain from which the hinge region has been at least partly removed.

In one embodiment, the RAGE fusion protein may comprise two immunoglobulin domains derived from RAGE protein and two immunoglobulin domains derived from a human Fc polypeptide. The RAGE fusion protein may comprise a first RAGE immunoglobulin domain and a first RAGE interdomain linker linked to a second RAGE immunoglobulin domain and a second RAGE interdomain linker, such that the N-terminal amino acid of the first interdomain linker is linked to the C-terminal amino acid of the first RAGE immunoglobulin domain, the N-terminal amino acid of the second RAGE immunoglobulin domain is linked to C-terminal amino acid of the first interdomain linker, the N-terminal amino acid of the second interdomain linker is linked to C-terminal amino acid of the second RAGE immunoglobulin domain, and the C-terminal amino acid of the RAGE second interdomain linker is linked to the N-terminal amino acid of the C_{H}2 immunoglobulin domain. In a further embodiment, the C-terminal amino acid of the RAGE second interdomain linker is directly linked to the N-terminal amino acid of the C_{H}2 immunoglobulin domain. Examples of such constructs are provided as SEQ ID NOs: 32, 33, 34 and 56 (FIG. 4).

Alternatively, a three domain RAGE fusion protein may comprise one immunoglobulin domain derived from RAGE and two immunoglobulin domains derived from a human Fc polypeptide. For example, the RAGE fusion protein may comprise a single RAGE immunoglobulin domain linked via a RAGE interdomain linker to the N-terminal amino acid of a C_{H}2 immunoglobulin domain or a portion of a C_{H}2 immunoglobulin domain. In a further embodiment, the C-terminal amino acid of the RAGE interdomain linker is directly linked the N-terminal amino acid of the C_{H}2 immunoglobulin domain or a portion of a C_{H}2 immunoglobulin domain. Examples of such constructs are provided as SEQ ID NOs: 35, 36, 37 and 57 (FIG. 5).

A RAGE interdomain linker fragment may comprise a peptide sequence that is naturally downstream of, and thus, linked to, a RAGE immunoglobulin domain. For example, for the RAGE V domain, the interdomain linker may comprise amino acid sequences that are naturally downstream from the V domain (FIG. 1F). In an embodiment, the linker may comprise SEQ ID NO: 21, corresponding to amino acids 117-123 of full-length RAGE (SEQ ID NO: 1). Or, the linker may comprise a peptide having additional portions of the natural RAGE sequence. For example, an interdomain linker comprising several amino acids (e.g., 1-3, 1-5, or 1-10, or 1-15 amino acids) upstream and downstream of SEQ ID NO: 21 may be used. Thus, in one embodiment, the interdomain linker comprises SEQ ID NO: 23 comprising amino acids 117-136 of full-length RAGE (SEQ ID NO: 1). Or, fragments of SEQ ID NO: 21 deleting, for example, 1, 2, or 3, amino acids from either end of the linker may be used. In alternate embodiments, the linker may comprise a peptide that is at least 70%, 75%, 80%, 85%, 90%, 95%, 96%, 97%, 98%, or 99% identical to SEQ ID NO: 21 or SEQ ID NO: 23.

For the RAGE C1 domain, the linker may comprise peptide sequence that is naturally downstream of the C1 domain. In an embodiment, the linker may comprise SEQ ID NO: 22, corresponding to amino acids 222-251 of full-length RAGE (SEQ ID NO: 1). Or, the linker may comprise a peptide having additional portions of the natural RAGE sequence. For example, a linker comprising several (1-3, 1-5, or 1-10, or 1-15 amino acids) amino acids upstream and downstream of SEQ ID NO: 22 may be used. Or, fragments of SEQ ID NO: 22 may be used, deleting for example, 1-3, 1-5, or 1-10, or 1-15 amino acids from either end of the linker. For example, in one embodiment, a RAGE interdomain linker may comprise SEQ ID NO: 24, corresponding to amino acids 222-226 of full-length RAGE (SEQ ID NO: 1). In alternate embodiments, the linker may comprise a peptide that is at least 70%, 75%, 80%, 85%, 90%, 95%, 96%, 97%, 98%, or 99% identical to SEQ ID NO: 22 or SEQ ID NO: 24.

Examples of amino acid substitutions that may be used in generating various point mutations to remove either a glycosylation site and/or an enzyme cleavage site are provided herein. Thus, as described herein, glutamine (Q) may be substituted for asparagine (N). Also, lysine (K) or histidine (H) may be substituted for arginine (R). Or, in alternate embodiments, neutral amino acids, as for example, alanine (A) or threonine (T) may be substituted for arginine (R). Or, neutral amino acids may be substituted for each other, as for example serine (S) may be substituted for glycine (G).

Additionally, one of skill will recognize that individual substitutions, deletions or additions which alter, add or delete a single amino acid or a small percentage of amino acids (typically less than about 5%, more typically less than about 1%) in an encoded sequence are conservatively modified variations where the alterations result in the substitution of an amino acid with a chemically similar amino acid. Conservative substitution tables providing functionally similar amino acids are well known in the art. The following example groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Generally, a conservative substitution is a substitution in which the substituting amino acid (naturally occurring or modified) is structurally related to the amino acid being substituted, i.e., has about the same size and electronic properties as the amino acid being substituted. Thus, the substituting amino acid would have the same or a similar functional group in the side chain as the original amino acid. A “conservative substitution” may also refer to utilizing a substituting amino acid which is identical to the amino acid being substituted except that a functional group in the side chain is protected with a suitable protecting group.

Also, as is known in the art, amino acids may become chemically modified from their natural structure, either by enzymatic or non-enzymatic reaction mechanisms. For example, in one embodiment, an N-terminal glutamic acid or glutamine may cyclize, with loss of water, to form pyroglutamic acid (pyroE or pE) (Chelius et al., Anal. Chem, 78: 2370-2376 (2006) and Burstein et al., Proc. National Acad. Sci., 73:2604-2608 (1976)). Alternatively, a fusion protein having an N-terminal pyroglutamic acid could potentially be accessed through a nucleic acid sequence encoding for glutamic acid at the position in the protein that via post-translational processing becomes the N-terminus (e.g., where residue 24 of SEQ ID NO: 1 is glutamate rather than a glutamine). The fusion proteins of the present invention comprise other chemical modifications that may occur either by enzymatic or non-enzymatic reaction mechanisms either during expression and/or purification, or that may be added to assist in other aspects of protein function.

The fusion proteins of the present invention may associate either in vivo or in vitro via non-covalent interactions to form multimers. Thus, in any of the previous embodiments of the fusion protein, the fusion protein may be in the form of a monomer, a dimer, a trimer, a tetramer, or a mixture thereof.

There are various advantages that may be associated with particular embodiments of the present invention. In certain embodiments, and as discussed in more detail below, the RAGE fusion proteins of the present invention may be metabolically stable when administered to a subject relative to the corresponding fusion protein containing an unmutated wild type RAGE peptide.

Also, as discussed in more detail below, the RAGE fusion proteins of the present invention may exhibit a higher affinity binding for RAGE ligands relative to the corresponding fusion protein contain an unmutated wild type RAGE peptide. In certain embodiments, the RAGE fusion proteins of the present invention bind to RAGE ligands with affinities in the high nanomolar to low micromolar range. By binding with high affinity to physiological RAGE ligands, the RAGE fusion proteins of the present invention may be used to inhibit binding of endogenous ligands to RAGE, thereby providing a means to ameliorate RAGE-mediated diseases.

### Methods of Producing RAGE Fusion Proteins

The present invention also comprises a method to make a RAGE fusion protein of any one of the fusion protein embodiments described above. Thus, in one embodiment, the present invention comprises expressing a nucleic acid sequence that encodes for any one of the fusion protein embodiments described herein.

The nucleic acid constructs of the present invention may encode RAGE fusion proteins having each of the embodiments as described herein. For example, in certain embodiments, the present invention comprises an isolated nucleic acid encoding a fusion protein comprising a RAGE polypeptide linked to an immunoglobulin polypeptide, wherein the RAGE polypeptide comprises a fragment of a mammalian RAGE having a ligand binding domain, and wherein the fusion protein comprises at least one mutation relative to the wild-type sequence in at least one of the RAGE polypeptide or the immunoglobulin polypeptide relative to the wild type sequence, wherein the mutation removes and/or alters at least one of a glycosylation site or an enzyme cleavage site. In certain embodiments, the immunoglobulin polypeptide comprises at least a fragment of a C_{H}2 domain. In certain embodiments, the C_{H}2 domain or fragment thereof does not include a hinge region. Also, in certain embodiments, the enzyme cleavage site is a furin cleavage site.

The RAGE fusion protein may be engineered by recombinant DNA techniques. For example, in one embodiment, the present invention may comprise a nucleic acid sequence comprising, complementary to, or having significant identity with, a polynucleotide sequence that encodes for any one of the fusion protein embodiments described above. For example, the linked RAGE polypeptide and the immunoglobulin polypeptide may be encoded by a recombinant DNA construct. The method may further comprise the step of incorporating the DNA construct into an expression vector. Also, the method may comprise the step of inserting the expression vector into a host cell.

Each of the embodiments described above for the various RAGE fusion proteins of the invention are included in the nucleic acid constructs, expression vectors, and host cells used to produce such RAGE fusion proteins.

Thus, embodiments of the present invention may comprise nucleic acid molecules that encode the RAGE fusion proteins of the present invention. In certain embodiments, present invention provides a nucleic acid molecule that encodes for one of the amino acid sequences of SEQ ID NOs: 62 to 81, 92 to 190, 58 to 61, 82 or, 200, 201.

In other embodiments, the nucleic acid molecules encode for a RAGE fusion protein that comprises an amino acid sequence at least 70%, 75%, 80%, 85%, 90%, 95%, 96%, 97%, 98%, or 99% identical to one of the RAGE fusion proteins of Table 1, i.e., SEQ ID NOS: 62 to 81, 92 to 190, 58 to 61, 82 or 201, wherein the RAGE polypeptide portion of the RAGE fusion protein comprises: (i) a point mutation or mutations at one or more of residues to remove and/or alter a glycosylation site; and/or (ii) a point mutation at one or more of residues to remove and/or alter a furin cleavage site.

In another embodiment, the nucleic acid encodes a fusion protein comprising an immunoglobulin polypeptide wherein the immunoglobulin polypeptide comprises a point mutation to remove and/or alter a glycosylation site in the immunoglobulin polypeptide relative to the native immunoglobulin amino acid sequence.

The point mutation may be selected to provide a beneficial effect in the properties of the RAGE fusion protein. For example, in certain embodiments, the point mutation changes the sequence in the wild-type RAGE polypeptide present at a glycosylation site. In this way, the type and/or extent of glycosylation may be modified.

The glycosylation site may be one of various types of glycosylation sites that are found in proteins. For example, in certain embodiments, the glycosylation recognition site is NXS or NXT. In certain embodiments, X is not proline. Thus, in certain embodiments, the point mutation changes the sequence in the wild-type RAGE polypeptide from NIT to QIT and/or from NGS to QGS, and/or from NGS to NSS and/or from NST to QST to remove and/or alter at least one glycosylation site. In some embodiments, the glycosylation site is located in the RAGE portion of the RAGE fusion protein. For example, the glycosylation site may, in certain embodiments, be located within the ligand binding site or the ligand binding domain. Additionally and/or alternatively, the glycosylation site is located in the immunoglobulin portion of the RAGE fusion protein. Or, other glycosylation sites that may exist in the RAGE fusion protein may be targeted.

In certain embodiments, the enzyme cleavage site is a furin cleavage site, such that the point mutation changes the sequence in the wild-type RAGE polypeptide present at a recognition site for furin cleavage of the RAGE polypeptide. For example, in alternate embodiments, the point mutation changes the sequence in the wild-type RAGE polypeptide from one of: (i) PRHRALR to PRHAALR; (ii) PRHRALR to PRHKALR; (iii) PRHRALR to PRHRALA; (iv) PRHRALR to PRHRALK; (v) PRHRALR to PRHRALH; (vi) PRHRALR to PRHRALT; (vii) PRHRALR to PRHHALR; or (viii) PRHRALR to PRHTALR to remove and/or alter a furin cleavage site.

In other embodiments, the nucleic acid comprises a nucleic acid sequence at least 70%, 75%, 80%, 85%, 90%, 95%, 96%, 97%, 98%, or 99% identical to SEQ ID NO: 202, 203, 204, 205, 206, 207, 208, 209, 210, 211, 212, 213, 214, 215, 216, 217, 218, 219, or 220, wherein the nucleic acid sequence encodes for a fusion protein wherein the RAGE polypeptide portion of the fusion protein comprises: (i) a point mutation or mutations at one or more of residues to remove and/or alter a glycosylation site; and/or (ii) a point mutation at one or more of residues to remove and/or alter a furin cleavage site. In another embodiment, the present invention provides a nucleic acid sequence comprising SEQ ID NO: 202, 203, 204, 205, 206, 207, 208, 209, 210, 211, 212, 213, 214, 215, 216, 217, 218, 219, or 220.

As described in more detail herein, the RAGE fusion proteins of the present invention may be expressed in cells that are cultured so as to produce purified preparations of the protein. FIG. 7 provides nucleic acid sequences that may be used to encode a RAGE fusion protein of the present invention, wherein SEQ ID NO: 221 comprises a wild-type nucleic acid sequence (i.e., wild type for human RAGE and human immunoglobulin) and a nucleic acid sequence (SEQ ID NO: 222) optimized for cell culture expression. Also, FIG. 8A provides wild-type and modified nucleic acid sequences (SEQ ID NOs: 84 and 85) corresponding to the signal sequence of human RAGE.

Thus, in certain embodiments, the nucleic acid molecules encoding for the RAGE polypeptides of the invention further encode for signal sequences. As described elsewhere herein, the RAGE polypeptide may comprise the native signal sequence (SEQ ID NO: 83) or signal sequences that have been modified from the native sequence for improved expression in cell culture. Accordingly, the nucleic acid molecules encoding for the RAGE polypeptides of the invention may further comprise the native nucleic acid signal sequence (SEQ ID NO: 84) or nucleic acid signal sequences that have been modified from the native nucleic acid sequence (e.g., SEQ ID NO: 85).

The fusion protein of the present invention may comprise at least a fragment of a C_{H}2 and C_{H}3 domain of an immunoglobulin. In one embodiment, the fragment of a C_{H}2 and C_{H}3 domain of an immunoglobulin comprises SEQ ID NO: 38. The immunoglobulin peptide may be encoded by the nucleic acid sequence of SEQ ID NO: 39 or SEQ ID NO: 41. The immunoglobulin sequence in SEQ ID NO: 38 or SEQ ID NO: 40 may also be encoded by SEQ ID NO: 52 or SEQ ID NO: 53, where silent base changes for the codons that encode for proline (CCG to CCC) and glycine (GGT to GGG) at the C-terminus of the sequence remove a cryptic RNA splice site near the terminal codon (i.e., nucleotides 622-627 of SEQ ID NO: 39 are modified to generate SEQ ID NO: 52 or nucleotides 652-657 of SEQ ID NO: 41 are modified to generate SEQ ID NO: 53).

As described above, the RAGE fusion protein may be encoded by a recombinant DNA construct and the method may comprise the step of incorporating the DNA construct into an expression vector. Thus, embodiments of the present invention also comprise expression vectors encoding nucleic acids that encode the RAGE fusion proteins of the present invention. Also, the method may comprise transfecting the expression vector into a host cell. The expression vectors and transfected host cells of the present invention may encode RAGE fusion proteins having each of the embodiments as described herein.

Thus, in certain embodiments, the invention comprises an expression vector comprising an isolated nucleic acid encoding a fusion protein comprising a RAGE polypeptide linked to an immunoglobulin polypeptide, wherein the RAGE polypeptide comprises a fragment of a mammalian RAGE having a ligand binding domain, and where the fusion protein comprises at least one mutation in at least one of the RAGE polypeptide or the immunoglobulin polypeptide relative to the wild-type sequence, wherein the mutation removes and/or alters at least one of a glycosylation site or an enzyme cleavage site. In certain embodiments, the immunoglobulin polypeptide comprises at least a fragment of a C_{H}2 domain. Also, in certain embodiments, the C_{H}2 domain or fragment thereof does not include the hinge region. Also, in certain embodiments, the enzyme cleavage site is a furin cleavage site.

Yet other embodiments of the present invention also comprise cells transfected with an expression vector encoding nucleic acid sequences that encode the RAGE fusion proteins of the present invention. Thus, in certain embodiments, the present invention comprises a host cell transfected with an expression vector comprising an isolated nucleic acid encoding a fusion protein comprising a RAGE polypeptide linked to an immunoglobulin polypeptide, wherein the RAGE polypeptide comprises a fragment of a mammalian RAGE having a ligand binding domain, and where the fusion protein has at least one mutation in at least one of the RAGE polypeptide or the immunoglobulin polypeptide relative to the wild-type sequence, wherein the mutation removes and/or alters at least one of a glycosylation site or an enzyme cleavage site. In some embodiments, the immunoglobulin polypeptide comprises at least a fragment of a C_{H}2 domain. Also, in certain embodiments, the C_{H}2 domain or fragment thereof does not include the hinge region. Also, in certain embodiments, the enzyme cleavage site is a furin cleavage site.

With respect to methods of the invention comprising the incorporation of DNA constructs into an expression vector, plasmids may be constructed to express RAGE-IgG fusion proteins by fusing different lengths of a 5′ cDNA sequence of human RAGE with a 3′ cDNA sequence of a human immunoglobulin polypeptide (e.g., IgG1 Fc (γ1)). The expression cassette sequences may be inserted into an expression vector such as pcDNA3.1 expression vector (Invitrogen, CA) using standard recombinant techniques. Or, other vectors may be used.

With respect to methods of the invention comprising the transfection of an expression vector into a host cell, RAGE fusion proteins may be expressed in mammalian expression systems, including systems in which the expression constructs are introduced into the mammalian cells using virus such as retrovirus, adenovirus or baculovirus. Mammalian cell lines available as hosts for expression are well known in the art and include many immortalized cell lines available from the American Type Culture Collection (ATCC). These include, inter alia, Chinese hamster ovary (CHO) cells, Human Embryonic Kidney (HEK) 293, NS0, SP2 cells, HeLa cells, baby hamster kidney (BHK) cells, monkey kidney cells (COS), human hepatocellular carcinoma cells (e.g., Hep G2), A549 cells, and a number of other cell lines. In certain embodiments, either CHO or HEK 293 cells are used.

Cell lines may be selected by determining which cell lines have high expression levels of a RAGE fusion protein. Other cell lines that may be used are insect cell lines, such as Sf9 or Sf21 cells. Plant host cells include, e.g., Nicotiana, Arabidopsis, duckweed, corn, wheat, potato, etc. Bacterial host cells include E. coli and Streptomyces species. Yeast host cells include Schizosaccharomyces pombe, Saccharomyces cerevisiae and Pichia pastoris. When recombinant expression vectors encoding RAGE fusion protein genes are introduced into mammalian host cells, the RAGE fusion proteins are produced by culturing the host cells for a period of time sufficient to allow for expression of the RAGE fusion protein in the host cells or secretion of the RAGE fusion protein into the culture medium in which the host cells are grown. RAGE fusion proteins may be recovered from the culture medium using standard protein purification methods.

Nucleic acid molecules encoding RAGE fusion proteins and expression vectors comprising these nucleic acid molecules may be used for transfection of a suitable mammalian, plant, bacterial or yeast host cell. Transformation may be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. Additionally, commercially available transfection agents (e.g., Fugene 6, available from Roche Molecular Biochemicals and Invitrogen) may be used.

In addition, nucleic acid molecules may be introduced into mammalian cells by viral vectors. Methods of transforming plant cells are well known in the art, including, e.g., Agrobacterium-mediated transformation, biolistic transformation, direct injection, electroporation and viral transformation. Methods of transforming bacterial and yeast cells are also well known in the art.

An expression vector may also be delivered to an expression system using DNA biolistics, wherein the plasmid is precipitated onto microscopic particles, preferably gold, and the particles are propelled into a target cell or expression system. DNA biolistics techniques are well-known the art and devices, e.g., a “gene gun”, are commercially available for delivery of the microparticles in to a cell (e.g., Helios Gene Gun, Bio-Rad Labs., Hercules, Calif.) and into the skin (PMED Device, PowderMed Ltd., Oxford, UK).

Expression of RAGE fusion proteins from production cell lines may be enhanced using a number of known techniques. For example, the glutamine synthetase gene expression system (the GS system) and the plasma-encoded neomycin resistance system are common approaches for enhancing expression under certain conditions.

In certain embodiments, a recombinant expression vector may be transfected into Chinese Hamster Ovary cells (CHO) or HEK cells and expression optimized. In alternate embodiments, the cells may produce 0.1 to 20 grams/liter, or 0.5 to 10 grams/liter, or about 1-2 grams/liter protein.

As is known in the art, such nucleic acid constructs may be modified by mutation, as for example, by PCR amplification of a nucleic acid template with primers comprising the mutation of interest. In this way, polypeptides comprising varying affinity for RAGE ligands may be designed. In one embodiment, the mutated sequences may be 90% or more identical to the starting DNA. As such, variants may include nucleotide sequences that hybridize under stringent conditions (i.e., equivalent to about 20-27° C. below the melting temperature (TM) of the DNA duplex in 1 molar salt).

The coding sequence may be expressed by transfecting the expression vector into an appropriate host. For example, the recombinant vectors may be stably transfected into Chinese Hamster Ovary (CHO) cells, and cells expressing the RAGE fusion protein selected and cloned. In an embodiment, cells expressing the recombinant construct are selected for plasmid-encoded neomycin resistance by applying antibiotic G418. Individual clones may be selected and clones expressing high levels of recombinant protein as detected by Western Blot analysis of the cell supernatant may be expanded, and the gene product purified by affinity chromatography using Protein A columns.

The RAGE fusion proteins of the present invention may comprise improved in vivo stability over RAGE polypeptides not comprising the mutations described herein. The RAGE fusion protein may be further modified to increase stability, efficacy, potency and bioavailability. Thus, the RAGE fusion proteins of the present invention may be modified by post-translational processing or by chemical modification. For example, the RAGE fusion protein may be synthetically prepared to include L-, D-, or unnatural amino acids, alpha-disubstituted amino acids, or N-alkyl amino acids. Additionally, proteins may be modified by acetylation, acylation, ADP-ribosylation, amidation, attachment of lipids such as phosphatidyinositol, formation of disulfide bonds, and the like. Furthermore, polyethylene glycol can be added to increase the biological stability of the RAGE fusion protein.

### Binding of RAGE Antagonists to RAGE Fusion Proteins

The RAGE fusion proteins of the present invention may be used in a number of applications. For example, the RAGE fusion protein of the present invention may be used in a binding assay to identify RAGE ligands, such as RAGE agonists, antagonists, or modulators.

For example, in one embodiment, the present invention provides a method for detection of RAGE modulators comprising: (a) providing a RAGE fusion protein of any one of the above embodiments; (b) mixing a compound of interest and a ligand having a known binding affinity for RAGE with the RAGE fusion protein; and (c) measuring binding of the known RAGE ligand to the RAGE fusion protein in the presence of the compound of interest.

The RAGE fusion proteins may also be used in a kit for the detection of RAGE modulators. For example, in one embodiment, a kit of the present invention may comprise: (a) a compound having known binding affinity to RAGE as a positive control; (b) a RAGE fusion protein of any one of the above embodiments; and (c) instructions for use.

For example, the RAGE fusion protein may be used in a binding assay to identify potential RAGE ligands essentially as is described in U.S. Pat. No. 6,908,741 incorporated by reference in its entirety herein. In one example embodiment of such a binding assay, a known RAGE ligand may coated onto a solid substrate (e.g., Maxisorb plates) at a concentration of about 5 micrograms per well, where each well contains a total volume of about 100 microliters (μL). The plates may be incubated at 4° C. overnight to allow the ligand to absorb or bind to the substrate. Alternatively, shorter incubation periods at higher temperature (e.g., room temperature) may be used. After a period of time to allow for the ligand to bind to the substrate, the assay wells may be aspirated and a blocking buffer (e.g., 1% BSA in 50 mM imidazole buffer, pH 7.2) may be added to block nonspecific binding. For example, blocking buffer may be added to the plates for 1 hour at room temperature. The plates may then be aspirated and/or washed with a wash buffer. In one embodiment, a buffer comprising 20 mM Imidazole, 150 mM NaCl, 0.05% Tween-20, 5 mM CaCl₂ and 5 mM MgCl₂, pH 7.2 may be used as a wash buffer. The RAGE fusion protein may then added at increasing dilutions to the assay wells. The RAGE fusion protein may then be allowed to incubate with the immobilized ligand in the assay well such that binding can attain equilibrium. In one embodiment, the RAGE fusion protein is allowed to incubate with the immobilized ligand for about one hour at 37° C. In alternate embodiments, longer incubation periods at lower temperatures may be used. After the RAGE fusion protein and immobilized ligand have been incubated, the plate may be washed to remove any unbound RAGE fusion protein. The RAGE fusion protein bound to the immobilized ligand may be detected in a variety of ways. In one embodiment, detection employs an ELISA. Thus, in one embodiment, an immunodetection complex containing a monoclonal mouse anti-human IgG1, biotinylated goat anti-mouse IgG, and an avidin linked alkaline phosphatase may be added to the RAGE fusion protein immobilized in the assay well. The immunodetection complex may be allowed to bind to the immobilized RAGE fusion protein such that binding between the RAGE fusion protein and the immunodetection complex attains equilibrium. For example, the complex may be allowed to bind to the RAGE fusion protein for one hour at room temperature. At that point, any unbound complex may be removed by washing the assay well with wash buffer. The bound complex may be detected by adding the alkaline phosphatase substrate, para-nitrophenylphosphate (PNPP), and measuring conversion of PNPP to para-nitrophenol (PNP) as an increase in absorbance at 405 nm.

The binding assay of the present invention may be used to quantify ligand binding to RAGE. In an embodiment, RAGE ligands bind to the RAGE fusion protein with nanomolar (nM) or micromolar (μM) affinity. In alternate embodiments, RAGE ligands may bind to the RAGE fusion protein of the present invention with binding affinities ranging from 0.1 to 1000 nanomolar (nM), or from 1 to 500 nM, or from 10 to 80 nM, or ranges within these ranges.

### Modulation of Cellular Effectors

Embodiments of the RAGE fusion proteins of the present invention may be used to modulate a biological response mediated by RAGE. For example, the RAGE fusion proteins may be designed to modulate RAGE-induced increases in gene expression. Thus, in an embodiment, RAGE fusion proteins of the present invention may be used to modulate the function of biological enzymes. For example, the interaction between RAGE and its ligands may generate oxidative stress and activation of NF-κB, and NF-κB regulated genes, such as the cytokines IL-1β, TNF-α, and the like. In addition, several other regulatory pathways, such as those involving p21ras, MAP kinases, ERK1, and ERK2, have been shown to be activated by binding of AGEs and other ligands to RAGE.

### Physiological Characteristics of RAGE Fusion Proteins

While sRAGE can have a therapeutic benefit in the modulation of RAGE-mediated diseases, human sRAGE may have limitations as a stand-alone therapeutic based on the relatively short half-life of sRAGE in plasma. For example, whereas rodent sRAGE has a half-life in normal and diabetic rats of approximately 20 hours, human sRAGE has a half-life of less than 2 hours when assessed by retention of immunoreactivity sRAGE (Renard et al., J. Pharmacol. Exp. Ther., 290:1458-1466 (1999)).

1. Modification of Immunoglobulin Hinge

To generate a RAGE therapeutic that has similar binding characteristics as sRAGE, but a more stable pharmacokinetic profile, a RAGE fusion protein comprising a RAGE ligand binding site linked to one or more immunoglobulin domains may be used. As is known in the art, the immunoglobulin domains may include the Fc portion of the immunoglobulin heavy chain.

The immunoglobulin Fc portion may confer several attributes to a RAGE fusion protein. For example, the Fc fusion protein may increase the serum half-life of such fusion proteins, often from hours to several days. The increase in pharmacokinetic stability is generally a result of the interaction of the linker between C_{H}2 and C_{H}3 regions of the Fc fragment with the FcRn receptor (Wines et al., J. Immunol., 164:5313-5318 (2000)).

Although a fusion protein comprising an immunoglobulin Fc polypeptide may provide the advantage of increased stability, such fusion proteins may elicit an inflammatory response when introduced into a host. The inflammatory response may be due, in large part, to the Fc portion of the immunoglobulin of the fusion protein. The proinflammatory response may be a desirable feature if the target is expressed on a diseased cell type that needs to be eliminated (e.g., a cancer cell, and/or a population of lymphocytes causing an autoimmune disease). The proinflammatory response may be a neutral feature if the target is a soluble protein, as most soluble proteins do not activate immunoglobulins. However, the proinflammatory response may be a negative feature if the target is expressed on cell types whose destruction would lead to untoward side-effects. Also, the proinflammatory response may be a negative feature if an inflammatory cascade is established at the site of a fusion protein binding to a tissue target, since many mediators of inflammation may be detrimental to surrounding tissue, and/or may cause systemic effects.

The primary proinflammatory site on immunoglobulin Fc fragments resides on the hinge region between the C_{H}1 and C_{H}2. This hinge region interacts with the FcR1-3 on various leukocytes and trigger these cells to attack the target. (Wines et al., J. Immunol., 164:5313-5318 (2000)).

As therapeutics for RAGE-mediated diseases, RAGE fusion proteins may not require the generation of an inflammatory response. Thus, embodiments of the RAGE fusion proteins of the present invention may comprise a RAGE fusion protein comprising a RAGE polypeptide linked to an immunoglobulin domain(s) where the Fc hinge region from the immunoglobulin is removed and replaced with a RAGE polypeptide. In this way, interaction between the RAGE fusion protein and Fc receptors on inflammatory cells may be minimized. It may be important, however, to maintain proper stacking and other three-dimensional structural interactions between the various immunoglobulin domains of the RAGE fusion protein. Thus, embodiments of the RAGE fusion proteins of the present invention may substitute the biologically inert, but structurally similar RAGE interdomain linker that separates the V and C1 domains of RAGE, or the linker that separates the C1 and C2 domains of RAGE, in lieu of the normal hinge region of the immunoglobulin heavy chain. Thus, the RAGE polypeptide of the RAGE fusion protein may comprise an interdomain linker sequence that is naturally found downstream of a RAGE immunoglobulin domain to form a RAGE immunoglobulin domain/linker fragment. In this way, the three dimensional interactions between the immunoglobulin domains contributed by either RAGE or the immunoglobulin may be maintained.

In an embodiment, a RAGE fusion protein of the present invention may comprise a substantial increase in pharmacokinetic stability as compared to sRAGE or as compared to a similar wild type RAGE fusion protein. Thus, in an embodiment, the RAGE fusion proteins of the present invention may be used to antagonize binding of physiological ligands to RAGE as a means to treat RAGE-mediated diseases without generating an unacceptable amount of inflammation. The RAGE fusion proteins of the present invention may exhibit a substantial decrease in generating a proinflammatory response as compared to IgG. Further, the RAGE fusion protein of the present invention may exhibit increased plasma concentration over time relative to a similar wild type RAGE fusion protein.

2. Mutations to Remove Glycosylation and Furin Cleavage

a. Pharmacokinetics

In certain embodiments, the RAGE fusion proteins of the present invention has at least one mutation relative to the wild-type sequence in at least one of the RAGE polypeptide or the immunoglobulin polypeptide, wherein the mutation removes and/or alters at least one of a glycosylation site or an enzyme cleavage site. For example, the mutation or mutations may remove a furin cleavage site. Additionally and/or alternatively, the mutation or mutations may remove a glycosylation site.

Furin cleavage has the potential to cleave a RAGE fusion protein at about position 193-198 of a fusion protein comprising two RAGE domains (e.g., having an amino acid sequence as shown in SEQ ID NOs: 32, 33, 34 and 56). As such, in certain embodiments, mutation of a furin cleavage site may be expected to improve the pharmacokinetic profile of the RAGE fusion protein as compared to a wild-type sequence.

Also, mutation of a glycosylation site in RAGE and/or the immunoglobulin portion of the fusion protein may be able to affect either the stability and/or the pharmacokinetic profile of the protein. As such, in certain embodiments, mutation of a glycosylation site may improve the pharmacokinetic profile of the RAGE fusion protein as compared to a wild-type sequence.

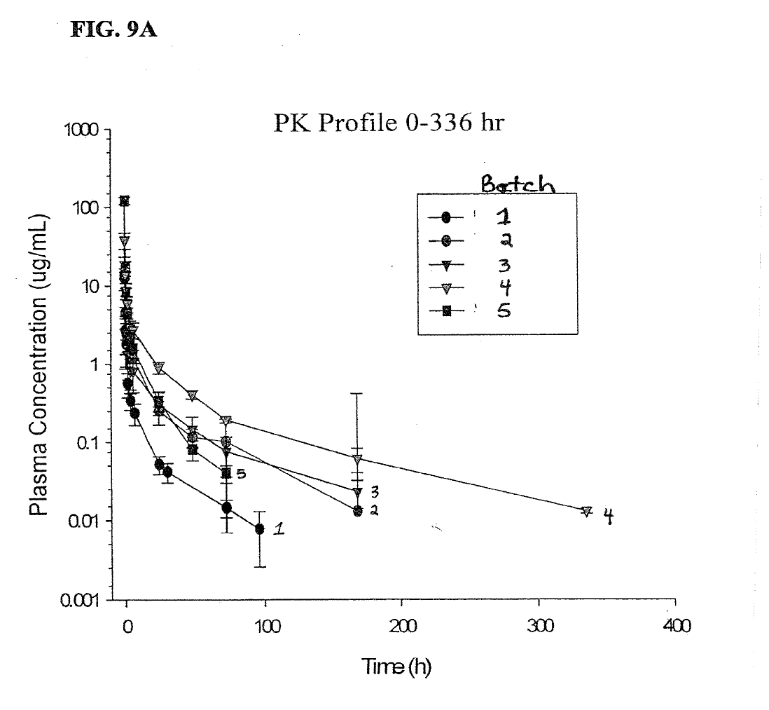

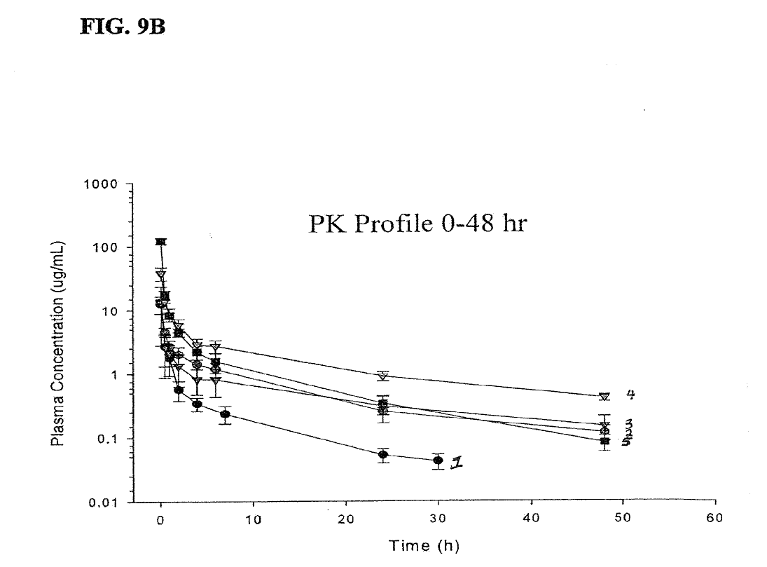

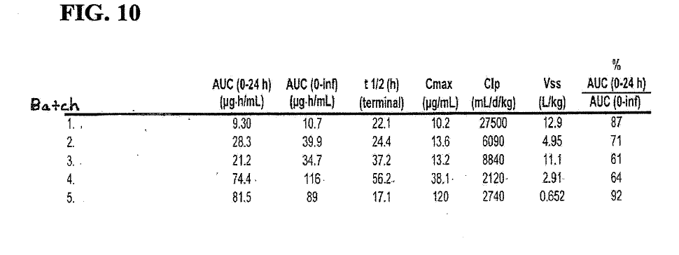

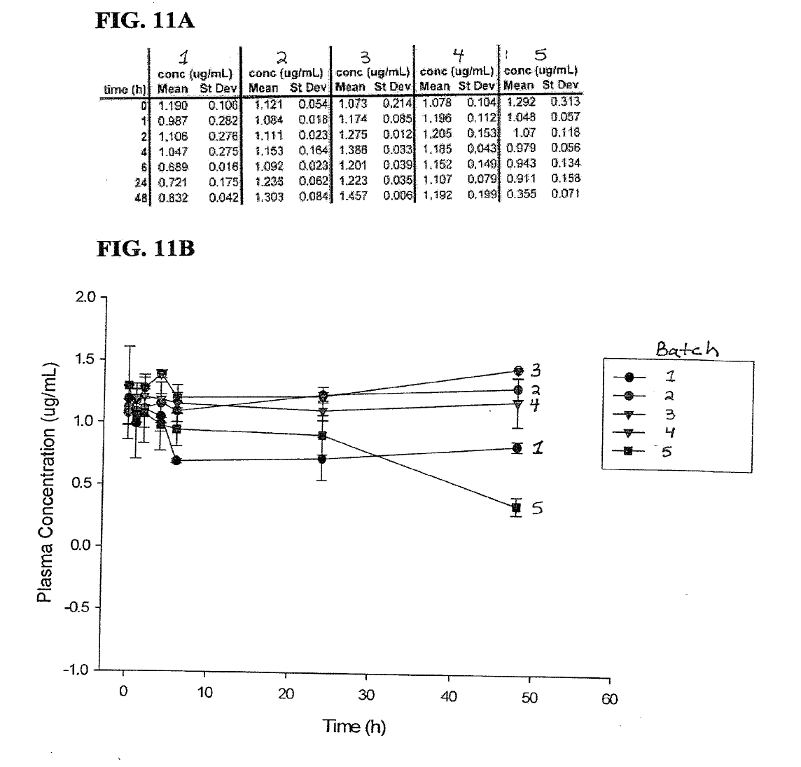

FIGS. 9-11 illustrate how embodiments of the RAGE fusion proteins of the invention may have improved pharmacokinetic profiles as compared to analogous RAGE fusion proteins having the wild-type sequence. The data in FIGS. 9-10 show the pharmacokinetics and stability of RAGE fusion proteins of the invention in mice after intravenous administration of RAGE fusion proteins at 10 mg/kg. The data in FIG. 11 show in vitro stability of RAGE fusion proteins in plasma. The RAGE fusion proteins used were as follows:

Batch 1—SEQ ID NO: 56 from CHO cells (wild type);

Batch 2—SEQ ID NO: 56 from HEK cells (wild type);

Batch 3—SEQ ID NO: 67 from HEK cells (R198A);

Batch 4—SEQ ID NO: 71 from HEK cells (R198A and N2Q); and

Batch 5—SEQ ID NO: 72 from HEK cells (R198A and N58Q).

As shown in FIG. 9, RAGE fusion proteins having the R198A (Batch 3) and the R198A and N2Q mutations (Batch 4) may remain at higher plasma concentrations over time as compared to other tested RAGE fusion proteins (FIG. 9A and FIG. 9B). This effect may be observed over time periods from 0-336 hours (FIG. 9A) and 0-48 hours (FIG. 9B).

FIG. 10 shows mean pharmacokinetic parameters for the tested RAGE fusion proteins of Batches 1-5 following intravenous administration to mice (10 mg/kg). It can be seen that overall drug exposure as assessed by the area under the curve (AUC) may be greater in the RAGE fusion proteins having the R198A and N2Q mutations (Batch 4) and the R198A and N58Q mutations (Batch 5) than for wild-type RAGE fusion proteins (Batches 1 and 2) and RAGE fusion proteins having just the R198A mutation (Batch 3).

Also, in certain embodiments, the total body clearance (Clp) for the RAGE fusion proteins having mutations as compared to wild-type RAGE fusion proteins may differ. Thus, it can be seen that RAGE fusion proteins having the R198A and N2Q mutations (Batch 4) and the R198A and N58Q mutations (Batch 5) have lower total body clearance than for the wild-type RAGE fusion proteins (Batches 1 and 2) and RAGE fusion proteins having just the R198A mutation (Batch 3). Also, in certain embodiments, the steady state volume of distribution (Vss) for the RAGE fusion proteins having the R198A and N2Q mutations (Batch 4) and the R198A and N58Q mutations (Batch 5) may be lower than for Batches 1 and 2 (wild type) and Batch 3 (R198A).

The higher plasma concentration in vivo may be due to various factors. One factor may be the stability of the fusion protein in plasma (i.e., as compared to active uptake and/or clearance of the RAGE fusion protein). FIG. 11 shows in vitro stability data for the tested RAGE fusion proteins of Batches 1 to 5 described above. It can be seen that in certain embodiments, a RAGE fusion proteins having the R198A and N2Q mutations (Batch 4) has increased stability in plasma as compared to either the wild-type RAGE fusion proteins (Batches 1 and 2) or RAGE fusion proteins having the R198A mutation and, at longer time points, the N58Q and R198A mutations (Batch 5).

Thus, in certain embodiments, RAGE fusion proteins having both a mutation to remove a furin cleavage site (e.g., R198A) and a mutation to remove and/or reduce glycosylation in the ligand binding domain and/or ligand binding site of RAGE (e.g., N2Q and/or N58Q) may provide improved pharmacokinetic profiles as compared to analogous proteins having the wild-type sequence.

b. Ligand Binding

RAGE fusion proteins may act as therapeutic agents by binding to ligands that can interact with RAGE in vivo so as to reduce the potentially deleterious effect(s) of RAGE a ligand(s) in a subject. For example, as discussed in more detail below, RAGE fusion proteins having a ligand binding domain can bind to amyloid-beta so as to reduce, and in some embodiments reverse, plaque formation in Alzheimer's Disease.

Glycosylation of RAGE has the potential to reduce ligand binding (see e.g., Owasa et al., Biochimica et Biophysica Acta, 1770: 1468-1474 (2007)). Initial studies with wild-type RAGE fusion proteins (e.g., SEQ ID NOs: 32, 33, 34 and 56) cultured under various conditions indicated that RAGE fusion proteins cultured under conditions to reduce glycosylation, e.g., in the presence of tunicamycin (to inhibit the glycosyltransferase that transfers N-acetylglucosamine-1-phosphate (P-GlcNAc) from UDP-N-acetylglucosamine to dolichol phosphate in the first step of glycoprotein synthesis), and/or grown in 2-deoxy-D-glucose (e.g., to inhibit hexokinase and glucose phosphoisomerase so as to reduce glycosylation mediated by these enzymes), could result in an increase of RAGE fusion protein having an increase in 0-glycan and 1-glycan occupancy with a loss of 2-glycan and 3-glycan occupancy as measured by reverse phase high pressure liquid chromatography (RP-HPLC) and liquid chromatography mass spectrometry (LC-MS). For example, it was found that for RAGE fusion protein expressed from cells cultured in the presence of tunicamycin, there was a decrease in glycosylation from a population of RAGE fusion proteins exhibiting about 67% 3-glycan and 22% 2 glycan, to a population of RAGE fusion proteins exhibiting about 42% 3-glycan, 16% 2-glycan, and 31% 0-glycan. When RAGE fusion proteins were isolated from RP-HPLC peaks corresponding to either the 3-glycan, 2-glycan or 0 glycan RAGE fusion proteins, it was found that the 0-glycan RAGE fusion protein exhibited an increase in binding of 529% as compared to the control RAGE fusion protein (i.e., mixture of 67% 3-glycan and 22% 2 glycan RAGE fusion protein) having a relative binding of 100% (data not shown). The increase in binding found for the 0-glycan RAGE fusion protein was even greater when compared to binding seen with the 3 glycan fraction (i.e., about a 10× increase in binding) (data not shown).

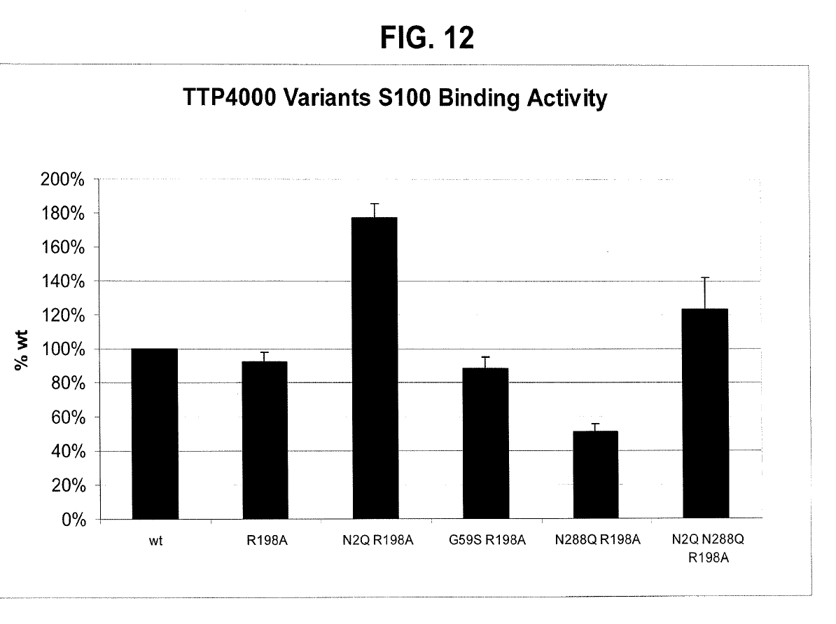

FIG. 12 provides data showing the relative binding for various RAGE fusion protein mutants. It can be seen that the N2Q mutation of the RAGE fusion protein significantly improves binding of ligands (S100b) to the RAGE fusion protein as shown for both the N2Q, R198A mutant and the N2Q, N288Q, R198A mutants. Interestingly, the N2Q, R198A mutant was the construct that displayed significantly improved pharmacokinetic half-life and stability in plasma (FIGS. 9-11).

Treatment of Disease with RAGE Fusion Proteins

The present invention also comprises methods for the treatment of RAGE-mediated disorder in a human subject. In an embodiment, the method may comprise administering to a subject a RAGE fusion protein comprising a RAGE fusion protein of any one of the previous embodiments.

The RAGE fusion protein may be administered as a pharmaceutical formulation. The pharmaceutical formulation may comprise a RAGE fusion protein, a lyoprotectant, and a buffer.

A variety of animal models have been used to validate the use of compounds that modulate RAGE as therapeutics. Examples of these models are as follows:
a) sRAGE inhibited neointimal formation in a rat model of restenosis following arterial injury in both diabetic and normal rats by inhibiting endothelial, smooth muscle and macrophage activation via RAGE (Zhou et al., Circulation 107:2238-2243 (2003));
b) Inhibition of RAGE/ligand interactions, using either sRAGE or an anti-RAGE antibody, reduced amyloid plaque formation in a mouse model of systemic amyloidosis (Yan et al., Nat. Med., 6:643-651 (2000)). Accompanying the reduction in amyloid plaques was a reduction in the inflammatory cytokines, interleukin-6 (IL-6) and macrophage colony stimulating factor (M-CSF) as well as reduced activation of NF-kB in the treated animals;
c) RAGE transgenic mice (RAGE overexpressers and RAGE dominant negative expressers) exhibit plaque formation and cognitive deficits in a mouse model of AD (Arancio et al., EMBO J., 23:4096-4105 (2004));
d) Treatment of diabetic rats with sRAGE reduced vascular permeability (Bonnardel-Phu et al., Diabetes, 48:2052-2058 (1999));
e) Treatment with sRAGE reduced atherosclerotic lesions in diabetic apolipoprotein E-null mice and prevented the functional and morphological indices of diabetic nephropathy in db/db mice (Hudson et al., Arch. Biochem. Biophys., 419:80-88 (2003)); and
f) sRAGE attenuated the severity of inflammation in a mouse model of collagen-induced arthritis (Hofmann et al., Genes Immunol., 3:123-135 (2002)), a mouse model of experimental allergic encephalomyelitis (Yan et al., Nat. Med. 9:28-293 (2003)) and a mouse model of inflammatory bowel disease (Hofmann et al., Cell, 97:889-901 (1999)).

Thus, in an embodiment, the RAGE fusion proteins of the present invention may be used to treat a symptom of diabetes and/or complications resulting from diabetes mediated by RAGE. In alternate embodiments, the symptom of diabetes or diabetic late complications may comprise diabetic nephropathy, diabetic retinopathy, a diabetic foot ulcer, a cardiovascular complication of diabetes, or diabetic neuropathy.

Originally identified as a receptor for molecules whose expression is associated with the pathology of diabetes, RAGE itself is essential to the pathophysiology of diabetic complications. In vivo, inhibition of RAGE interaction with its ligand(s) has been shown to be therapeutic in multiple models of diabetic complications and inflammation (Hudson et al., Arch. Biochem. Biophys., 419:80-88 (2003)). For example, a two-month treatment with anti-RAGE antibodies normalized kidney function and reduced abnormal kidney histopathology in diabetic mice (Flyvbjerg et al., Diabetes 53:166-172 (2004)). Furthermore, treatment with a soluble form of RAGE (sRAGE) which binds to RAGE ligands and inhibits RAGE/ligand interactions, reduced atherosclerotic lesions in diabetic apolipoprotein E-null mice and attenuated the functional and morphological pathology of diabetic nephropathy in db/db mice (Bucciarelli et al., Circulation 106:2827-2835 (2002)).

Also, it has been shown that nonenzymatic glycoxidation of macromolecules ultimately resulting in the formation of advanced glycation endproducts (AGEs) is enhanced at sites of inflammation, in renal failure, in the presence of hyperglycemia and other conditions associated with systemic or local oxidant stress (Dyer et al., J. Clin. Invest., 91:2463-2469 (1993); Reddy et al., Biochem., 34:10872-10878 (1995); Dyer et al., J. Biol. Chem., 266:11654-11660 (1991); Degenhardt et al., Cell Mol. Biol., 44:1139-1145 (1998)). Accumulation of AGEs in the vasculature can occur focally, as in the joint amyloid composed of AGE-β₂-microglobulin found in patients with dialysis-related amyloidosis (Miyata et al., J. Clin. Invest., 92:1243-1252 (1993); Miyata et al., J. Clin. Invest., 98:1088-1094 (1996)), or generally, as exemplified by the vasculature and tissues of patients with diabetes (Schmidt et al., Nature Med., 1:1002-1004 (1995)). The progressive accumulation of AGEs over time in patients with diabetes suggests that endogenous clearance mechanisms are not able to function effectively at sites of AGE deposition. Such accumulated AGEs have the capacity to alter cellular properties by a number of mechanisms. Although RAGE is expressed at low levels in normal tissues and vasculature, in an environment where the receptor's ligands accumulate, it has been shown that RAGE becomes upregulated (Li et al., J. Biol. Chem., 272:16498-16506 (1997); Li et al., J. Biol. Chem., 273:30870-30878 (1998); Tanaka et al., J. Biol. Chem., 275:25781-25790 (2000)). RAGE expression is increased in endothelium, smooth muscle cells and infiltrating mononuclear phagocytes in diabetic vasculature. Also, studies in cell culture have demonstrated that AGE-RAGE interaction causes changes in cellular properties important in vascular homeostasis.

In other embodiments, the RAGE fusion proteins of the present invention may also be used to treat or reverse amyloidoses and Alzheimer's disease. RAGE is a receptor for amyloid beta (Aβ) as well as other amyloidogenic proteins including SAA and amylin (Yan et al., Nature, 382:685-691 (1996); Yan et al., Proc. Natl. Acad. Sci., USA, 94:5296-5301 (1997); Yan et al., Nat. Med., 6:643-651 (2000); Sousa et al., Lab Invest., 80:1101-1110 (2000)). Also, the RAGE ligands, including AGEs, S100b and Aβ proteins, are found in tissue surrounding the senile plaque in man (Luth et al., Cereb. Cortex 15:211-220 (2005); Petzold et al, Neurosci. Lett., 336:167-170 (2003); Sasaki et al., Brain Res., 12:256-262 (2001; Yan et al., Restor. Neurol Neurosci., 12:167-173 (1998)). It has been shown that RAGE binds β-sheet fibrillar material regardless of the composition of the subunits (amyloid-β peptide, amylin, serum amyloid A, prion-derived peptide) (Yan et al., Nature, 382:685-691 (1996); Yan et al., Nat. Med., 6:643-651 (2000)). In addition, deposition of amyloid has been shown to result in enhanced expression of RAGE. For example, in the brains of patients with Alzheimer's disease (AD), RAGE expression increases in neurons and glia (Yan, et al., Nature 382:685-691 (1996)). Concurrent with expression of RAGE ligands, RAGE is upregulated in astrocytes and microglial cells in the hippocampus of individuals with AD but is not upregulated in individuals that do not have AD (Lue et al., Exp. Neurol., 171:29-45 (2001)). These findings suggest that cells expressing RAGE are activated via RAGE/RAGE ligand interactions in the vicinity of the senile plaque. Also, in vitro, Aβ-mediated activation of microglial cells can be blocked with antibodies directed against the ligand-binding domain of RAGE (Yan et al., Proc. Natl. Acad. Sci., USA, 94:5296-5301 (1997)). It has also been demonstrated that RAGE can serve as a focal point for fibril assembly (Deane et al., Nat. Med. 9:907-913 (2003)).

Also, in vivo inhibition of RAGE/ligand interactions using either sRAGE or an anti-RAGE antibody can reduce amyloid plaque formation in a mouse model of systemic amyloidosis (Yan et al., Nat. Med., 6:643-651 (2000)). Double transgenic mice that over-express human RAGE and human amyloid precursor protein (APP) with the Swedish and London mutations (mutant hAPP) in neurons develop learning defects and neuropathological abnormalities earlier than their single mutant hAPP transgenic counterparts. In contrast, double transgenic mice with diminished Aβ signaling capacity due to neurons expressing a dominant negative form of RAGE on the same mutant hAPP background, show a delayed onset of neuropathological and learning abnormalities compared to their single APP transgenic counterpart (Arancio et al., EMBO J., 23:4096-4105 (2004)).

In addition, inhibition of RAGE-amyloid interaction has been shown to decrease expression of cellular RAGE and cell stress markers (as well as NF-κB activation), and diminish amyloid deposition (Yan et al., Nat. Med., 6:643-651 (2000)) suggesting a role for RAGE-amyloid interaction in both perturbation of cellular properties in an environment enriched for amyloid (even at early stages) as well as in amyloid accumulation.

Thus, the RAGE fusion proteins of the present invention may also be used to reduce amyloidosis and to reduce amyloid plaques and cognitive dysfunction associated with Alzheimer's Disease (AD). As described above, sRAGE has been shown to reduce both amyloid plaque formation in the brain and subsequent increase in inflammatory markers in an animal model of AD. In studies using a mouse model of Alzheimer's Disease, it has been shown that RAGE antagonists can reverse the formation of plaques and the loss of cognition. In U.S. Patent Publication No. US 2005/0026811, small molecule RAGE antagonists were used to inhibit the progression of Aβ deposition and reduced the volume of preexisting plaques in Alzheimer's Disease mice (US 2005/0026811 at ¶¶581-586). Furthermore, treatment with such small molecule RAGE antagonists improved cognition in these Alzheimer's Disease mouse models (US 2005/0026811 at ¶¶587-590). Thus, in a mouse model of Alzheimer's Disease, those mice who had developed Aβ plaques and cognitive loss and were treated with small molecule RAGE antagonists exhibited a reduction in plaque volume and an improvement in cognitive performance as compared to those Alzheimer's Disease mice who were not treated with the small molecule RAGE antagonists, showing that the RAGE antagonist compounds may delay or slow loss of cognitive performance, or may improve cognitive performance of a subject suffering from dementia of Alzheimer's type.

Also, it had been shown in both cellular assays and in animal studies that RAGE mediates the transcytosis of circulating Aβ across the blood-brain barrier (BBB). Such increased transcytosis of Aβ results in neuronal oxidant stress and sustained reductions in cerebral blood flow. The effects of RAGE can be inhibited by a RAGE modulator (e.g., anti-RAGE antibody or sRAGE) (see e.g., Mackic et al., J. Clin. Invest., 102:734-743 (1998); see also Kumar et al., Neurosci., Program, p 141 (2000)). These finding were confirmed by additional studies (see e.g., U.S. Pat. No. 6,825,164 at col. 17, line 48 to col. 18, line 43; Deane et al., Nature Medicine, 9:907-913 (2003)). Reduced cerebral perfusion can promote ischemic lesions which can act synergistically with Aβ to exacerbate dementia. Also, insufficient cerebral blood flow may alter Aβ trafficking across the blood brain barrier thereby reducing Aβ clearance and promoting accumulation of Aβ in brain (see Girouard and Iadecola, J. Appl. Physiol., 100, 328-335 (2006) at page 332). Thus, the increase in cerebral blood flow promoted by RAGE antagonists may reduce the symptoms or delay onset of development of Alzheimer's Disease, or both. For example, RAGE antagonists may delay or slow loss of cognitive performance, or may improve cognitive performance of a subject suffering from dementia of Alzheimer's type, or both.

Alzheimer's Disease may be diagnosed by NINCDS and DSM criteria, Mini-Mental State Examination, and Clinical Dementia Rating within particular limits. One aspect of the present invention includes improving cognitive performance comprising administering a RAGE fusion protein of the present invention. Cognitive performance may be assessed with the cognitive subscale of the Alzheimer's Disease Assessment Scale (ADAS-cog), as is known in the art, which scores cognitive function on a 0 to 70 scale, with higher scores indicating greater cognitive impairment. Thus, a reduction in score demonstrates cognitive improvement. One aspect of the present invention includes administering to a subject a RAGE fusion protein of the present invention to reduce an ADAS-cog score of a subject. The subject may be a human be suffering from dementia of Alzheimer's type, mild to moderate Alzheimer's Diseases, or severe Alzheimer's Disease.

In addition, the progression of Alzheimer's Disease may also be assessed in a human through examination of four areas of function: General, Cognitive, Behavioral, and Activities of Daily Living. Such an assessment may be performed using a Clinician's Interview Based Impression of Change (CIBIC or CIBIC plus). One aspect of the present invention includes improvement in subject's function comprising administering a RAGE fusion protein of the present invention. In one embodiment, the subject's function is one or more of general, cognitive, behavioral, and activities of daily living.

In another embodiment, the RAGE fusion proteins of the present invention may be used to treat dementia associated with head trauma. In an embodiment, the cognitive performance of the subject is improved. In another embodiment, the cognitive performance of the subject is maintained. In another embodiment, the rate of loss of cognitive performance of the subject is slowed.

Also, RAGE fusion proteins of the present invention may be used to treat atherosclerosis and other cardiovascular disorders. Thus, it has been shown that ischemic heart disease is particularly high in patients with diabetes (Robertson, et al., Lab Invest., 18:538-551 (1968); Kannel et al, J. Am. Med. Assoc., 241:2035-2038 (1979); Kannel et al., Diab. Care, 2:120-126 (1979)). In addition, studies have shown that atherosclerosis in patients with diabetes is more accelerated and extensive than in patients not suffering from diabetes (see e.g. Waller et al., Am. J. Med., 69:498-506 (1980); Crall et al, Am. J. Med. 64:221-230 (1978); Hamby et al., Chest, 2:251-257 (1976); and Pyorala et al., Diab. Metab. Rev., 3:463-524 (1978)). Although the reasons for accelerated atherosclerosis in the setting of diabetes are many, it has been shown that reduction of AGEs can reduce plaque formation.

In another embodiment, the RAGE fusion proteins of the present invention may be used to treat cancer. In one embodiment, the cancer treated using the RAGE fusion proteins of the present invention comprises cancer cells that express RAGE. For example, cancers that may be treated with the RAGE fusion protein of the present invention include some lung cancers, some gliomas, some papillomas, and the like. Amphoterin is a high mobility group I nonhistone chromosomal DNA binding protein (Rauvala et al., J. Biol. Chem., 262:16625-16635 (1987); Parkikinen et al., J. Biol. Chem. 268:19726-19738 (1993)) which has been shown to interact with RAGE. It has been shown that amphoterin promotes neurite outgrowth, as well as serving as a surface for assembly of protease complexes in the fibrinolytic system (also known to contribute to cell mobility). In addition, a local tumor growth inhibitory effect of blocking RAGE has been observed in a primary tumor model (C6 glioma), the Lewis lung metastasis model (Taguchi et al., Nature 405:354-360 (2000)), and spontaneously arising papillomas in mice expressing the v-Ha-ras transgene (Leder et al., Proc. Natl. Acad. Sci., 87:9178-9182 (1990)).

In yet another embodiment, the RAGE fusion proteins of the present invention may be used to treat inflammation. In alternate embodiments, the RAGE fusion proteins of the present invention may be used to treat inflammation associated with inflammatory bowel disease, inflammation associated with rheumatoid arthritis, inflammation associated with psoriasis, inflammation associated with multiple sclerosis, inflammation associated with hypoxia, inflammation associated with stroke, inflammation associated with heart attack, inflammation associated with hemorrhagic shock, inflammation associated with sepsis, inflammation associated with organ transplantation, inflammation associated with impaired wound healing, or inflammation associated with rejection of self (e.g., autoimmune) or non-self (e.g., transplanted) cells, tissue, or organs.

For example, following thrombolytic treatment, inflammatory cells such as granulocytes infiltrate the ischemic tissue and produce oxygen radicals that can destroy more cells than were killed by the hypoxia. Inhibiting the receptor on the neutrophil responsible for the neutrophils being able to infiltrate the tissue with antibodies or other protein antagonists has been shown to ameliorate the response. Since RAGE is a ligand for this neutrophil receptor, a RAGE fusion protein containing a fragment of RAGE may act as a decoy and prevent the neutrophil from trafficking to the reperfused site and thus prevent further tissue destruction. The role of RAGE in prevention of inflammation may be indicated by studies showing that sRAGE inhibited neointimal expansion in a rat model of restenosis following arterial injury in both diabetic and normal rats, presumably by inhibiting endothelial, smooth muscle cell proliferation and macrophage activation via RAGE (Zhou et al., Circulation, 107:2238-2243 (2003)). In addition, sRAGE inhibited models of inflammation including delayed-type hypersensitivity, experimental autoimmune encephalitis and inflammatory bowel disease (Hofman et al., Cell, 97:889-901 (1999)).

In an embodiment, the RAGE fusion proteins of the present invention may be used to treat autoimmune based disorders. For example, in an embodiment, the RAGE fusion proteins of the present invention may be used to treat kidney failure. Thus, the RAGE fusion proteins of the present invention may be used to treat systemic lupus nephritis or inflammatory lupus nephritis. For example, the S100/calgranulins have been shown to comprise a family of closely related calcium-binding polypeptides characterized by two EF-hand regions linked by a connecting peptide (Schafer et al., TIBS, 21:134-140 (1996); Zimmer et al., Brain Res. Bull., 37:417-429 (1995); Rammes et al., J. Biol. Chem., 272:9496-9502 (1997); Lugering et al., Eur. J. Clin. Invest., 25:659-664 (1995)). Although they lack signal peptides, it has long been known that S100/calgranulins gain access to the extracellular space, especially at sites of chronic immune/inflammatory responses, as in cystic fibrosis and rheumatoid arthritis. RAGE is a receptor for many members of the S100/calgranulin family, mediating their proinflammatory effects on cells such as lymphocytes and mononuclear phagocytes. Also, studies on delayed-type hypersensitivity response, colitis in IL-10 null mice, collagen-induced arthritis, and experimental autoimmune encephalitis models suggest that RAGE-ligand interaction (presumably with S-100/calgranulins) has a proximal role in the inflammatory cascade.

In an embodiment, the RAGE fusion proteins of the present invention may be used to treat restenosis. In an embodiment, the subject is suffering from diabetes.

Type I diabetes is an autoimmune disorder that may be prevented or ameliorated by treatment with the RAGE fusion proteins of the present invention. For example, it has been shown that sRAGE may allow for the transfer of splenocytes from non-obese diabetic (NOD) mice to NOD-mice with severe combined immunodeficiency (NOD-scid mice). NOD-scid mice do not display diabetes spontaneously, but require the presence of immunocytes capable of destroying islet cells such that diabetes is then induced. It was found that NOD-scid recipients treated with sRAGE displayed reduced onset of diabetes induced by splenocytes transferred from a diabetic (NOD) mouse as compared to NOD-scid recipients not treated with sRAGE (U.S. Patent Publication 2002/0122799). As stated in US 2002/0122799, the experimental results using sRAGE in this model are relevant to human disease such as clinical settings in which future immune therapies and islet transplantation may occur.

Thus, in an embodiment, a RAGE fusion protein of the present invention may be used to treat inflammation associated with transplantation of at least one of an organ, a tissue, or a plurality of cells from a first site to a second site. The first and second sites may be in different subjects, or in the same subject. In alternate embodiments, the transplanted cells, tissue or organ comprise cells of a pancreas, skin, liver, kidney, heart, lung, bone marrow, blood, bone, muscle, endothelial cells, artery, vein, cartilage, thyroid, nervous system, or stem cells. For example, administration of the RAGE fusion proteins of the present invention may be used to facilitate transplantation of islet cells from a first non-diabetic subject to a second diabetic subject.

In another embodiment, the present invention may provide a method of treating osteoporosis by administering to a subject an amount of a RAGE fusion protein of the present invention. (Zhou et al., J. Exp. Med., 203:1067-1080 (2006)). In an embodiment, the method of treating osteoporosis may further comprise the step of increasing bone density of the subject or reducing the rate of decrease in bone density of a subject.

Thus, in various selected embodiments, the present invention may provide a method for inhibiting the interaction of an AGE with RAGE in a subject by administering to the subject an amount of a RAGE fusion protein of the present invention sufficient to inhibit this interaction. The subject treated using the RAGE fusion proteins of the present invention may be an animal. In an embodiment, the subject is a human. The subject may be suffering from an AGE-related disease such as diabetes, diabetic complications such as nephropathy, neuropathy, retinopathy, foot ulcer, amyloidoses, or renal failure, and inflammation. Or, the subject may be an individual with Alzheimer's disease. In an alternative embodiment, the subject may be an individual with cancer. In yet other embodiments, the subject may be suffering from systemic lupus erythmetosis or inflammatory lupus nephritis. Other diseases may be mediated by RAGE and thus, may be treated using the RAGE fusion proteins of the present invention. Thus, in additional alternative embodiments of the present invention, the RAGE fusion proteins may be used for treatment of Crohn's disease, arthritis, vasculitis, nephropathies, retinopathies, and neuropathies in human or animal subjects. In other embodiments, inflammation involving both autoimmune responses (e.g., rejection of self) and non-autoimmune responses (e.g., rejection of non-self) may be mediated by RAGE and thus, may be treated using the RAGE fusion proteins of the present invention.

As described above, the present invention relates to a RAGE fusion protein of any of the previous embodiments for inhibiting the interaction of an AGE with RAGE in a subject by administering to the subject an amount of a RAGE fusion protein of the present invention sufficient to inhibit this interaction. In an embodiment, the RAGE fusion protein may be used in medicine for treatment of a RAGE-mediated disorder or disease.

Thus, embodiments of the present invention also relate to the use of a RAGE fusion protein for the treatment of any one or several of the following conditions: diabetes or diabetic late complications, osteoporosis, amyloidosis, Alzheimer's disease, cancer, kidney failure, or inflammation associated with autoimmunity, inflammatory bowel disease, rheumatoid arthritis, psoriasis, multiple sclerosis, hypoxia, stroke, heart attack, hemorrhagic shock, sepsis, organ transplantation, or impaired wound healing.

The present invention also relates to the use of a RAGE fusion protein in the manufacture of a medicament for the treatment of any one or several of diabetes or diabetic late complications, osteoporosis, amyloidosis, Alzheimer's disease, cancer, kidney failure, or inflammation associated with autoimmunity, inflammatory bowel disease, rheumatoid arthritis, psoriasis, multiple sclerosis, hypoxia, stroke, heart attack, hemorrhagic shock, sepsis, organ transplantation, or impaired wound healing.

A therapeutically effective amount may comprise an amount which is capable of inhibiting and/or preventing the interaction of RAGE with an AGE or other types of endogenous RAGE ligands in a subject. Accordingly, the amount will vary with the subject being treated. Administration of the compound may be hourly, daily, weekly, monthly, yearly, or as a single event. In various alternative embodiments, the therapeutically effective amount of the RAGE fusion protein may range from about 1 ng/kg body weight to about 100 mg/kg body weight, or from about 10 μg/kg body weight to about 50 mg/kg body weight, or from about 100 μg/kg body weight to about 20 mg/kg body weight. The actual effective amount may be established by dose/response assays using methods standard in the art (Johnson et al., Diabetes. 42: 1179, (1993)). Thus, as is known to those in the art, the effective amount may depend on bioavailability, bioactivity, and biodegradability of the compound.

### Compositions

The present invention may comprise a composition comprising a RAGE fusion protein of any one of the previous embodiments and a pharmaceutically acceptable carrier.

Pharmaceutically acceptable carriers may comprise any of the standard pharmaceutically accepted carriers known in the art. In one embodiment, the pharmaceutical carrier may be a liquid and the RAGE fusion protein or nucleic acid construct formulation may be in the form of a solution. In another embodiment, the pharmaceutically acceptable carrier may be a solid in the form of a powder, a lyophilized powder, or a tablet. Or, the pharmaceutical carrier may be a gel, suppository, or cream. In alternate embodiments, the carrier may comprise a liposome, a microcapsule, a polymer encapsulated cell, or a virus. Thus, the term pharmaceutically acceptable carrier encompasses, but is not limited to, any of the standard pharmaceutically accepted carriers, such as water, alcohols, phosphate buffered saline solution, sugars (e.g., sucrose or mannitol), oils or emulsions such as oil/water emulsions or a triglyceride emulsion, various types of wetting agents, tablets, coated tablets and capsules.

In certain embodiments, the RAGE fusion proteins may be present in a neutral form (including zwitter ionic forms) or as a positively or negatively-charged species. In some embodiments, the RAGE fusion proteins may be complexed with a counterion to form a pharmaceutically acceptable salt.

The terms “pharmaceutically acceptable salt” refer to a complex comprising one or more RAGE fusion proteins and one or more counterions, where the counterions are derived from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, cobalt, nickel, molybdenum, vanadium, manganese, chromium, selenium, tin, copper, ferric, ferrous, lithium, magnesium, manganic salts, manganous, potassium, rubidium, sodium, and zinc, and in their usual valences.

Pharmaceutically acceptable acid addition salts of the RAGE fusion proteins of the present invention can be prepared from the following acids, including, without limitation formic, acetic, acetamidobenzoic, adipic, ascorbic, boric, propionic, benzoic, camphoric, carbonic, cyclamic, dehydrocholic, malonic, edetic, ethylsulfuric, fendizoic, metaphosphoric, succinic, glycolic, gluconic, lactic, malic, tartaric, tannic, citric, nitric, ascorbic, glucuronic, maleic, folic, fumaric, propionic, pyruvic, aspartic, glutamic, benzoic, hydrochloric, hydrobromic, hydroiodic, lysine, isocitric, trifluoroacetic, pamoic, propionic, anthranilic, mesylic, orotic, oxalic, oxalacetic, oleic, stearic, salicylic, aminosalicylic, silicate, p-hydroxybenzoic, nicotinic, phenylacetic, mandelic, embonic, sulfonic, methanesulfonic, phosphoric, phosphonic, ethanesulfonic, ethanedisulfonic, ammonium, benzenesulfonic, pantothenic, naphthalenesulfonic, toluenesulfonic, 2-hydroxyethanesulfonic, sulfanilic, sulfuric, nitric, nitrous, sulfuric acid monomethyl ester, cyclohexylaminosulfonic, β-hydroxybutyric, glycine, glycylglycine, glutamic, cacodylate, diaminohexanoic, camphorsulfonic, gluconic, thiocyanic, oxoglutaric, pyridoxal 5-phosphate, chlorophenoxyacetic, undecanoic, N-acetyl-L-aspartic, galactaric and galacturonic acids.

Pharmaceutically acceptable organic bases include trimethylamine, diethylamine, N,N′-dibenzylethylenediamine, chloroprocaine, choline, dibenzylamine, diethanolamine, ethylenediamine, meglumine (N-methylglucamine), procaine, cyclic amines, quaternary ammonium cations, arginine, betaine, caffeine, clemizole, 2-ethylaminoethanol, 2-diethylaminoethanol, 2-dimethylaminoethanol, ethanediamine, butylamine, ethanolamine, ethylenediamine, N-ethylmorpholine, N-ethylpiperidine, ethylglucamine, glucamine, glucosamine, histidine, hydrabamine, imidazole, isopropylamine, methylglucamine, morpholine, piperazine, pyridine, pyridoxine, neodymium, piperidine, polyamine resins, procaine, purines, theobromine, triethylamine, tripropylamine, triethanolamine, tromethamine, methylamine, taurine, cholate, 6-amino-2-methyl-2-heptanol, 2-amino-2-methyl-1,3-propanediol, 2-amino-2-methyl-1-propanol, aliphatic mono- and dicarboxylic acids, phenyl-substituted alkanoic acids, hydroxy alkanoic acids, aromatic acids, aliphatic and aromatic sulfonic acids, strontium, tricine, hydrazine, phenylcyclohexylamine, 2-(N-morpholino)ethanesulfonic acid, bis(2-hydroxyethyl)amino-tris(hydroxymethyl)methane, N-(2-acetamido)-2-aminoethanesulfonic acid, 1,4-piperazinediethanesulfonic acid, 3-morpholino-2-hydroxypropanesulfonic acid, 1,3-bis[tris(hydroxymethyl)methylamino]propane, 4-morpholinepropanesulfonic acid, 4-(2-hydroxyethyl)piperazine-1-ethanesulfonic acid, 2-[(2-hydroxy-1,1-bis(hydroxymethyl)ethyl)amino]ethanesulfonic acid, N,N-bis(2-hydroxyethyl)-2-aminoethanesulfonic acid, 4-(N-morpholino)butanesulfonic acid, 3-(N,N-bis[2-hydroxyethyl]amino)-2-hydroxypropanesulfonic acid, 2-hydroxy-3-[tris(hydroxymethyl)methylamino]-1-propanesulfonic acid, 4-(2-hydroxyethyl)piperazine-1-(2-hydroxypropanesulfonic acid), piperazine-1,4-bis(2-hydroxypropanesulfonic acid)dihydrate, 4-(2-hydroxyethyl)-1-piperazinepropanesulfonic acid, N,N-bis(2-hydroxyethyl)glycine, N-(2-hydroxyethyl)piperazine-N′-(4-butanesulfonic acid), N-[tris(hydroxymethyl)methyl]-3-aminopropanesulfonic acid, N-tris(Hydroxymethyl)methyl-4-aminobutanesulfonic acid, N-(1,1-dimethyl-2-hydroxyethyl)-3-amino-2-hydroxypropanesulfonic acid, 2-(cyclohexylamino)ethanesulfonic acid, 3-(cyclohexylamino)-2-hydroxy-1-propanesulfonic acid, 3-(cyclohexylamino)-1-propanesulfonic acid, N-(2-acetamido)iminodiacetic acid, 4-(cyclohexylamino)-1-butanesulfonic acid, N-[tris(hydroxymethyl)methyl]glycine, 2-amino-2-(hydroxymethyl)-1,3-propanediol, and trometamol.

In a further aspect of the present invention, the RAGE fusion proteins of the invention may be utilized in adjuvant therapeutic or combination therapeutic treatments with other known therapeutic agents. The following is a non-exhaustive listing of adjuvants and additional therapeutic agents which may be utilized in combination with the RAGE fusion protein modulators of the present invention:

Pharmacologic Classifications of Anticancer Agents:
1. Alkylating agents: Cyclophosphamide, nitrosoureas, carboplatin, cisplatin, procarbazine
2. Antibiotics: Bleomycin, Daunorubicin, Doxorubicin
3. Antimetabolites: Methotrexate, Cytarabine, Fluorouracil, Azathioprine, 6-Mercaptopurine, and cytotoxic cancer chemotherapeutic agents
4. Plant alkaloids: Vinblastine, Vincristine, Etoposide, Paclitaxel,
5. Hormones: Tamoxifen, Octreotide acetate, Finasteride, Flutamide
6. Biologic response modifiers: Interferons, Interleukins

Pharmacologic Classifications of Treatment for Rheumatoid Arthritis
1. Analgesics: Aspirin
2. NSAIDs (Nonsteroidal anti-inflammatory drugs): Ibuprofen, Naproxen, Diclofenac
   3. DMARDs (Disease-Modifying Antirheumatic drugs): Methotrexate, gold preparations, hydroxychloroquine, sulfasalazine
4. Biologic Response Modifiers, DMARDs: Etanercept, Infliximab Glucocorticoids, such as beclomethasone, methylprednisolone, betamethasone, prednisone, dexamethasone, and hydrocortisone

Pharmacologic Classifications of Treatment for Diabetes Mellitus
1. Sulfonylureas: Tolbutamide, Tolazamide, Glyburide, Glipizide
2. Biguanides: Metformin
3. Miscellaneous oral agents: Acarbose, Troglitazone
4. Insulin

Pharmacologic Classifications of Treatment for Alzheimer's Disease
1. Cholinesterase Inhibitor: Tacrine, Donepezil
2. Antipsychotics: Haloperidol, Thioridazine
3. Antidepressants: Desipramine, Fluoxetine, Trazodone, Paroxetine
4. Anticonvulsants: Carbamazepine, Valproic acid

In an embodiment, the compositions of the present invention may comprise an amount of a RAGE fusion protein in combination with a single or multiple additional therapeutic agents. In addition to the agents heretofore described, the following therapeutic agents may be used in combination with the RAGE fusion proteins of the present invention: immunosuppressants, such as cyclosporin, tacrolimus, rapamycin and other FK-506 type immunosuppressants.

In one embodiment, the present invention may therefore provide a method of treating RAGE mediated diseases, the method comprising administering to a subject, an amount of a fusion protein of the present invention in combination with therapeutic agents selected from the group consisting of alkylating agents, antimetabolites, plant alkaloids, antibiotics, hormones, biologic response modifiers, analgesics, NSAIDs, DMARDs, biologic response modifiers (e.g., glucocorticoids), sulfonylureas, biguanides, insulin, cholinesterase inhibitors, antipsychotics, antidepressants, anticonvulsants, and immunosuppressants, such as cyclosporin, tacrolimus, rapamycin and other FK-506 type immunosuppressants. In a further embodiment, the present invention provides the pharmaceutical composition of the invention as described above, further comprising one or more therapeutic agents selected from the group consisting of alkylating agents, antimetabolites, plant alkaloids, antibiotics, hormones, biologic response modifiers, analgesics, NSAIDs, DMARDs, biologic response modifiers (e.g., glucocorticoids), sulfonylureas, biguanides, insulin, cholinesterase inhibitors, antipsychotics, antidepressants, anticonvulsants, and immunosuppressants, such as cyclosporin, tacrolimus, rapamycin and other FK-506 type immunosuppressants.

Where the present invention relates to a method of treating a RAGE mediated diseases comprising administering to a subject an amount of a RAGE fusion protein in combination with an additional therapeutic agent, the order of administration of the RAGE fusion protein and the additional active agent may be simultaneous, subsequent, or sequential administration.

### Lyophilized Formulations

In other embodiments, the present invention also provides formulations comprising a RAGE fusion protein. Embodiments of the formulations may comprise a solid mixture of a lyoprotectant, a RAGE fusion protein, and buffer. The solid may be prepared by lyophilization of an aqueous solution comprising a lyoprotectant, a RAGE fusion protein, and buffer.

The RAGE fusion protein formulation may comprise a stable therapeutic agent that is formulated for use in a clinic or as a prescription medicine. For example, in certain embodiments, the RAGE fusion protein formulation may exhibits less than 10%, or less than 5%, or less than 3% decomposition after one week at 40 degrees Centigrade.

Also, the RAGE fusion protein formulation may be stable upon reconstitution in a diluent. In certain embodiments, less than about 10%, or about 5%, or about 4%, or about 3%, or about 2%, or about 1% of the RAGE fusion protein is present as an aggregate in the RAGE fusion protein formulation.

Methods of administration of either nucleic acid based or protein based compositions of the invention, particularly RAGE fusion proteins or expression constructs as described herein, can be by any of a number of methods well known in the art. These methods include local or systemic administration and may be administered intracerebroventricularly, intrathecally, intradermally, intramuscularly, intraperitoneally, intravenously, intra-arterially, subcutaneously, rectally, orally, buccally, sublingually, intranasally, or as an aerosol. In addition, it may be desirable to introduce either nucleic acid based or protein based compositions of the invention into the central nervous system by any suitable route, including intraventricular, intrathecal, intracerebroventricular, and epidural administration via injection or via infusion using a pump. For example, intraventricular injection may be facilitated by an intraventricular catheter attached to a reservoir, such as an Ommaya reservoir. Infusion using a pump may be facilitated, for example, by an implantable pump attached to a catheter. In another embodiment, intracerebroventricular injection may be facilitated by an implantable pump attached to a catheter.

These and other implants and prosthetics can be treated with the subject fusion proteins or with an expression construct containing a nucleic acid expressing a subject fusion protein. In this way, the subject fusion proteins can be administered directly to the specific affected tissue.

Methods of introduction may also be provided by rechargeable or biodegradable devices. Furthermore, it is contemplated that administration may occur by coating a device, implant, stent, or prosthetic.

As described in more detail herein, RAGE has been implicated in the pathogenesis of a variety of disease states. Thus, the RAGE fusion protein formulations of the present invention may be used to treat a variety of RAGE-mediated disorders.

In certain embodiments, a RAGE fusion protein formulation of the present invention may be used to treat a symptom of diabetes or a symptom of diabetic late complications. For example, the symptom of diabetes or diabetic late complications comprises at least one of diabetic nephropathy, diabetic retinopathy, a diabetic foot ulcer, a cardiovascular complication, or diabetic neuropathy.

In other embodiments, a RAGE fusion protein formulation of the present invention may be used to treat at least one of amyloidosis, Alzheimer's disease, cancer, kidney failure, or inflammation associated with autoimmunity, inflammatory bowel disease, rheumatoid arthritis, psoriasis, multiple sclerosis, hypoxia, stroke, heart attack, hemorrhagic shock, sepsis, organ transplantation, or impaired wound healing.

Or, the RAGE fusion protein formulation may be used to treat osteoporosis. For example, in certain embodiments, administration of a RAGE fusion protein of the present invention increases bone density of subject or reduces the rate of a decrease in bone density of a subject.

In some embodiments, the autoimmunity treated using the RAGE fusion protein formulations of the present invention may comprise rejection of at least one of skin cells, pancreatic cells, nerve cells, muscle cells, endothelial cells, heart cells, liver cells, kidney cells, a heart, bone marrow cells, bone, blood cells, artery cells, vein cells, cartilage cells, thyroid, cells, or stem cells. Or, the RAGE fusion protein formulation may be used to treat kidney failure.

In certain embodiments, the RAGE fusion protein formulation may used to treat inflammation and/or rejection associated with transplantation of at least one of an organ, a tissue, or a plurality of cells from a first site to a second site. The first and second sites may either be in different subjects, or in the same subject. Transplantation of a variety of different cell types may be improved using the RAGE fusion protein formulations of the present invention. For example, the transplanted cells, tissue, or organ may comprise a cell, tissue or organ of a pancreas, skin, liver, kidney, heart, bone marrow, blood, bone, muscle, artery, vein, cartilage, thyroid, nervous system, or stem cells.

A variety of lyoprotectants may be used in the lyophilized RAGE fusion protein formulations of the present invention. In some embodiments, the lyoprotectant may comprise a non-reducing sugar. For example, the non-reducing sugar may comprise sucrose, mannitol, or trehalose. Also, a variety of buffers may be used in the lyophilized RAGE fusion protein formulation. In certain embodiments, the buffer may comprise histidine.

The solid lyophilized RAGE fusion protein formulation may comprise additional components. In certain embodiments, the RAGE fusion protein formulation may further comprise at least one of a surfactant, a chelating agent or a bulking agent.

In one embodiment, the present invention comprises a reconstituted formulation comprising a lyophylized RAGE fusion protein reconstituted in a diluent, wherein the RAGE fusion protein concentration in the reconstituted formulation is within the range from about 1 mg/mL to about 400 mg/mL. Or, other concentrations of the RAGE fusion protein may be used as described herein.

In other embodiments, the present invention may also comprise methods for preparing stable reconstituted formulation of a RAGE fusion protein. The reconstituted formulation may comprise a concentration that is suitable for direct use (e.g., direct administration to a subject) or that may be further diluted and/or mixed with a delivery agent.

In certain embodiments, the method may comprise reconstituting a lyophilized mixture of the RAGE fusion protein and a lyoprotectant in a diluent such that the RAGE fusion protein concentration in the reconstituted formulation is in a range from about 1 mg/mL to about 400 mg/mL. Or, other concentrations as described herein may be used as described herein.

A variety of lyoprotectants may be used in the reconstituted RAGE fusion protein formulations of the present invention. In some embodiments, the lyoprotectant may comprise a non-reducing sugar. For example, the non-reducing sugar may comprise sucrose, mannitol, or trehalose. Also, a variety of buffers may be used in the lyophilized RAGE fusion protein formulation. In certain embodiments, the buffer may comprise histidine.

The reconstituted RAGE fusion protein formulation may comprise additional components. In certain embodiments, the RAGE fusion protein formulation may further comprise at least one of a surfactant, a chelating agent or a bulking agent.

A variety of diluents suitable for pharmaceuticals may be used to reconstitute the lyophilized RAGE fusion protein. In an embodiment, the lyophilized RAGE fusion protein is sterile. Also in an embodiment, the diluent is sterile. In one embodiment, the diluent may comprise water for injection (WFI). Also, in certain embodiments, the amount of diluent added is based on the therapeutic dosage and the pharmacokinetic profile of the RAGE fusion protein, as well as the biocompatibility of the formulation and carrier being administered. In an embodiment, the reconstituted formulation is isotonic.

The reconstituted RAGE fusion protein formulation may be suitable for administration by various routes and as is required for treatment of the RAGE-mediated disorder of interest. In certain embodiments, the reconstituted RAGE fusion protein formulation is suitable for at least one of intravenous, intraperitoneal, subcutaneous, intraventricular, intrathecal, intracerebroventricular, or epidural administration of the formulation to a subject.

In yet other embodiments, the present invention may comprise articles of manufacture that include RAGE fusion proteins. For example, in certain embodiments, the article of manufacture may comprise a container which holds a lyophylized RAGE fusion protein, and instructions for reconstituting the lyophilized formulation with a diluent. In certain embodiments, the articles of manufacture may comprise a container which holds a formulation comprising a lyophilized mixture of a lyoprotectant, a RAGE fusion protein, and buffer. The article of manufacture may also comprise instructions for reconstituting the lyophilized formulation with a diluent.

A variety of lyoprotectants may be used in the articles of manufacture of the present invention. In some embodiments, the lyoprotectant may comprise a non-reducing sugar. For example, the non-reducing sugar may comprise sucrose, mannitol, or trehalose. Also, a variety of buffers may be used in the lyophilized RAGE fusion protein formulation. In certain embodiments, the buffer may comprise histidine.

The RAGE fusion protein formulation of the articles of manufacture of the present invention may comprise additional components. In certain embodiments, the lyophilized RAGE fusion protein formulation may further comprise at least one of a surfactant, a chelating agent or a bulking agent.

A variety of diluents suitable for pharmaceuticals may be provided for reconstituting the lyophilized RAGE fusion protein. In an embodiment, the lyophilized formulation is sterile. Alternatively or additionally, the diluent may be sterile. In one embodiment, the diluent may comprise water for injection (WFI). Thus, the article of manufacture may further comprise a second container which holds a diluent for reconstituting the lyophilized formulation, wherein the diluent is water for injection (WFI). In an embodiment, the reconstituted formulation is isotonic.

Also, in certain embodiments, the amount of diluent added is based on the therapeutic dosage and the pharmacokinetic profile of the RAGE fusion protein, as well as the biocompatibility of the formulation and carrier being administered. In alternate embodiment, the instructions are for reconstituting the lyophilized formulation so as to have the concentrations as described herein. For example, in certain embodiments, the instructions are for reconstituting the lyophilized formulation such that the RAGE fusion protein concentration in the reconstituted formulation is within the range from about 40 mg/mL to about 100 mg/mL.

Also, in certain embodiments, when reconstituted according to the instructions provided, the reconstituted RAGE fusion protein formulation may be suitable for administration by various routes and as is required for treatment of the RAGE-mediated disorder of interest. In certain embodiments, the reconstituted RAGE fusion protein formulation is suitable for at least one of intravenous, intraperitoneal, subcutaneous, intraventricular, intrathecal, intracerebroventricular, or epidural administration of the formulation to the subject.

In certain embodiments of the formulations, articles of manufacture, and methods of making formulations comprising a RAGE fusion protein, the RAGE fusion protein concentration in the reconstituted formulation may be at least 10 mg/mL, or at least 20 mg/mL, or at least 50 mg/mL. In alternate embodiments, the RAGE fusion protein concentration in the reconstituted formulation may be at least 100 mg/mL, or 200 mg/mL, or 400 mg/mL. For example, in alternate embodiments, the RAGE fusion protein concentration in the reconstituted formulation is at least about 0.5 to 400 mg/mL, or about 1 to 200 mg/mL, 40 to 400 mg/mL, 50 to 400 mg/mL, 40 to 100 mg/mL, 50 to 100 mg/mL, or about 40-50 mg/mL.

Any of the embodiments described herein may be used as the RAGE fusion protein in the formulations of the present invention. Thus, the embodiments of RAGE fusion proteins as described herein may be used for each of the lyophilized formulations, reconstituted lyophilized formulations, or the methods of making the lyophilized formulations or reconstituted lyophilized formulations, or the articles of manufacture comprising either the lyophilized formulations or the reconstituted lyophilized formulations of the present invention.

### Preparation of Lyophilized Formulations

In another embodiment, the present invention provides a pre-lyophilized formulation, a lyophilized formulation, a reconstituted formulation, and methods for preparation thereof.

After preparation of a RAGE fusion protein of interest as described above, a “pre-lyophilized formulation” may be produced. The amount of RAGE fusion protein present in the pre-lyophilized formulation may be determined taking into account the desired dose volumes, mode(s) of administration etc. In an embodiment, the amount of fusion protein in the pre-lyophilized formulation may be greater than 1 mg/mL. Also in certain embodiments, the amount of fusion protein in the pre-lyophilized formulation may be less than about 5 mg/mL, 10 mg/mL, 50 mg/mL, 100 mg/mL, or 200 mg/mL.

In a further embodiment, the pre-lyophilized formulation may be a pH-buffered solution at a pH from about 4-8. In another embodiment, the pre-lyophilized formulation may be a pH-buffered solution at a pH from about 5-7. Exemplary buffers include histidine, phosphate, Tris, citrate, succinate and other organic acids as described herein. The buffer concentration may be from about 1 mM to about 100 mM, or less than about 50 mM, or from about 2 mM to about 50 mM, or less than about 15 mM, or from about 3 mM to about 15 mM depending, for example, on the buffer and the desired isotonicity of the formulation (e.g. of the reconstituted formulation). In an embodiment, the buffer is histidine.

The lyoprotectant may be added to the pre-lyophilized formulation. In an embodiment, the lyoprotectant comprises a sugar. In another embodiment, the lyoprotectant comprises a non-reducing sugar. In another embodiment, the lyoprotectant comprises the non-reducing sugar sucrose. Or, the non-reducing sugar may comprise mannitol. Or, the non-reducing sugar may comprise trehalose. The amount of lyoprotectant in the pre-lyophilized formulation is generally such that upon reconstitution, the resulting formulation will be isotonic. However, a hypertonic reconstituted formulation may also be suitable, for example in formulations for peripheral parenteral administration. In addition, the amount of lyoprotectant should not be so low such that an unacceptable amount of degradation and/or aggregation of the protein occurs upon lyophilization. In alternate embodiments, an unacceptable amount of aggregation may be where 20%, or 10%, or 5% or more of the RAGE fusion protein is present as an aggregate in a formulation. An exemplary range of lyoprotectant concentration in the pre-lyophilized formulation may be less than about 400 mM. In another embodiment, the range of lyoprotectant concentration in the pre-lyophilized formulation is less than about 100 mM. In alternate embodiments, the range of lyoprotectant concentration in the pre-lyophilized formulation may therefore range from about 0.5 mM to 400 mM, or from about 2 mM to 200 mM, or from about 30 mM to about 150 mM, or from about 60-65 mM. Or, ranges within these ranges may be used. Also, in some embodiments, the lyoprotectant is added in an amount to render the reconstituted formulation isotonic.

The ratio of RAGE fusion protein to lyoprotectant in the pre-lyophilized formulation is selected for each RAGE fusion protein and lyoprotectant combination. In an embodiment of an isotonic reconstituted formulation with a high RAGE fusion protein concentration (e.g., greater than or equal to about 50 mg/mL), the molar ratio of lyoprotectant to RAGE fusion protein may be from about 50 to about 1500 moles lyoprotectant to 1 mole RAGE fusion protein. In another embodiment, the molar ratio of lyoprotectant to RAGE fusion protein may be from about 150 to about 1000 moles of lyoprotectant to 1 mole fusion protein. In another embodiment, the molar ratio of lyoprotectant to RAGE fusion protein may be from about 150 to about 300 moles of lyoprotectant to 1 mole RAGE fusion protein. Or, ranges within these ranges may be used. For example, these ranges may be suitable where the lyoprotectant is a non-reducing sugar, such as sucrose, trehalose or mannitol.

In another embodiment of the invention, a surfactant may be added to the pre-lyophilized formulation. Alternatively, or in addition, the surfactant may be added to the lyophilized formulation and/or the reconstituted formulation. Exemplary surfactants include nonionic surfactants such as polysorbates (e.g. polysorbates 20 or 80) (Tween 20™ or Tween 80™); poloxamers (e.g. poloxamer 188). The amount of surfactant added is such that it reduces aggregation of the reconstituted protein and minimizes the formation of particulates after reconstitution. For example, the surfactant may be present in the pre-lyophilized formulation in an amount from about 0.001% to 0.5%. For example, in an embodiment where the surfactant comprises polysorbate 80, the surfactant may be present in the pre-lyophilized formulation in an amount from about 0.005% to 0.05%, or about 0.008% to 0.012%, or at about 0.01%. Alternatively, the surfactant may be present in the formulation so as to comprise a final concentration ranging from 0.001 mg/mL to about 100 mg/mL, or about 0.01 mg/mL to about 10 mg/mL. Or, ranges within these ranges may be used.

In certain embodiments of the invention, a mixture of the lyoprotectant (such as sucrose or histidine) and a bulking agent (e.g. mannitol or glycine) may be used in the preparation of the pre-lyophilization formulation. The bulking agent may allow for the production of a uniform lyophilized cake without excessive pockets therein etc.

Other pharmaceutically acceptable carriers, excipients or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include additional buffering agents; preservatives; co-solvents; antioxidants including ascorbic acid and methionine; chelating agents such as EDTA; metal complexes (e.g. Zn-protein complexes); biodegradable polymers such as polyesters; and/or salt-forming counter ions such as sodium.

The RAGE fusion protein formulations of the present invention may also contain additional proteins as necessary for the particular indication being treated. The additional proteins may be selected such that the proteins each have complementary activities that do not adversely affect each other or the RAGE fusion protein. Such proteins are suitably present in combination in amounts that are effective for the purpose intended.

The RAGE fusion protein formulations of the present invention may be sterile for in vivo administration. This may be accomplished by filtration through sterile filtration membranes, prior to, or following, lyophilization and reconstitution.

After the RAGE fusion protein, lyoprotectant and other optional components are mixed together, the formulation may be lyophilized. Many different freeze-dryers are available for this purpose such as Hull50™ (Hull, USA) or GT20™ (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −50 to 25° C. (provided the product remains frozen during primary drying) at a suitable pressure, ranging typically from about 50 to 250 mTorr. In an embodiment, the pressure is about 100 mTorr and the sample may be lyophilized between about −30 and 25° C. The formulation, size and type of the container holding the sample (e.g., glass vial) and the volume of liquid may dictate the time required for drying, which can range from a few hours to several days (e.g., 40-60 hrs). Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 2, 3, 5, 10, 20, 50, 100, or 250 cc vial. In an embodiment, the container is any container suitable to prepare a reconstituted formulation having a volume of less than or equal to 100 mL.

As a general proposition, lyophilization will result in a lyophilized formulation in which the moisture content thereof is less than about 5%. In an embodiment, the moisture content of the lyophilized formulation is less than about 3%. In another embodiment, the moisture content of the lyophilized formulation is less than about 1%.

Reconstitution of the Lyophilized Formulation

At the desired stage, typically when it is time to administer the RAGE fusion protein to a patient or subject, the lyophilized formulation may be reconstituted with a diluent such that the RAGE fusion protein concentration in the reconstituted formulation is about greater than 10 mg/mL, or greater than 20 mg/ml, or greater than 50 mg/mL, or about 30-50 mg/mL, or about 50 mg/mL. In alternate embodiments, the RAGE fusion protein concentration in the reconstituted formulation may be at least 100 mg/mL, or 200 mg/mL, or 400 mg/mL. For example, in alternate embodiments, the RAGE fusion protein concentration in the reconstituted formulation may be in the range of from about 1 mg/mL to about 600 mg/mL, or from about 1 mg/mL to about 500 mg/mL, or from about 1 mg/mL to about 400 mg/mL, or from about 1 mg/mL to about 200 mg/mL, or from about 10 mg/mL to about 400 mg/mL, or from about 10 mg/mL to about 200 mg/mL, or from about 40 mg/mL to about 400 mg/mL, or from about 40 mg/mL to about 200 mg/mL, or from about 50 mg/mL to about 400 mg/mL, or from about 50 mg/mL to about 200 mg/mL. In other embodiments, the RAGE fusion protein concentration in the reconstituted formulation is from about 40 mg/mL to about 100 mg/mL, or about 50 mg/mL to about 100 mg/mL, or about 40 mg/mL to about 50 mg/mL. Such RAGE fusion protein concentrations in the reconstituted formulation are considered to be particularly useful where subcutaneous delivery of the reconstituted formulation is intended. However, for other routes of administration, such as intravenous administration, lower concentrations of the protein in the reconstituted formulation may be desired (for example from about 5-50 mg/mL, or from about 10-40 mg/mL RAGE fusion protein in the reconstituted formulation). Thus, in some embodiments, the concentration of fusion protein in the reconstituted formulation may the same or less than 2 times the concentration of the fusion protein in the pre-lyophilized formulation.

In certain embodiments, the fusion protein concentration in the reconstituted formulation is significantly higher than that in the pre-lyophilized formulation. For example, the fusion protein concentration in the reconstituted formulation may, in certain embodiments, be about 2-40, or 2-10, or 3-8 times that of the pre-lyophilized formulation. In an embodiment, the RAGE fusion protein concentration in the reconstituted formulation may be about 3-6 times that of the pre-lyophilized formulation. In another embodiment where the concentration of RAGE fusion protein in the pre-lyophilized formulation is about 15 mg/mL, the concentration of the RAGE fusion protein in the reconstituted formulation is greater than or equal to about 50 mg/mL (e.g., at least three fold or at least four fold greater).

The delivery of a high protein concentration is often advantageous or required for subcutaneous administration due to the volume limitations (less than or equal to 1.5 mL) and dosing requirements (greater than or equal to 100 mg). However, protein concentrations (greater than or equal to 50 mg/mL) may be difficult to achieve in the manufacturing process since at high concentrations, a protein may have a tendency to aggregate during processing and become difficult to manipulate (e.g. pump) and sterile filter. Alternatively, the lyophilization process may provide a method to allow concentration of a protein. For example, a RAGE fusion protein may be filled into vials at a volume (Vf) and then lyophilized. The lyophilized RAGE fusion protein is then reconstituted with a smaller volume (Vr) of water or preservative (e.g. BWFI) than the original volume (e.g. Vr=0.25 Vf) resulting in a higher RAGE fusion protein concentration in the reconstituted solution. This process also results in the concentration of the buffers and excipients. For subcutaneous administration, the solution is desirably isotonic.

Reconstitution generally takes place at a temperature of about 25° C. to ensure complete hydration, although other temperatures may be employed as desired. The time required for reconstitution may depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. In an embodiment, the diluent provides a reconstituted formulation suitable for injection. In another embodiment, where the diluent provides a reconstituted formulation suitable for injection, the diluent comprises water for injection (WFI). The diluent optionally contains a preservative. The amount of preservative employed may be determined by assessing different preservative concentrations for compatibility with the protein and preservative efficacy testing.

Administration of the Reconstituted Formulation

The reconstituted formulation may be administered to a mammal, such as a human, in accord with known methods, such as intravenous administration as a bolus or by continuous infusion over a period of time, by intramuscular, intraperitoneal, intracerobrospinal, subcutaneous, intra-articular, intrasynovial, intrathecal, intraventricular, intracerebroventricular, epidural, oral, topical, inhalation routes.

In embodiments, the reconstituted formulation may be administered to the mammal by subcutaneous (i.e. beneath the skin) administration. For such purposes, the reconstituted formulation may be injected using a syringe. However, other devices for administration of the reconstituted formulation are available such as injection devices (e.g. the Inject-ease™ and Genject™ devices); injector pens (such as the GenPen™); needleless devices (e.g. MediJector™ and BioJector™); and subcutaneous patch delivery systems.

The appropriate dosage or therapeutically effective amount of the RAGE fusion protein will depend, for example, on the condition to be treated, the severity and course of the condition, whether the RAGE fusion protein is administered for preventive or therapeutic purposes, previous therapy, the patient's clinical history and response to the RAGE fusion protein, and the discretion of the attending physician. The RAGE fusion protein may be administered to the subject at one time or over a series of treatments. The RAGE fusion protein may be administered as the sole treatment or in conjunction with other drugs or therapies, by way of simultaneous, sequential, or subsequent administrations. In an embodiment, a dosage from about 0.1-20 mg/kg is an initial candidate dosage for administration to the subject, whether, for example, by one or more separate administrations. As described above, other dosage regimens may be useful.

Articles of Manufacture

In another embodiment of the invention, an article of manufacture is provided which contains the formulation of the present invention and may provide instructions for its reconstitution and/or use. The article of manufacture may comprise a container. Suitable containers include, for example, bottles, vials (e.g. dual chamber vials), syringes (such as dual chamber syringes) and test tubes. The container may be formed from a variety of materials such as glass or plastic. The container may hold a lyophilized formulation. In certain embodiments, there may be a label affixed to, or associated with, the container. The label may indicate instructions for reconstitution and/or use. For example, in certain embodiments, the label may indicate that the lyophilized formulation is reconstituted to protein concentrations as described above. The label may further indicate that the formulation is useful or intended for subcutaneous administration.

The container holding the formulation may allow for repeat administrations (e.g., from 2-6, or 2-10, or 2-50 administrations) of the formulation. The article of manufacture may further include other materials desirable from a commercial and user standpoint, including other buffers, diluents, filters, needles, syringes, and package inserts with instructions for use.

### EXAMPLES

Features and advantages of the inventive concept covered by the present invention are further illustrated in the examples which follow.

### Example 1A

### Production of RAGE Fusion Proteins

Two plasmids were constructed to express RAGE-IgG fusion proteins. Both plasmids were constructed by ligating different lengths of a 5′ cDNA sequence from human RAGE with the same 3′ cDNA sequence from human IgG Fc (γ1). These expression sequences (i.e., ligation products) were then inserted in pcDNA3.1 expression vector (Invitrogen, CA). The nucleic acid sequences that encode two wild type RAGE fusion proteins are shown in FIGS. 2 and 3. For TTP-4000 RAGE fusion protein, the nucleic acid sequence from 1 to 753 (highlighted in bold) encodes the RAGE N-terminal protein sequence, whereas the nucleic acid sequence from 754 to 1386 encodes the IgG protein sequence (FIG. 2). For TTP-3000, the nucleic acid sequence from 1 to 408 (highlighted in bold) encodes the RAGE N-terminal protein sequence, whereas the nucleic acid sequence from 409 to 1041 encodes the IgG protein sequence (FIG. 3).

To produce the RAGE fusion proteins, the expression vectors comprising the nucleic acid sequences of either SEQ ID NO: 30 or SEQ ID NO: 31 were stably transfected into CHO cells. Positive transformants were selected for neomycin resistance conferred by the plasmid and cloned. High producing clones as detected by Western Blot analysis of supernatant were expanded and the gene product was purified by affinity chromatography using Protein A columns. Expression may be optimized so that cells may produce recombinant TTP-4000 at levels of about 1.3 grams per liter.

### Example 1B

### Alternate Production of Four Domain RAGE Fusion Proteins

A plasmid was constructed to express RAGE-IgG fusion proteins. The plasmid was constructed by ligating a 5′ cDNA sequence from human RAGE with a 3′ cDNA sequence from human IgG Fc(γ1). PCR was used to amplify the cDNA. Further, on the 5′ end, the PCR primer added an Eco RI restriction enzyme site from cloning and a Kozak consensus translation initiation sequence. On the 3′ end, the PCR primer added a Xho I restriction just past the terminal codon. On the 3′ end, the PCR primer also included two silent base changes that remove a cryptic RNA splice site in the immunoglobulin portion near the terminal codon. The codon encoding for proline (residue 459 based on numbering in the protein sequence in SEQ ID NO: 32) was changed from CCG to CCC, and the codon encoding for glycine (residue 460 based on numbering in the protein sequence in SEQ ID NO: 32) was changed from GGT to GGG. The PCR fragment was digested with Eco RI and Xho I and then inserted into a retrovector plasmid (pCNS-newMCS-WPRE (new ori), available from Gala, Inc.) that had been digested with Mfe I (to form a compatible end with Eco RI) and digested with Xho I. The inserted portion of the cloned plasmid and cloning junctions were sequenced to ensure that no mutations occurred during cloning.

To produce the RAGE-IgG fusion protein, the expression vector comprising the nucleic acid sequence SEQ ID NO: 54 was stably transfected in CHO cells.

The sequence of the RAGE fusion protein TTP-4000 expressed by the transfected cells was confirmed by various characterization studies as either SEQ ID NO: 34 or SEQ ID NO: 56, or both SEQ ID NO: 34 and SEQ ID NO: 56. Thus, the signal sequence encoded by the first 23 amino acids of SEQ ID NO: 32 was cleaved and the N-terminal residue was glutamine (Q) or pyroglutamic acid (pE) or a mixture thereof. Characterization studies also showed glycosylation sites at N2 and N288 (based on numbering of SEQ ID NO: 34 or SEQ ID NO: 56) and showed that the C_{H}3 region of the RAGE fusion protein may have its C-terminal residue cleaved off through a post-translational modification when expressed in this recombinant system.

### Example 1C

### Alternate Production of Three Domain RAGE Fusion Proteins

A plasmid can be constructed to express three domain RAGE-IgG fusion proteins (e.g., one RAGE domain and two IgG domains) such as TTP-3000 in the manner described above for TTP-4000. The plasmid is constructed by ligating a 5′ cDNA sequence from human RAGE encoding amino acids 1-136 of human RAGE with a 3′ cDNA sequence from human IgG Fc(γ1). PCR can be used to amplify the cDNA. On the 5′ end, a PCR primer may add a restriction site (e.g., an Eco RI restriction enzyme site as used for TTP-4000) for cloning and a Kozak consensus translation initiation sequence. On the 3′ end, the PCR primer may also add a restriction site (e.g., a Xho I restriction site) just past the terminal codon. The PCR primers may also include silent base changes as may be needed to remove any cryptic RNA splice sites, such as the cryptic RNA splice sites located at the 3′ end of the immunoglobulin C_{H}2 domain as describes in Example 1B. To remove these cryptic splice sites, the codon encoding for proline 344 of SEQ ID NO: 35 (i.e., residues 1030-1032 based on numbering in the DNA sequence in SEQ ID NO: 31) may be changed from CCG to CCC, and the codon encoding for glycine 345 of SEQ ID NO: 35 (residues 1033-1035 based on numbering in the DNA sequence in SEQ ID NO: 31) may be changed from GGT to GGG. The PCR fragment may then be digested with the appropriate restriction enzymes (e.g., Eco RI and Xho I), and inserted into the retrovector plasmid pCNS-newMCS-WPRE (new ori; available from Gala, Inc.). The vector may be digested with Mfe I to form a compatible end with Eco RI, and also digested with Xho I. The inserted portion of the cloned plasmid and cloning junctions can be sequenced to ensure that no mutations occurred during cloning.

To produce the RAGE-IgG fusion protein, the expression vector comprising the nucleic acid sequence SEQ ID NO: 55 (i.e., comprising the change in DNA sequence to remove cryptic splice sites) can be stably transfected in CHO cells as described in Example 1A and 1B.

The sequence of the RAGE fusion protein TTP-3000 expressed by the transfected cells may be either SEQ ID NO: 36, SEQ ID NO: 37 or SEQ ID NO: 57, or a combination of SEQ ID NO: 36, SEQ ID NO: 37 and/or SEQ ID NO: 57. Thus, the signal sequence encoded by the first 22 and/or 23 amino acids of SEQ ID NO: 35 may be cleaved and the N-terminal residue may be glutamine (Q) or pyroglutamic acid (pE) or a mixture thereof. Glycosylation may occur at sites at N2 and N174 (based on numbering of SEQ ID NO: 37 or SEQ ID NO: 57) and/or other glycosylation sites that may be present. The C_{H}3 region of the RAGE fusion protein may have its C-terminal residue cleaved off through a post-translational modification when expressed in this recombinant system.

### Example 2

### Generation of RAGE Fusion Protein Mutants

Wild type TTP-4000 RAGE plasmid DNA can be used as template for the site-directed mutagenesis and preparation of forward and reverse primers using the following strategies.

To insert a mutation at residue 288, a forward primer containing a mutation site relative the wild type TTP-4000 RAGE plasmid and be 32-34 nucleotides in length can be used. Generally, the primer includes about 10 nucleotides downstream of the mutation site. For example, a forward primer sequence encoding a N288Q mutation is 5′-GCCTCGGGAGGAACAGTAcAgTCCACCTACC (SEQ ID NO: 196), where the bold lowercase letters represent the mutated nucleotides relative to wild type TTP-4000 RAGE plasmid. Using this primer in combination with a reverse primer to amplify the fusion protein DNA by PCR, the amino acid encoded may be changed from wild type asparagine (aAc) to glutamine (cAg). A reverse primer containing an overlapping region with the forward primer at 5′ end of 15-20 nucleotides is used. Thus, a reverse primer sequence may be 5′-TACTGTTCCTCCCGAGGCTTGGTCTTGG (SEQ ID NO: 197) where the underlined letters are an region that overlaps with forward primer of SEQ ID NO: 196.

To insert a mutation R198A at residue 198, the forward primer having the sequence 5′-GCCTCGGCACCGGGCCCTGgcGACCGCCCCTAT (SEQ ID NO: 198) and the reverse primer having the sequence 5′-CAGGGCCCGGTGCCGAGGCAGGCCAGGGGA (SEQ ID NO: 199) is used. Using these primers to amplify the intervening DNA results in changing the wild type arginine (cgG) to alanine (gcG).

The mutants may be generated by PCR using the mutant forward and reverse primer pair with either methylated wild type or mutant plasmid DNA as template using standard amplification conditions based upon the expected primer annealing temperature. PCR products may be transformed into DH5α-Ti chemically competent cells, spread on pre-warmed, LB agar plates with 100 μg/ml ampicillin, incubated at 37° C. overnight. The colonies are then analyzed by colony PCR to confirm the transformants contain an insert having the expected size. The sequence of the mutant DNA sequence was confirmed by DNA sequencing.

Using analogous techniques, each of the SEQ ID NOS: 202 to 220 may be prepared.

### Example 3

### Transfection of HEK and CHO Cells with Clones Expressing Mutant RAGE Fusion Proteins

HEK 293F cells or CHO cells are transfected with the purified plasmid DNA of the TTP-4000 wild type or mutants using Fegene 6 (Roche) in 6-well plate essentially as instructed by the manufacturer. After a 24 hour transfection period, the cells are transferred to 150-mm dishes, and grown in culture medium with 1 mg/mL G418 for selection. After 15-20 days, antibiotic-resistant individual colonies are isolated using 3 mm cloning discs with 0.25% trypsin-EDTA. The collected cells are expanded in 24-well plates and cultured at 37° C., 5% CO₂ humidified incubator using DMEM-10% FBS, 1 mg/mL G418.

The supernatant of each individual clone can be tested for expression of the RAGE fusion protein expression by indirect ELISA using anti-RAGE mAb and anti-human IgG Fc antibodies. Also, the expressed RAGE fusion protein may also be assayed for receptor-ligand binding activity by indirect ELISA using S100b and anti-human IgG Fc-AP antibody. For example, to assay for the RAGE fusion protein, the wells of 96-well ELISA plate are coated with either an anti-RAGE mAb, anti-human IgG Fc polyclonal antibody, or S100b. The plate is then blocked with 1% BSA-TBS, the culture supernatant added, and then alkaline phosphatase-conjugated goat anti-human IgG Fc added to detect binding of the RAGE fusion protein (i.e., the RAGE fusion protein can bind to either the RAGE ligand, the anti-RAGE mAb, or the anti-human IgG antibody that is coated onto the plate). Each incubation is followed by four-time washing of the plates with TBS containing 0.05% Tween 20. The pNPP substrate is added and the absorbance value at 405 nm detected with microplate reader.

The positive clones are further purified by limit dilution and final clones of different RAGE fusion protein mutants can then be used for the protein expression.

### Example 4

### Expression and Purification of TTP-4000

Stable cells of expressing TTP-4000 wild type RAGE fusion protein or a variant (i.e., mutant) thereof are grown at 37° C., 5% CO₂ humidified incubator using DMEM-10% FBS, 0.5 mg/mL G418 in T225 flasks for a week and then transferred to 5 L wave bioreactor or roller bottles. The supernatant is tested for S100b binding activity and the level of expressed protein by indirect ELISA weekly.

After 10-25 days culture, the media is collected and centrifuged at 10,000×g at 4° C. for 30 min. The supernatant is then sequentially filtered with a 0.4 μm filter followed by a 0.2 μm filter. A 1/20 volume of 1M Tris-HCl (pH 8.0) is added to the filtered media (i.e., supernatant) and loaded on protein A column. The column is washed with ˜20 volume of PBS containing 1M of NaCl until OD 280 nm <0.05 on UV detector. The protein is then eluted with 25 mM acetate, pH 3.5 and collected with fraction collector. The eluate is adjusted to pH 6.5 with 1.5M Tris-base solution and concentrated with Amicon ultra-centrifugal filter devices and dialyzed with phosphate buffered saline (PBS) overnight at 4° C.

The purified protein is tested for S100b binding activity by indirect ELISA. Protein concentration was determined both by ELISA and UV absorbance at 280 nm. The sample of purified protein was also be analyzed for purity, molecular weight, and protein concentration by electrophoresis using SDS-PAGE and microfluidic chip-based automated electrophoresis system.

### Example 5

### Pharmacokinetics of RAGE Fusion Proteins in Mice

This experiment examined the pharmacokinetics and in vitro stability of RAGE fusion proteins of the invention in CD-1 mice after IV administration of RAGE fusion proteins at 10 mg/kg. The concentration of RAGE fusion proteins (e.g., TTP4000 and variants thereof as delineated below) in mouse plasma was determined using an enzyme linked immunosorbant assay (ELISA) designed to measure fusion protein by detection of the RAGE and the immunoglobulin portions of the fusion protein. In this assay, the 96 well microtiter plates were coated with 100 μL/well of a RAGE monoclonal antibody (R & D Systems, #MAB1145) at 1 μg/mL in carbonate buffer, pH 9.6. After an overnight incubation at 4° C., the plates were washed with wash buffer (0.05% Tween-20, phosphate buffered saline (PBS)) and treated with a blocking buffer (3% bovine serum albumin (BSA), 0.05% Tween-20, PBS) for about 2 hours at room temperature. The assay plates were then washed with 0.05% Tween-20, PBS wash buffer and 100 μL/well of either: (1) TTP4000 plasma calibration standards (i.e., samples fortified with known concentrations of TTP4000 and then serially diluted); (2) quality controls (i.e., samples fortified with known concentrations of TTP4000 that define the quantitative range of the assay and serve to monitor assay performance); and (3) mouse plasma samples diluted in sample diluent (i.e., 3% BSA, 0.05% Tween-20, PBS) were loaded onto the plates. The plates were then incubated for 1 hour at room temperature on a plate shaker. The plates were then washed with 0.05% Tween-20, PBS and 100 μL/well of the detection antibody (biotinylated mouse anti-human IgG, Fc specific from Jackson Immuno Research, diluted 1:1000) was added followed by a 1 hour incubation at room temperature with shaking. The plates were washed again with 0.05% Tween-20, PBS wash buffer and 100 μL/well of a freshly prepared dilution (1:100,000) of streptavidin/HRP was added and the plates were incubated for 30 minutes with shaking at room temperature. Following the 30 minute incubation, the plates were washed and 100 μL/well of a 3,3′,5′5′-Tetramethlybenzidine (TMB) substrate was added and allowed to incubate at room temperature for about 10 minutes without shaking. The reaction was stopped by the addition of 100 μL/well of 2M H₂SO₄. Absorbance was determined using a Molecular Devices spectrophotometer (Lm1-Lm2: Lm1=450 nm, Lm2=650 nm). Plasma concentrations for the various RAGE fusion proteins were determined using SoftMax Pro software, 4 parameter fit.
RAGE fusion proteins used in this experiment were as follows
Batch 1—SEQ ID NO: 56 from CHO cells (wild type);
Batch 2—SEQ ID NO: 56 from HEK cells (wild type);
Batch 3—SEQ ID NO: 67 from HEK cells (R198A mutation relative to wild type);
Batch 4—SEQ ID NO: 71 from HEK cells (R198A and N2Q mutation relative to wild type);
Batch 5—SEQ ID NO: 72 from HEK cells (R198A and N58Q mutation relative to wild type)

As shown in FIG. 9, RAGE fusion proteins of Batch 3 (R198A) and Batch 4 (R198A and N2Q) remained at higher plasma concentrations over time as compared to other tested RAGE fusion proteins (FIG. 9A and FIG. 9B). This effect was observed over time periods from 0-336 hours (FIG. 9A) and 0-48 hours (FIG. 9B).

FIG. 10 shows mean pharmacokinetic parameters for the tested RAGE fusion proteins of Batches 1-5 following intravenous administration to mice at 10 mg/kg. Overall drug exposure as assessed by area under the curve (AUC) was greater in the RAGE fusion proteins of Batch 4 (R198A and N2Q) and Batch 5 (R198A and N58Q) than for Batches 1 and 2 (wild type) and Batch 3 (R198A). The total body clearance (Clp) for the RAGE fusion proteins of Batch 4 (R198A and N2Q) and Batch 5 (R198A and N58Q) were lower than for Batches 1 and 2 (wild type) and Batch 3 (R198A). The steady state volume of distribution (Vss) for the RAGE fusion proteins of Batch 4 (R198A and N2Q) and Batch 5 (R198A and N58Q) were lower than for Batches 1 and 2 (wild type) and Batch 3 (R198A).

FIG. 11 shows in vitro stability data for the tested RAGE fusion proteins of Batches 1 to 5 described above. FIG. 11A provides this data in tabular form, while FIG. 11B provides this data in graphic form. Fresh mouse plasma (drug concentration at 1.0 μg/mL) was incubated at 37° C., and samples were obtained from 0-48 hours.

### Example 6

### Measurement of Ligand Binding of Mutant RAGE Fusion Proteins

The supernatant from individual clones was used to measure expression of mutant TTP-4000 proteins and the ability of the expressed proteins to bind to the RAGE ligand S100b.

The binding assay was an indirect ELISA using S100b as the RAGE ligand, and an anti-human IgG Fc antibody labeled with alkaline phosphatase (AP) to detect RAGE binding to the S100b. The wells of 96-well ELISA plate were coated with 100 ul/well, 5 ug/ml of S100b overnight at 4° C. using 100 nM of bicarbonate/carbonate coating buffer (pH 9.6). The plate was then blocked with 300 ul/well of 1% BSA-TBST at room temperature for 2 hrs. Next, 100 ul/well of culture supernatant or purified mutant RAGE fusion protein was added, and then 100 ul/well of alkaline phosphatase-conjugated goat anti-human IgG Fc, 1:2500 dilution with 1% BSA-TBST was added. Each incubation was followed by four separate washes with TBS containing 0.05% Tween 20 (TBST). The alkaline phosphatase substrate, para-nitrophenyl phosphate (pNPP), was added and the absorbance value at 405 nm was detected with microplate reader and normalized to weight percent (wt %). Results are shown in FIG. 12.

All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

1-29. (canceled)

30. A fusion protein comprising a Receptor for Advanced Glycation Endproducts (RAGE) polypeptide linked to an immunoglobulin polypeptide, wherein the RAGE polypeptide comprises a fragment of a mammalian RAGE having a ligand binding domain, and wherein the RAGE fusion protein comprises at least one mutation relative to the wild-type sequence in at least one of the RAGE polypeptide or the immunoglobulin polypeptide, wherein the mutation removes and/or alters at least one of a glycosylation site or an enzyme cleavage site.

31. The fusion protein of claim 30, wherein the RAGE polypeptide is a human RAGE polypeptide.

32. The fusion protein of claim 30, wherein the mutation changes the sequence in the wild-type RAGE polypeptide present at a glycosylation site.

33. The fusion protein of claim 30, wherein the glycosylation site has the amino acid sequence NXS or NXT, where X is any amino acid.

34. The fusion protein of claim 30, wherein the mutation changes the sequence in the wild-type RAGE polypeptide from at least one of NIT to QIT, or NGS to QGS, or NGS to NSS, or NST to QST to remove at least one glycosylation site.

35. The fusion protein of claim 30, wherein the glycosylation site is within the ligand binding site or the ligand binding domain of the RAGE polypeptide.

36. The fusion protein of claim 30, wherein the enzyme cleavage site is a furin cleavage site, such that the mutation changes the sequence in the wild-type RAGE polypeptide present at a recognition site for furin cleavage of the RAGE polypeptide.

37. The fusion protein of claim 30, wherein the mutation changes the amino acid sequence in the wild-type RAGE polypeptide from one of: (i) PRHRALR (SEQ ID NO:226) to PHRAALR (SEQ ID NO:227); (ii) PRHRALR to PRHKALR (SEQ ID NO:228); (iii) PRHRALR to PRHRALA (SEQ ID NO:229); (iv) PRHRALR to PRHRALK (SEQ ID NO:230); (v) PRHRALR to PRHRALH (SEQ ID NO:231); or (vi) PRHRALR to PRHRALT (SEQ ID NO:232) to remove a furin cleavage site.

38. The fusion protein of claim 30, wherein the RAGE polypeptide does not include an N-terminal leader peptide consisting of amino acids 1-23 of the wild-type RAGE polypeptide.

39. The fusion protein of claim 30, wherein at least one mutation comprises at least one of following: (i) N2Q; (ii) N58Q; (iii) G59S; (iv) N2Q and N58Q; or (v) N2Q and G59S of a human RAGE polypeptide that does not include an N-terminal leader peptide consisting of amino acids 1-23 of the wild-type RAGE polypeptide.

40. The fusion protein of claim 39, wherein the human RAGE polypeptide that does not include an N-terminal leader peptide consisting of amino acids 1-23 of the wild-type RAGE polypeptide comprises the sequence as set forth in at least one of: (i) SEQ ID NO: 8, SEQ ID NO: 16, or SEQ ID NO: 20; or (ii) SEQ ID NO: 8, SEQ ID NO: 16, or SEQ ID NO: 20 having the N-terminal glutamine cyclized to form pyroglutamic acid.

41. The fusion protein of claim 30, wherein the mutation to remove the furin cleavage site comprises at least one of R195A, R195K, R195T, R195H, R198A, R198K, R198H, R198T of a human RAGE polypeptide that does not include an N-terminal leader peptide consisting of amino acids 1-23 of the wild-type RAGE polypeptide.

42. The fusion protein of claim 41, wherein the human RAGE polypeptide that does not include an N-terminal leader peptide consisting of amino acids 1-23 of the wild-type RAGE polypeptide comprises the sequence as set forth in SEQ ID NO: 20 or SEQ ID NO: 20 having the N-terminal glutamine cyclized to form pyroglutamic acid.

43. The fusion protein of claim 30, wherein the immunoglobulin polypeptide comprises a fragment of a C_{H}2 domain which does not include at least a portion of the hinge region.

44. The fusion protein of claim 43, wherein the hinge region that is not included in the fragment of the C_{H}2 domain has the sequence as set forth in SEQ ID NO: 223 or SEQ ID NO: 224.

45. The fusion protein of claim 43, wherein the C_{H}2 domain is linked via its C-terminus to the N-terminus of a C_{H}3 domain of an immunoglobulin polypeptide.

46. The fusion protein of claim 30, wherein the immunoglobulin comprises a human IgG.

47. The fusion protein of claim 30, wherein the C_{H}2 domain of the immunoglobulin comprises SEQ ID NO: 38, or SEQ ID NO: 38 without the C-terminal lysine.

48. The fusion protein of claim 30, wherein the fusion protein is in the form of a monomer, a dimer, a trimer, a tetramer, or a mixture thereof.

49. A fusion protein comprising a RAGE polypeptide comprising an amino acid sequence as set forth in any one of SEQ ID NOs: 58-82, SEQ ID NOs: 92-190, or SEQ ID NOs: 200 or 201.

50. A fusion protein comprising a RAGE polypeptide wherein the RAGE polypeptide sequence is as set forth in any one of SEQ ID NOs: 58-82, SEQ ID NOs: 92-190, or SEQ ID NOs: 200 or 201.

51. A composition comprising a fusion protein comprising a Receptor for Advanced Glycation Endproducts (RAGE) polypeptide linked to an immunoglobulin polypeptide, wherein the RAGE polypeptide comprises a fragment of a mammalian RAGE having a ligand binding domain, and wherein the RAGE fusion protein comprises at least one mutation relative to the wild-type sequence in at least one of the RAGE polypeptide or the immunoglobulin polypeptide, wherein the mutation removes and/or alters at least one of a glycosylation site or an enzyme cleavage site and a pharmaceutically acceptable carrier.

52. An isolated nucleic acid encoding a fusion protein comprising a Receptor for Advanced Glycation Endproducts (RAGE) polypeptide linked to an immunoglobulin polypeptide, wherein the RAGE polypeptide comprises a fragment of a mammalian RAGE having a ligand binding domain, and wherein the RAGE fusion protein comprises at least one mutation relative to the wild-type sequence in at least one of the RAGE polypeptide or the immunoglobulin polypeptide, wherein the mutation removes and/or alters at least one of a glycosylation site or an enzyme cleavage site.

53. An expression vector comprising a nucleic acid encoding a fusion protein comprising a Receptor for Advanced Glycation Endproducts (RAGE) polypeptide linked to an immunoglobulin polypeptide, wherein the RAGE polypeptide comprises a fragment of a mammalian RAGE having a ligand binding domain, and wherein the RAGE fusion protein comprises at least one mutation relative to the wild-type sequence in at least one of the RAGE polypeptide or the immunoglobulin polypeptide, wherein the mutation removes and/or alters at least one of a glycosylation site or an enzyme cleavage site.

54. A host cell transfected with an expression vector comprising a nucleic acid encoding a fusion protein comprising a Receptor for Advanced Glycation Endproducts (RAGE) polypeptide linked to an immunoglobulin polypeptide, wherein the RAGE polypeptide comprises a fragment of a mammalian RAGE having a ligand binding domain, and wherein the RAGE fusion protein comprises at least one mutation relative to the wild-type sequence in at least one of the RAGE polypeptide or the immunoglobulin polypeptide, wherein the mutation removes and/or alters at least one of a glycosylation site or an enzyme cleavage site.

55. A method for treating a RAGE-mediated disorder in a subject comprising administering to a subject a composition comprising a fusion protein comprising a Receptor for Advanced Glycation Endproducts (RAGE) polypeptide linked to an immunoglobulin polypeptide, wherein the RAGE polypeptide comprises a fragment of a mammalian RAGE having a ligand binding domain, and wherein the RAGE fusion protein comprises at least one mutation relative to the wild-type sequence in at least one of the RAGE polypeptide or the immunoglobulin polypeptide, wherein the mutation removes and/or alters at least one of a glycosylation site or an enzyme cleavage site.

56. The method of claim 55, wherein the RAGE-mediated disorder comprises a symptom of diabetes or diabetic late complications.

57. The method of claim 56, wherein the symptom of diabetes or diabetic late complications comprises at least one of diabetic nephropathy, diabetic retinopathy, a diabetic foot ulcer, a cardiovascular complication, or diabetic neuropathy.

58. The method of claim 55, wherein the RAGE-mediated disorder comprises at least one of amyloidosis, Alzheimer's disease, cancer, kidney failure, inflammation associated with autoimmunity, inflammatory bowel disease, rheumatoid arthritis, psoriasis, multiple sclerosis, hypoxia, stroke, heart attack, hemorrhagic shock, sepsis, organ transplantation, or impaired wound healing.
